(19) United States
(12) Patent Application Publication
(10) Pub. No.: US 2010/0210710 A1
(43) Pub. Date: Aug. 19, 2010

(54) THERAPEUTIC siRNA MOLECULES FOR REDUCING VEGFR1 EXPRESSION IN VITRO AND IN VIVO

(75) Inventors: Frank Y. Xie, Germantown, MD (US); Yijia Liu, Gaithersburg, MD (US); Xiaodong Yang, Palo Alto, CA (US)

Correspondence Address: ROPES & GRAY LLP, PATENT DOCKETING 39/361, 1211 AVENUE OF THE AMERICAS, NEW YORK, NY, 10036-8704, US

(73) Assignee: INTRADIGM CORPORATION, Palo Alto, CA (US)

(21) Appl. No.: 12/682,615

(22) PCT Filed: Oct. 10, 2008
(86) PCT No.: PCT/US08/11645

§ 371 Date: Apr. 12, 2010

Related U.S. Application Data
(60) Provisional application No. 60/998,631, filed on Oct. 12, 2007.

(51) Int. Cl. A61K 31/713; C07H 21/02; A61K 31/7105; C12N 5/071; A61P 35/00

(52) U.S. Cl. 514/44 A; 536/24.5; 435/325

(57) ABSTRACT

The invention relates to nucleic acid molecule compositions for use in modulating the expression and activity of VEGF pathway genes and decreasing unwanted neovascularization, including tumor angiogenesis, by RNA interference and methods and compositions comprising the nucleic acid molecules.

### CROSS-REFERENCE TO RELATED APPLICATIONS

This application claims priority under 35 U.S.C. §119(e) from U.S. provisional application 60/998,631, filed Oct. 12, 2007. The contents of 60/998,631 are hereby incorporated by reference in their entirety.

### FIELD OF THE INVENTION

The present invention is in the field of molecular biology and medicine and relates to RNA interference (RNAi)-inducing compositions and methods of using them to modulate the expression of VEGF pathway genes, such as VEGFR1, in vitro and in vivo to treat conditions and diseases with unwanted neovascularization.

### BACKGROUND OF THE INVENTION

The invention provides compositions and methods for treatments of diseases with unwanted neovascularization (NV), often an abnormal or excessive proliferation and growth of blood vessels. The development of NV itself often times has adverse consequences or it can be an early pathological step in disease. Despite introduction of new therapeutic antagonists of angiogenesis including antagonists of the Vascular Endothelial Growth Factor (VEGF) pathway, treatment options for controlling NV are inadequate and a large and growing unmet clinical need remains for effective treatments of NV, either to inhibit disease progression or to reverse unwanted angiogenesis.

The VEGF pathway includes the angiogenic factor VEGF and its tyrosine kinase receptors VEGFR1 (Flt-1) and VEGFR2 (KDR). Soluble VEGFR1 (sVEGFR1; sFlt-1) is a splice variant of membrane-bound full-length VEGFR1 that lacks the transmembrane and cytoplasmic domains. sVEGFR1 produces an anti-angiogenic effect by sequestering VEGF and forming inactive heterodimers with full-length VEGFR2 (Kendall et al. Biochem Biophys Res Commun. 1996; 226: 324-328, incorporated herein by reference in its entirety). Exogenously expressing sVEGFR1 protein has been shown to have anti-angiogenic effects in cell lines and tumor xenograft models (Mahendra et al. Cancer Gene Therapy 2005; 12:26-34; Kommareddy et al. Cancer Gene Therapy 2007; 14:488-498), incorporated herein by reference in their entirety.

RNA interference (RNAi) is a post-transcriptional process where a double stranded RNA inhibits gene expression in a sequence specific fashion. The RNAi process occurs in at least two steps: During one step, a long dsRNA is cleaved by an endogenous ribonuclease into shorter, 21- or 23-nucleotide-long dsRNAs by a RNase III-like activity involving the enzyme Dicer. In a second step, the smaller dsRNA mediates the degradation of an mRNA molecule with a matching sequence in a multi-protein RNA-induced silencing complex (RISC) and as a result selectively down regulates expression of that gene. This RNAi effect can be achieved by introduction of either longer double-stranded RNA (dsRNA) or shorter small interfering RNA (siRNA) to the target sequence within cells. RNAi can also be achieved by introducing a plasmid that generate dsRNA complementary to target gene.

Improved methods for delivering RNAi-inducing molecules in vivo are of great importance. It is also apparent that tissue targeted delivery of nucleic acid molecules inducing RNAi is of great importance. It is also apparent that methods for delivering nucleic acid molecules inducing RNAi selective for VEGF pathway genes will be of great benefit for the treatment of NV diseases. These needs are addressed by the compositions and methods of the invention.

### SUMMARY OF THE INVENTION

VEGF-mediated antiogenesis and NV can be reduced by antagonists targeted at VEGF, VEGFR1, and/or VEGFR2. VEGFR1 is produced in a secreted “soluble” form as a splice variant of the full-length “membrane-bound” form. Soluble VEGFR1 acts as a VEGF pathway antagonist by sequestering VEGF so that it can no longer free to bind to full-length VEGFR1 and by forming inactive heterodimers with full-length VEGFR2 (Kendall et al. Biochem Biophys Res Commun. 1996; 226: 324-328, incorporated herein by reference in its entirety).

It is therefore an object of present invention to provide nucleic acid molecules for use in inducing RNAi of VEGFR1 to modulate the angiogenesis process and/or to reverse the disease process by down regulating gene expression involved in NV pathogenesis. The inventors unexpectedly found RNAi-inducing nucleic acid molecules that target and reduce the expression of full-length VEGFR1 and surprisingly also increase the expression of soluble VEGFR1. Thus, these nucleic acid molecules provide the advantageous property of simultaneously reducing the pro-angiogenic activity of full-length VEGFR1, VEGF, and VEGFR2.

In one embodiment of the invention, the nucleic acid molecules reduce the expression of full-length VEGFR1 mRNA or protein levels while not affecting the expression of soluble VEGFR1 mRNA or protein levels. In another embodiment of the invention, the nucleic acid molecules increase the expression of total VEGFR1 mRNA or protein levels while increasing the expression of soluble VEGFR1 mRNA or protein levels. In another embodiment of the invention, the nucleic acid molecules decrease the expression of total VEGFR1 mRNA or protein levels while increasing the expression of soluble VEGFR1 mRNA or protein levels. In a preferred embodiment, the nucleic acid molecules reduce the expression of full-length membrane-bound VEGFR1 mRNA or protein levels while increasing the expression of soluble VEGFR1 mRNA or protein levels.

One aspect of the invention is to provide compositions and methods for inhibiting expression of VEGFR1 in combination with one or more other VEGF pathway genes in a mammal. It is a further aspect of the invention to provide compositions and methods for treating NV disease by inhibiting expression of VEGFR1 alone, in combination with inhibiting expression of one or more other VEGF pathway genes, or in combination with other agents including antagonists of the VEGF pathway.

The invention provides compositions and methods for down regulating VEGFR1 gene expression, comprising administering to a tissue of a mammal a composition comprising a nucleic acid molecule wherein the nucleic acid molecule specifically reduces or inhibits expression of VEGFR1. This down regulation of an endogenous gene may be used for treating a disease that is caused or exacerbated by activity of the VEGF pathway. The disease may be in a human.

Also provided are methods for treating a disease in a mammal associated with undesirable expression of a VEGF pathway gene, comprising administering a nucleic acid composition comprising a dsRNA oligonucleotide, as the active pharmaceutical ingredient (API), associated with a formulation, wherein the formulation can be comprised of a polymer, where the nucleic acid composition is capable of reducing expression of the VEGF pathway genes and inhibiting NV in the disease. The disease may be cancer or a precancerous growth and the tissue may be, for example, a kidney tissue, breast tissue, colon tissue, a prostate tissue, a lung tissue, or an ovarian tissue. One aspect of the present invention provides compositions and methods for treatment of cancer or pre-cancerous growths or conditions. In another aspect of the present invention, nucleic acid agents inducing RNAi are used in concert with other therapeutic agents, such as but not limited to small molecules and monoclonal antibodies (mAb), in the same therapeutic regimen.

Any of the methods of the invention may be carried out using any of the APIs of the invention or any of the compositions provided herein for modulating the expression of VEGFR1, or VEGFR1 in combination with one or more VEGF pathway genes, by inhibiting, reducing, or increasing the expression. In one embodiment, the API or composition for inhibiting or reducing expression of one or more VEGF pathway genes comprises at least one siRNA that inhibits or reduces expression of VEGF. In another embodiment the API or composition for inhibiting or reducing expression of one or more VEGF pathway genes comprises at least one siRNA that inhibits or reduces expression of VEGFR1. In yet another embodiment the API or composition for inhibiting or reducing expression of one or more VEGF pathway genes comprises at least one siRNA that inhibits or reduces expression of VEGFR2. In a further embodiment the API or composition for inhibiting or reducing expression of one or more VEGF pathway genes comprises at least one siRNA that inhibits or reduces expression of VEGF and at least one siRNA that inhibits or reduces expression of VEGFR1. In another embodiment the API or composition for inhibiting or reducing expression of one or more VEGF pathway genes comprises at least one siRNA that inhibits or reduces expression of VEGF and at least one siRNA that inhibits or reduces expression of VEGFR1 and at least one siRNA that inhibits or reduces expression of VEGFR2. In another embodiment the API or composition for inhibiting or reducing expression of one or more VEGF pathway genes comprises at least one siRNA that inhibits or reduces expression of VEGFR1 and at least one siRNA that inhibits expression of VEGFR2. In one embodiment the API or composition for inhibiting or reducing expression of one or more VEGF pathway genes comprises at least one siRNA that inhibits or reduces expression of VEGF, at least one siRNA that inhibits or reduces expression of VEGFR1 and at least one siRNA that inhibits or reduces expression of VEGFR2. In all of the above API or composition for inhibiting or reducing expression of one or more VEGF pathway genes the siRNA that inhibits or reduces expression of VEGF, VEGFR1 or VEGFR2 may be any of the siRNA listed herein.

In one embodiment the API or composition for inhibiting or reducing expression of one or more VEGF pathway genes comprises at least one siRNA selected from any of the siRNAs listed herein. In another embodiment the API or composition for inhibiting or reducing expression of one or more VEGF pathway genes comprises at least two siRNAs selected from any of the siRNAs listed herein. In yet another embodiment the API or composition for inhibiting or reducing expression of one or more VEGF pathway genes comprises at least three siRNAs selected from any of the siRNAs listed herein.

The composition may further comprise a polymeric carrier. The polymeric carrier may comprise a cationic polymer that binds to the RNA molecule and forms nanoparticles. The cationic polymer may be an amino acid copolymer, containing, for example, histidine and lysine residues. The polymer may comprise a branched polymer. The composition may comprise a targeted synthetic vector. The synthetic vector may comprise a cationic polymer as a nucleic acid carrier, a hydrophilic polymer as a steric protective material, and a targeting ligand as a target cell selective agent. The cationic polymer may comprise a polyethyleneimine or a polyhistidine-lysine copolymer or a polylysine modified chemically or other effective polycationic carriers that can be used as the nucleic acid carrier module. The hydrophilic polymer may comprise a polyethylene glycol or a polyacetal or a polyoxazoline and the targeting ligand may comprise a peptide comprising an RGD sequence or a sugar or a sugar analogue or an mAb or a fragment of an mAb, or any other effective targeting moieties.

The compositions and methods of the invention include RNAi-inducing nucleic acid molecules, including dsRNA oligonucleotides, with a sequence that is identical, substantially identical, homologous or substantially homologous to a portion of the VEGFR1 gene. Said gene can be the wildtype gene or a mutated gene. In the case of the mutated gene at least one mutation in the mutated gene may be in a coding or regulatory region of the gene. In any of these methods, the RNAi-inducing nucleic acid molecule that targets VEGFR1 may be used in combination with RNAi-inducing nucleic acid molecule(s) that target genes selected from the group consisting of growth factor genes, protein serine/threonine kinase genes, protein tyrosine kinase genes, protein serine/threonine phosphatase genes, protein tyrosine phosphatase genes, receptor genes, and transcription factor genes. These additional genes may include one or more genes from the group consisting of VEGF, VEGFR2, VEGFR3, VEGF121, VEGF165, VEGF189, VEGF206, RAF-a, RAF-c, AKT, Ras, and NFKb. The additional genes may include one or more genes from other biochemical pathways associated with NV including HIF, EGF, EGFR, bFGF, bFGFR, PDGF, and PDGFR. The additional genes may include one or more genes from other biochemical pathways operative in concert with NV including Her-2, c-Met, c-Myc, and HGF.

The present invention also provides compositions and methods comprising nucleic acid agents that induce RNAi for inhibiting multiple genes, including cocktails of siRNA (siRNA-OC). The compositions and methods of the invention may inhibit multiple genes substantially contemporaneously or they may inhibit multiple genes sequentially. In a preferred embodiment, siRNA-OC agents inhibit three VEGF pathway genes: VEGF, VEGFR1, and VEGFR2. In another preferred embodiment, siRNA-OC are administered substantially contemporaneously.

The present invention provides nucleic acid molecules with gene inhibition selectivity derived from substantial complementarity to a sequence in the VEGFR1 mRNA. It also provides methods for treatment of human diseases, especially NV related diseases, which can be treated with inhibitors of multiple endogenous genes. It also provides methods for treatment of human diseases by combinations of therapeutic agents administered substantially contemporaneously in some cases and sequentially in other cases.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

Throughout this application, various patents, publications and references are referred to. Disclosures of these patents, publications and references are hereby incorporated by reference into this application in their entireties, as if they were referred to individually.

### BRIEF DESCRIPTION OF DRAWINGS

FIG. 1A is a bar graph depicting knockdown of soluble hVEGFR1 protein in HUVEC cells transfected with siRNA targeting mRNAs coding for both soluble and full-length hVEGFR1.

In HUVEC cells, siRNAs (1-19 in Table 4, correspond to hVEGFR1-25-1 to hVEGFR1-25-19 siRNA) targeting mRNAs coding for both soluble and full-length membrane-bound hVEGFR1 significantly reduced the levels of soluble hVEGFR1 protein in cell culture supernatant. HUVEC cells were transfected with 20 nM of siRNA and assayed at 48 hours post transfection for the concentration of hVEGFR1 protein in the culture medium using a commercial hVEGFR1 ELISA kit (R&D). 1-19: hVEGFR1-25-1 to hVEGFR1-25-19 siRNA in Table 4; Mock: mock transfection; Ctrol: negative control siRNA. Data were presented as mean+/−standard deviation.

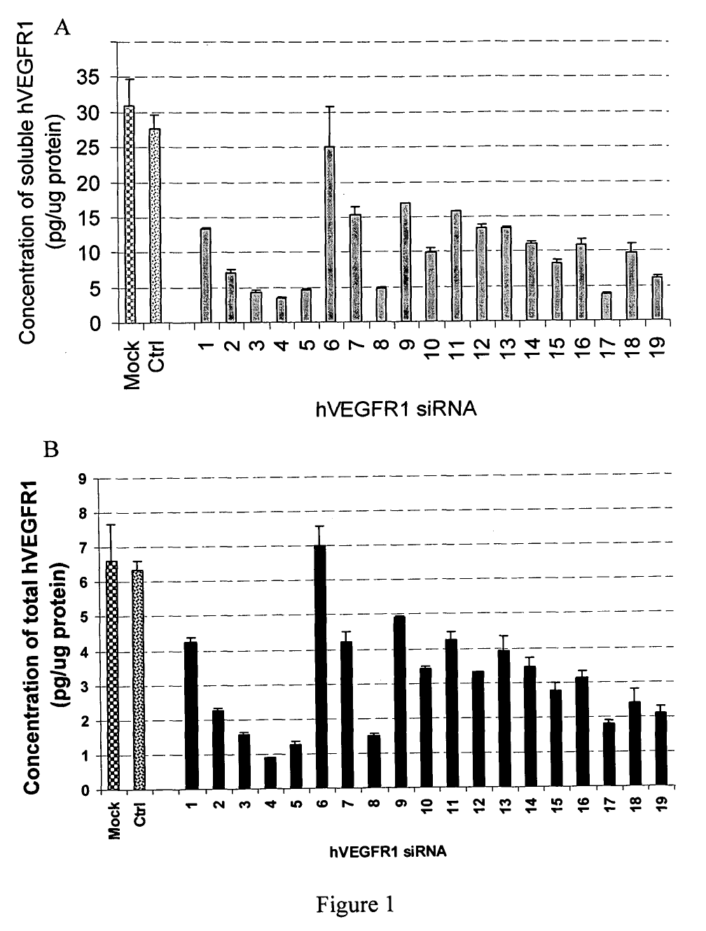

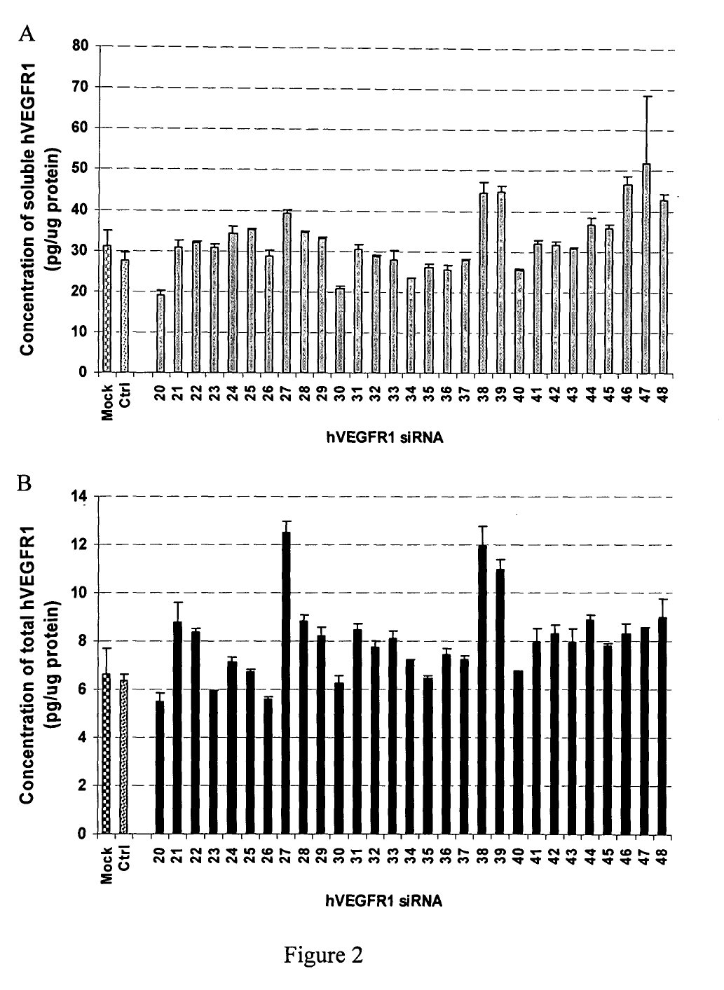

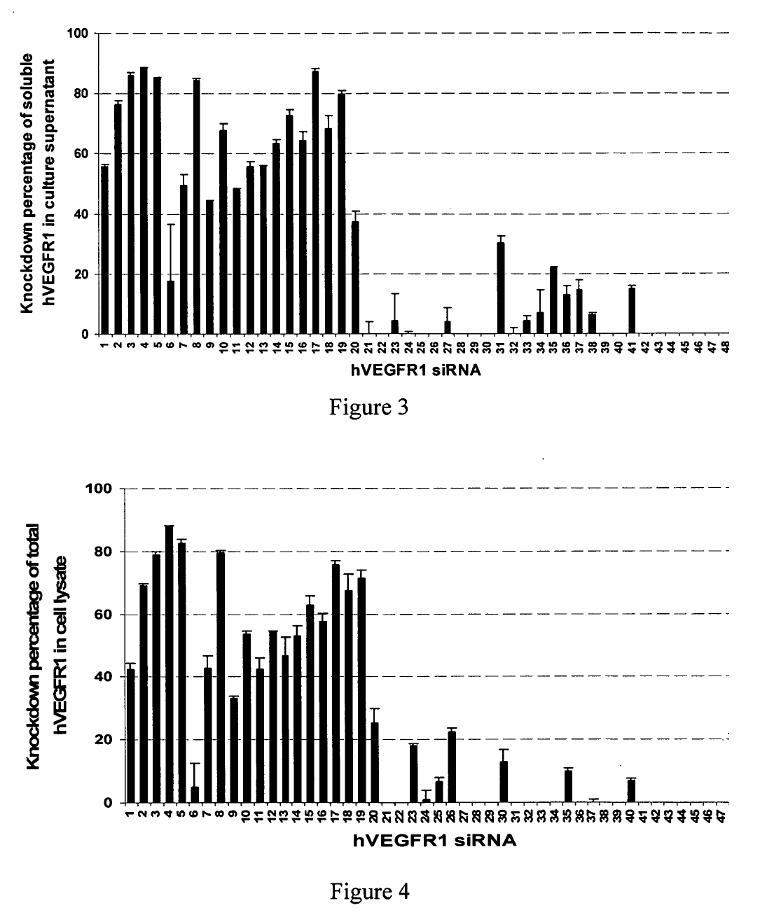

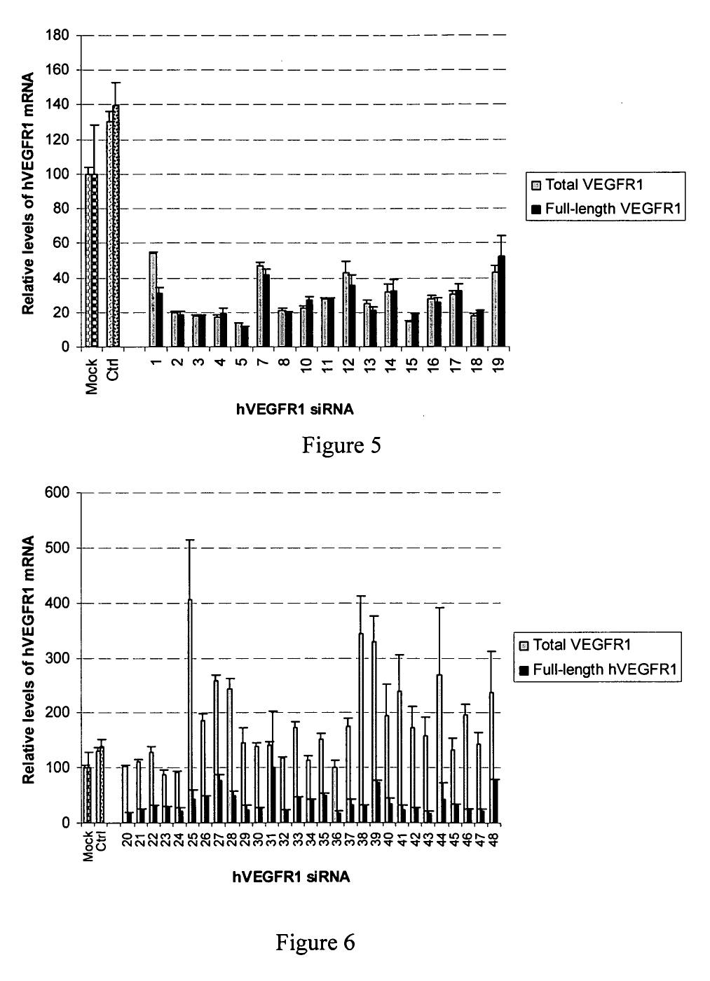

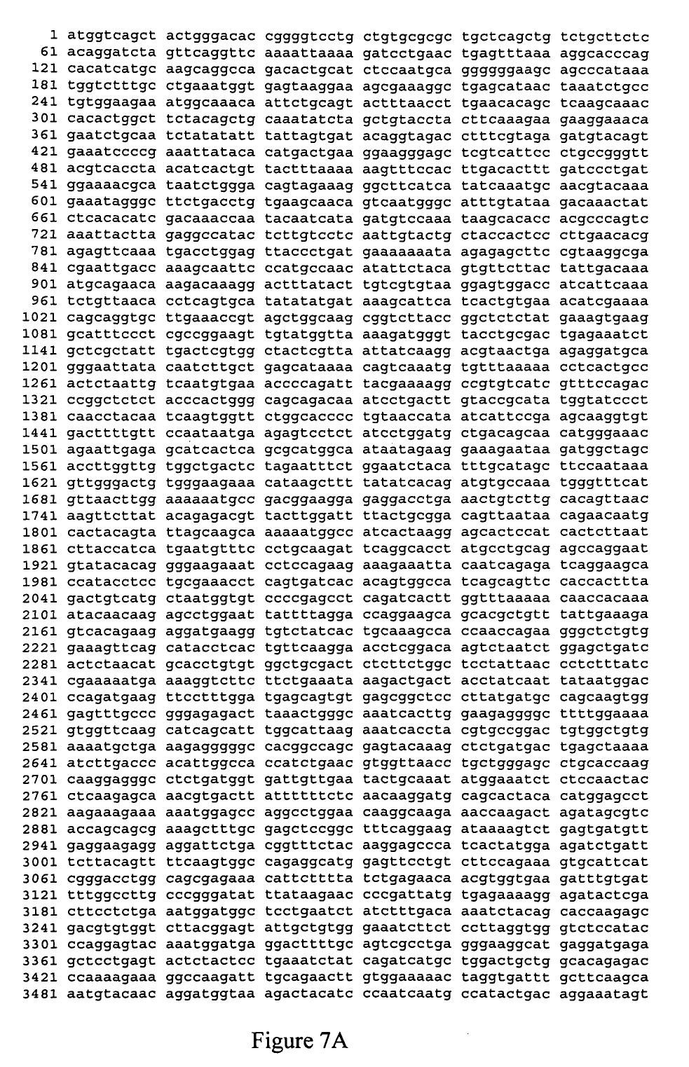

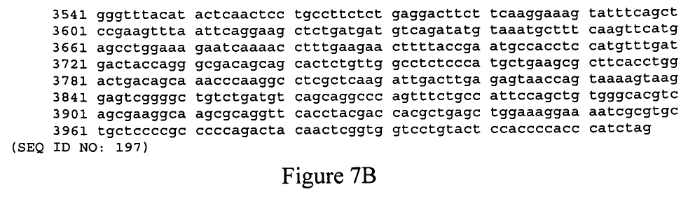

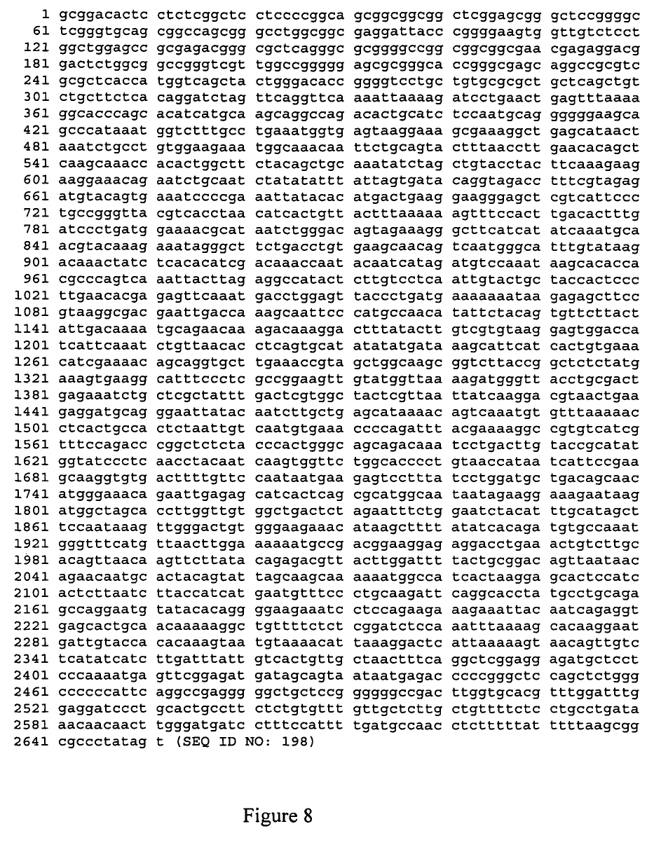

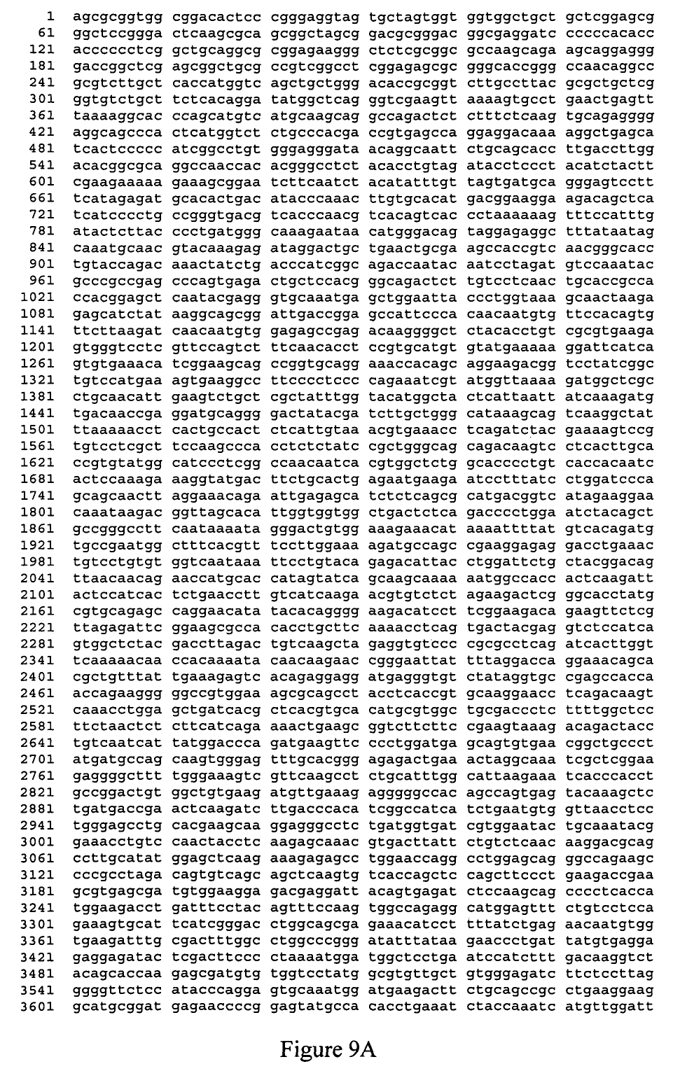

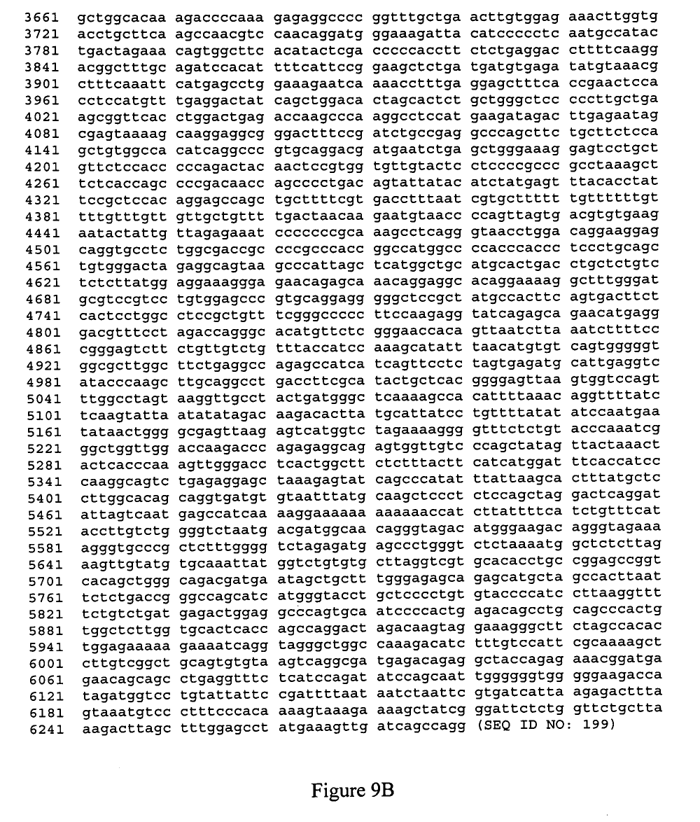

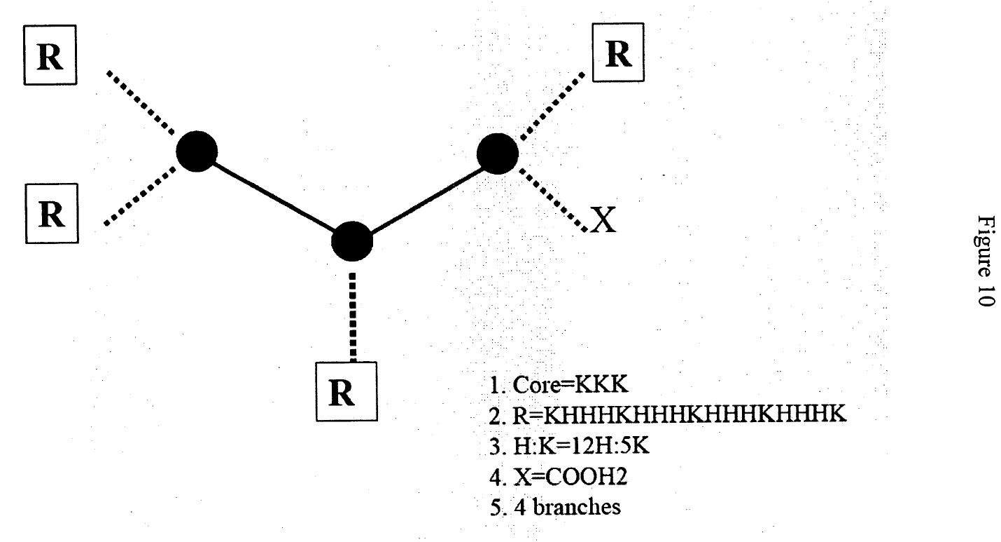

FIG. 1B is a bar graph depicting knockdown of total hVEGFR1 protein in HUVEC cells transfected with siRNA targeting mRNAs coding for both soluble and full-length hVEGFR1.

In HUVEC cells, siRNAs (1-19 in Table 4, hVEGFR1-25-1 to hVEGFR1-25-19 siRNA) targeting mRNAs coding for both soluble and full-length membrane-bound hVEGFR1 significantly reduced the levels of total hVEGFR1 protein in HUVEC cell lysates. HUVEC cells were transfected with 20 nM of siRNA and assayed at 48 hours post transfection for the concentration of hVEGFR1 protein in the cell lysate using a commercial hVEGFR1 ELISA kit (R&D). 1-19: hVEGFR1-25-1 to hVEGFR1-25-19 siRNA in Table 4; Mock: mock transfection; Ctrol: negative control siRNA. Data were presented as mean+/−standard deviation.

FIG. 2A is a bar graph depicting no inhibitory effect on soluble hVEGFR1 protein level by treating HUVEC cells with siRNA specific for full-length hVEGFR1 mRNA.

In HUVEC cells, full-length membrane-bound hVEGFR1 specific siRNAs (20-48 in Table 5, correspond to hVEGFR1-25-20 to hVEGFR1-25-48 siRNA) have no inhibitory effect on the level of soluble hVEGFR1 in cell culture supernatant. HUVEC cells were transfected with 20 nM of siRNA and assayed at 48 hours post transfection for the level of hVEGFR1 protein in the culture medium using a commercial hVEGFR1 ELISA kit (R&D). 20-48: hVEGFR1-25-20 to hVEGFR1-25-48 siRNA in Table 5; Mock: mock transfection; Ctrl: negative control siRNA. Data were presented as mean+/−standard deviation.

FIG. 2B is a bar graph depicting no inhibitory effect on total hVEGFR1 protein level by treating HUVEC cells with siRNA specific for full-length hVEGFR1 mRNA.

In HUVEC cells, full-length membrane-bound hVEGFR1 specific siRNAs (20-48 in Table 5, hVEGFR1-25-20 to hVEGFR1-25-48 siRNA) have no inhibitory effect on the level of total hVEGFR in cell lysate. Because full-length membrane bound hVEGFR1 specific siRNAs knock down mRNA coding for full-length hVEGFR1 (see FIG. 6), they may stimulate the production of soluble hVEGFR1 present in cell lysate. HUVEC cells were transfected with 20 nM of siRNA and assayed at 48 hours post transfection for the level of hVEGFR1 protein in cell lysate using a commercial hVEGFR1 ELISA kit (R&D). 20-48: hVEGFR1-25-20 to hVEGFR1-25-48 siRNA in Table 5; Mock: mock transfection; Ctrl: negative control siRNA. Data were presented as mean+/−standard deviation.

FIG. 3 is a bar graph comparing the effect of siRNAs targeting both soluble and full-length membrane-bound forms of hVEGFR1 (1-19 in Table 4, hVEGFR1-25-1 to hVEGFR1-25-19 siRNA) to the effect of siRNAs targeting membrane form of hVEGFR1 only (20-48, hVEGFR1-25-20 to hVEGFR1-25-48 siRNA in Table 5), on soluble hVEGFR1 secretion in HUVEC cell supernatant at 48 hours post-transfection. The effects are represented by % knockdown of soluble hVEGFR1 levels (as compared to mock transfection).

FIG. 4 is a bar graph comparing the effect of siRNAs targeting both soluble and full-length membrane-bound forms of hVEGFR1 (1-19 in Table 4, hVEGFR1-25-1 to hVEGFR1-25-19 siRNA) to the effect of siRNAs targeting membrane form of hVEGFR1 only (20-48, hVEGFR1-25-20 to hVEGFR1-25-48 siRNA in Table 5), on hVEGFR1 expression as measured in HUVEC cell lysate at 48 hours post-transfection. The effects are represented by % knockdown of total hVEGFR1 levels (as compared to mock transfection).

FIG. 5 is a bar graph depicting knockdown of hVEGFR1 mRNAs in HUVEC cells transfected with siRNAs targeting mRNAs coding for both soluble and full-length membrane-bound hVEGFR1.

In HUVEC cells, siRNA (1-19 in Table 4, hVEGFR1-25-1 to hVEGFR1-25-19 siRNA) targeting mRNAs coding for both soluble and full-length membrane-bound hVEGFR1 significantly reduced the levels of full-length hVEGFR1 mRNA (black bars) and total hVEGFR1 mRNA (gray bars). HUVEC cells were transfected with 10 nM of siRNA and assayed at 48 hours post transfection for the levels of hVEGFR1 mRNAs, using a quantitative RT-PCR assay with a primer set specific for full-length hVEGFR1 mRNA (black bars) or a primer set for both the soluble and full-length hVEGFR1 mRNA (gray bars). 1-19: hVEGFR1-25-1 to hVEGFR1-25-19 siRNA in Table 4; Mock: mock transfection; Ctrl: negative control siRNA. Data were presented as mean+/−standard deviation.

FIG. 6 is a bar graph depicting knockdown of hVEGFR1 mRNAs in HUVEC cells transfected with full-length specific hVEGFR1 siRNAs.

In HUVEC cells, full-length membrane-bound hVEGFR1 specific siRNAs (20-48 in Table 5, hVEGFR1-25-20 to hVEGFR1-25-48 siRNA) significantly reduce only the full-length hVEGFR1 mRNA (black bars), and had no inhibitory effect on the level of total hVEGFR1 mRNAs (gray bars). Therefore, full-length membrane-bound hVEGFR1 specific siRNAs (20-48, hVEGFR1-25-20 to hVEGFR1-25-48 siRNA in Table 5) may stimulate the expression of soluble hVEGFR1 mRNA. HUVEC cells were transfected with 10 nM of siRNA and assayed at 48 hours post transfection for the levels of hVEGFR1 mRNAs, using a quantitative RT-PCR assay with a primer set specific for full-length hVEGFR1 mRNA (black bars) or a primer set for both the soluble and full-length hVEGFR1 mRNA (gray bars). 20-48: hVEGFR1-25-20 to hVEGFR1-25-48 siRNA in Table 5; Mock: mock transfection; Ctrl: negative control siRNA. Data were presented as mean+/−standard deviation.

FIGS. 7A and 7B show the nucleotide sequence of human VEGFR1 mRNA (GenBank Accession No. AF063657; SEQ ID NO: 197).

FIG. 8 shows the nucleotide sequence of human soluble VEGFR1 mRNA (GenBank Accession No. U01134; SEQ ID NO: 198).

FIGS. 9A and 9B show the nucleotide sequence of mouse VEGFR1 mRNA (GenBank Accession No. NM_{—}010228.2; SEQ ID NO: 199).

FIG. 10 is a schematic showing the structure and composition of the PolyTran™. PolyTran™ is a synthetic biodegradable cationic branched polypeptide. The positively charged PolyTran™ polypeptide serves as a carrier and condenser for the negatively charged siRNA. “R” disclosed as SEQ ID NO: 205.

### DETAILED DESCRIPTION OF THE INVENTION

The invention provides compositions and methods for treatment of diseases with unwanted neovascularization (NV) or angiogenesis, often an abnormal or excessive proliferation and growth of blood vessels. Since NV also can be a normal biological process, inhibition of unwanted NV is preferably accomplished with selectivity for a pathological tissue, which preferably requires selective delivery of therapeutic molecules to the pathological tissue using targeted nanoparticles. The present invention provides compositions and methods to control angiogenesis through selective inhibition of the VEGF biochemical pathway by nucleic acid molecules that induce RNA interference (RNAi), including inhibition of VEGF pathway gene expression and inhibition localized at pathological angiogenic tissues. In one embodiment, the invention provides nucleic acid molecules that inhibit VEGFR1 gene expression. The present invention also provides compositions of and methods for using synthetic nucleic acid delivery vehicles comprising polymer conjugates and further comprising nucleic acid molecules that induce RNAi.

The invention is described here in detail, but one skilled in the art will appreciate the full extent of the invention.

### DEFINITIONS

As used herein, “oligonucleotides” and similar terms based on this refers to oligonucleotides composed of naturally occurring nucleotides as well as to oligonucleotides composed of non-naturally occurring synthetic or modified nucleotides. Oligonucleotides may be 10 or more nucleotides in length, or 15, or 16, or 17, or 18, or 19, or 20 or more nucleotides in length, or 21, or 22, or 23, or 24 or more nucleotides in length, or 25, or 26, or 27, or 28 or 29, or 30 or more nucleotides in length, 35 or more, 40 or more, 45 or more, up to about 50, nucleotides in length.

An oligonucleotide that is an siRNA may have any number of nucleotides between 15 and 30 nucleotides. In many embodiments an siRNA may have any number of nucleotides between 19 and 27 nucleotides.

The term “antisense strand” refers to a nucleic acid strand that is substantially complementary to a section of about 10-50 nucleotides (for example, about 15-30, 16-25, 17-24, 18-23, or 19-22 nucleotides) of the mRNA sequence of the gene targeted for reduction of expression. The antisense strand (or first strand) has a sequence sufficiently complementary to the targeted mRNA sequence to induce destruction of the targeted mRNA by the RNAi process. The term “sense strand” or “second strand” refers to a nucleic acid strand that is substantially complementary to the “antisense strand” or “first strand”.

The term “VEGF” refers to total VEGF, unless otherwise specified or apparent from context.

### Nucleic Acid Molecules for VEGFR1 Gene Modulation

The present invention provides nucleic acid molecules for targeting and modulating VEGFR1 gene expression by RNAi. Exemplary siRNA sequences of the invention targeting the VEGFR1 gene are shown in Tables 1-5. (For all sequences listed in Tables 1-5, the “Start” position is labeled such that the “A” of the ATG codon is considered to be position 1.)

In one embodiment, the present invention provides nucleic acid molecules that result in a reduction in total or full-length (also referred to as “membrane-bound”) VEGFR1 mRNA or protein levels (also referred to as a “knockdown”) of at least 50%, 60%, 70%, 80%, 85%, 90%, 95, 96, 97, 98, 99 or 100% relative to the expression level in the absence of the nucleic acid molecule. In another embodiment, the nucleic acid molecules of the invention may increase the expression of soluble VEGFR1 mRNA or protein levels. The increase in expression of soluble VEGFR1 mRNA or protein levels may be at least 1.5-fold, at least 2-fold, at least 3-fold, at least 4-fold, at least 5-fold, at least 6-fold, at least 7-fold, at least 8-fold, at least 9-fold, or at least 10-fold relative to the expression level in the absence of the nucleic acid molecule. The nucleic acid molecules of the invention may reduce expression of VEGFR1 protein to about 50 pg/μg, 40 pg/μg, 30 pg/μg, 20 pg/μg, 15 pg/μg, 10 pg/μg, 7.5 pg/μg, 5 pg/μg, 2.5 pg/μg, 1 pg/μg or 0.5 pg/μg.

In one embodiment of the invention, the nucleic acid molecules reduce the expression of full-length VEGFR1 mRNA or protein levels while not affecting the expression of soluble VEGFR1 mRNA or protein levels. In another embodiment of the invention, the nucleic acid molecules increase the expression of total VEGFR1 mRNA or protein levels while increasing the expression of soluble VEGFR1 mRNA or protein levels. In another embodiment of the invention, the nucleic acid molecules decrease the expression of total VEGFR1 mRNA or protein levels while increasing the expression of soluble VEGFR1 mRNA or protein levels. In a preferred embodiment, the nucleic acid molecules reduce the expression of full-length VEGFR1 mRNA or protein levels while increasing the expression of soluble VEGFR1 mRNA or protein levels.

The modulation of total, full-length and/or soluble VEGFR1 may result up to 24 hours, up to 36 hours, up to 48 hours, up to 60 hours, up to 72 hours, up to 96 hours post administration of the nucleic acid molecules, or longer. In certain embodiments, the nucleic acid molecules that result in this modulation of gene expression may be administered at 30 nM, 25 nM, 20 nM, 15 nM, 12 nM, 10 nM, 7.5 nM, 5 nM, 2 nM, 1 nM, 0.75 nM, 0.5 nM, or 0.2 nM quantities.

The nucleic acid molecules of the invention may be dsRNA or ssRNA. In a preferred embodiment of the invention, the nucleic acid molecules are siRNA. The nucleic acid molecules may comprise 15-50, 15-30, 19-27, 19, 20, 21, 22, 23, 24 or 25 nucleotides. The nucleic acid molecules may comprise 10 or more nucleotides, or 15, or 16, or 17, or 18, or 19, or 20 or more nucleotides, or 21, or 22, or 23, or 24 or more nucleotides, or 25, or 26, or 27, or 28 or 29, or 30 or more nucleotides, 35 or more, 40 or more, 45 or more, or 50 or more nucleotides.

The nucleic acid molecules may comprise 5′- or 3′-single-stranded overhangs. The nucleic acid molecules may have two blunt ends, or two sticky ends, or one blunt end with one sticky end. The single-stranded overhang nucleotides of a sticky end can range from one to four or more. In a certain embodiment, the nucleic acid molecules are blunt-ended. In a preferred embodiment, the nucleic acid molecule is a double-stranded siRNA of 25 nucleotides with blunt ends.

In some embodiments, the nucleic acid molecules of the invention target both a human mRNA as well as the homologous or analogous mRNA in other non-human mammalian species such as primates, mice, or rats.

In one embodiment, the invention provides an antisense nucleic acid molecule for targeting VEGFR1, wherein the antisense nucleic acid comprises a sequence that is complementary to a sense strand selected from the group consisting of SEQ ID NOs: 129-196. In a further embodiment, the invention provides an antisense nucleic acid molecule for targeting VEGFR1, wherein the antisense nucleic acid targets a nucleotide sequence in the VEGFR1 mRNA comprising a nucleotide sequence selected from the group consisting of SEQ ID NOs: 24, 49, 50, 51, 84, 85, 86, 88, 89, 100, 104, 105, 184, 186, 188, 192, 193, 194, and 196.

TABLE 1

Candidate siRNAs targeting both soluble and membrane-bound
hVEGFR1

| | | | | |
|---|---|---|---|---|
| | | | | Sense |
| | | | | strand |
| | | | | SEQ ID |
| No. | Start | siRNA sequence (sense strand/antisense strand) | GC % | NO |

| | | | | |
|---|---|---|---|---|
| 1 | 173 | 5′-CCCAUAAAUGGUCUUUGCCUGAAAU-3′ | 40.0 | 129 |
| | | 3′-GGGUAUUUACCAGAAACGGACUUUA-5′ | | |
| 2 | 222 | 5′-GAGCAUAACUAAAUCUGCCUGUGGA-3′ | 44.0 | 130 |
| | | 3′-CUCGUAUUGAUUUAGACGGACACCU-5′ | | |
| 3 | 252 | 5′-UGGCAAACAAUUCUGCAGUACUUUA-3′ | 36.0 | 131 |
| | | 3′-ACCGUUUGUUAAGACGUCAUGAAAU-5′ | | |
| 4 | 253 | 5′-GGCAAACAAUUCUGCAGUACUUUAA-3′ | 36.0 | 132 |
| | | 3′-ACCGUUUGUUAAGACGUCAUGAAAU-5′ | | |
| 5 | 287 | 5′-CAGCUCAAGCAAACCACACUGGCUU-3′ | 52.0 | 133 |
| | | 3′-GUCGAGUUCGUUUGGUGUGACCGAA-5′ | | |
| 6 | 315 | 5′-CAGCUGCAAAUAUCUAGCUGUACCU-3′ | 44.0 | 134 |
| | | 3′-GUCGACGUUUAUAGAUCGACAUGGA-5′ | | |
| 7 | 321 | 5′-CAAAUAUCUAGCUGUACCUACUUCA-3′ | 36.0 | 135 |
| | | 3′-GUUUAUAGAUCGACAUGGAUGAAGU-5′ | | |
| 8 | 351 | 5′-GAAGGAAACAGAAUCUGCAAUCUAU-3′ | 36.0 | 136 |
| | | 3′-CUUCCUUUGUCUUAGACGUUAGAUA-5′ | | |
| 9 | 392 | 5′-CAGGUAGACCUUUCGUAGAGAUGUA-3′ | 44.0 | 137 |
| | | 3′-GUCCAUCUGGAAAGCAUCUCUACAU-5′ | | |
| 10 | 443 | 5′-UGACUGAAGGAAGGGAGCUCGUCAU-3′ | 52.0 | 138 |
| | | 3′-ACUGACUUCCUUCCCUCGAGCAGUA-5′ | | |
| 11 | 600 | 5′-AGAAAUAGGGCUUCUGACCUGUGAA-3′ | 44.0 | 139 |
| | | 3′-UCUUUAUCCCGAAGACUGGACACUU-5′ | | |
| 12 | 622 | 5′-GAAGCAACAGUCAAUGGGCAUUUGU-3′ | 44.0 | 140 |
| | | 3′-CUUCGUUGUCAGUUACCCGUAAACA-5′ | | |
| 13 | 625 | 5′-GCAACAGUCAAUGGGCAUUUGUAUA-3′ | 40.0 | 141 |
| | | 3′-CGUUGUCAGUUACCCGUAAACAUAU-5′ | | |
| 14 | 626 | 5′-CAACAGUCAAUGGGCAUUUGUAUAA-3′ | 36.0 | 142 |
| | | 3′-CGUUGUCAGUUACCCGUAAACAUAU-5′ | | |
| 15 | 715 | 5′-CCAGUCAAAUUACUUAGAGGCCAUA-3′ | 40.0 | 143 |
| | | 3′-GGUCAGUUUAAUGAAUCUCCGGUAU-5′ | | |
| 16 | 719 | 5′-UCAAAUUACUUAGAGGCCAUACUCU-3′ | 36.0 | 144 |
| | | 3′-AGUUUAAUGAAUCUCCGGUAUGAGA-5′ | | |
| 17 | 720 | 5′-CAAAUUACUUAGAGGCCAUACUCUU-3′ | 36.0 | 145 |
| | | 3′-GUUUAAUGAAUCUCCGGUAUGAGAA-5′ | | |
| 18 | 733 | 5′-GGCCAUACUCUUGUCCUCAAUUGUA-3′ | 44.0 | 146 |
| | | 3′-CCGGUAUGAGAACAGGAGUUAACAU-5′ | | |
| 19 | 744 | 5′-UGUCCUCAAUUGUACUGCUACCACU-3′ | 44.0 | 147 |
| | | 3′-ACAGGAGUUAACAUGACGAUGGUGA-5′ | | |
| 20 | 764 | 5′-CCACUCCCUUGAACACGAGAGUUCA-3′ | 52.0 | 148 |
| | | 3′-GGUGAGGGAACUUGUGCUCUCAAGU-5′ | | |
| 21 | 1050 | 5′-GCGGUCUUACCGGCUCUCUAUGAAA-3′ | 52.0 | 149 |
| | | 3′-CGCCAGAAUGGCCGAGAGAUACUUU-5′ | | |
| 22 | 1086 | 5′-UCCCUCGCCGGAAGUUGUAUGGUUA-3′ | 52.0 | 150 |
| | | 3′-AGGGAGCGGCCUUCAACAUACCAAU-5′ | | |
| 23 | 1125 | 5′-UGCGACUGAGAAAUCUGCUCGCUAU-3′ | 48.0 | 151 |
| | | 3′-ACGCUGACUCUUUAGACGAGCGAUA-5′ | | |
| 24 | 1147 | 5′-UAUUUGACUCGUGGCUACUCGUUAA-3′ | 40.0 | 152 |
| | | 3′-AUAAACUGAGCACCGAUGAGCAAUU-5′ | | |
| 25 | 1151 | 5′-UGACUCGUGGCUACUCGUUAAUUAU-3′ | 40.0 | 153 |
| | | 3′-ACUGAGCACCGAUGAGCAAUUAAUA-5′ | | |
| 26 | 1201 | 5′-GGGAAUUAUACAAUCUUGCUGAGCA-3′ | 40.0 | 154 |
| | | 3′-CCCUUAAUAUGUUAGAACGACUCGU-5′ | | |
| 27 | 1254 | 5′-CACUGCCACUCUAAUUGUCAAUGUG-3′ | 44.0 | 155 |
| | | 3′-GUGACGGUGAGAUUAACAGUUACAC-5′ | | |
| 28 | 1339 | 5′-GGCAGCAGACAAAUCCUGACUUGUA-3′ | 48.0 | 156 |
| | | 3′-CCGUCGUCUGUUUAGGACUGAACAU-5′ | | |
| 29 | 1344 | 5′-CAGACAAAUCCUGACUUGUACCGCA-3′ | 48.0 | 157 |
| | | 3′-GUCUGUUUAGGACUGAACAUGGCGU-5′ | | |
| 30 | 1576 | 5′-GACUCUAGAAUUUCUGGAAUCUACA-3′ | 36.0 | 158 |
| | | 3′-CUGAGAUCUUAAAGACCUUAGAUGU-5′ | | |
| 31 | 1653 | 5′-UAUCACAGAUGUGCCAAAUGGGUUU-3′ | 40.0 | 159 |
| | | 3′-AUAGUGUCUACACGGUUUACCCAAA-5′ | | |
| 32 | 1660 | 5′-GAUGUGCCAAAUGGGUUUCAUGUUA-3′ | 40.0 | 160 |
| | | 3′-CUACACGGUUUACCCAAAGUACAAU-5′ | | |
| 33 | 1826 | 5′-UGGCCAUCACUAAGGAGCACUCCAU-3′ | 52.0 | 161 |
| | | 3′-ACCGGUAGUGAUUCCUCGUGAGGUA-5′ | | |
| 34 | 1830 | 5′-CAUCACUAAGGAGCACUCCAUCACU-3′ | 48.0 | 162 |
| | | 3′-GUAGUGAUUCCUCGUGAGGUAGUGA-5′ | | |
| 35 | 1833 | 5′-CACUAAGGAGCACUCCAUCACUCUU-3′ | 48.0 | 163 |
| | | 3′-GUGAUUCCUCGUGAGGUAGUGAGAA-5′ | | |
| 36 | 1847 | 5′-CCAUCACUCUUAAUCUUACCAUCAU-3′ | 36.0 | 164 |
| | | 3′-GGUAGUGAGAAUUAGAAUGGUAGUA-5′ | | |
| 37 | 1848 | 5′-CAUCACUCUUAAUCUUACCAUCAUG-3′ | 36.0 | 165 |
| | | 3′-GGUAGUGAGAAUUAGAAUGGUAGUA-5′ | | |
| 38 | 1900 | 5′-UAUGCCUGCAGAGCCAGGAAUGUAU-3′ | 48.0 | 166 |
| | | 3′-AUACGGACGUCUCGGUCCUUACAUA-5′ | | |

TABLE 2

Candidate siRNAs targeting membrane-bound hVEGFR1 but not soluble
hVEGFR1

| | | | | |
|---|---|---|---|---|
| | | | | Sense strand |
| No. | Start | siRNA sequence (sense strand/antisense strand) | GC % | SEQ ID NO |

| | | | | |
|---|---|---|---|---|
| 1 | 1973 | 5′-AGGAAGCACCAUACCUCCUGCGAAA-3′ | 52.0 | 167 |
| | | 3′-UCCUUCGUGGUAUGGAGGACGCUUU-5′ | | |
| 2 | 2145 | 5′-GCUGUUUAUUGAAAGAGUCACAGAA-3′ | 36.0 | 49 |
| | | 3′-CGACAAAUAACUUUCUCAGUGUCUU-5′ | | |
| 3 | 2205 | 5′-CCAGAAGGGCUCUGUGGAAAGUUCA-3′ | 52.0 | 168 |
| | | 3′-GGUCUUCCCGAGACACCUUUCAAGU-5′ | | |
| 4 | 2206 | 5′-CAGAAGGGCUCUGUGGAAAGUUCAG-3′ | 52.0 | 169 |
| | | 3′-GUCUUCCCGAGACACCUUUCAAGUC-5′ | | |
| 5 | 2211 | 5′-GGGCUCUGUGGAAAGUUCAGCAUAC-3′ | 52.0 | 170 |
| | | 3′-CCCGAGACACCUUUCAAGUCGUAUG-5′ | | |
| 6 | 2249 | 5′-GAACCUCGGACAAGUCUAAUCUGGA-3′ | 48.0 | 171 |
| | | 3′-CUUGGAGCCUGUUCAGAUUAGACCU-5′ | | |
| 7 | 2274 | 5′-GCUGAUCACUCUAACAUGCACCUGU-3′ | 48.0 | 172 |
| | | 3′-CGACUAGUGAGAUUGUACGUGGACA-5′ | | |
| 8 | 2305 | 5′-GCGACUCUCUUCUGGCUCCUAUUAA-3′ | 48.0 | 173 |
| | | 3′-CGCUGAGAGAAGACCGAGGAUAAUU-5′ | | |
| 9 | 2382 | 5′-CCUAUCAAUUAUAAUGGACCCAGAU-3′ | 36.0 | 174 |
| | | 3′-GGAUAGUUAAUAUUACCUGGGUCUA-5′ | | |
| 10 | 2528 | 5′-AAGCAUCAGCAUUUGGCAUUAAGAA-3′ | 36.0 | 175 |
| | | 3′-UUCGUAGUCGUAAACCGUAAUUCUU-5′ | | |
| 11 | 2657 | 5′-GCCACCAUCUGAACGUGGUUAACCU-3′ | 52.0 | 176 |
| | | 3′-CGGUGGUAGACUUGCACCAAUUGGA-5′ | | |
| 12 | 2708 | 5′-GGCCUCUGAUGGUGAUUGUUGAAUA-3′ | 44.0 | 177 |
| | | 3′-CCGGAGACUACCACUAACAACUUAU-5′ | | |
| 13 | 2710 | 5′-CCUCUGAUGGUGAUUGUUGAAUACU-3′ | 40.0 | 178 |
| | | 3′-GGAGACUACCACUAACAACUUAUGA-5′ | | |
| 14 | 2715 | 5′-GAUGGUGAUUGUUGAAUACUGCAAA-3′ | 36.0 | 179 |
| | | 3′-CUACCACUAACAACUUAUGACGUUU-5′ | | |
| 15 | 2759 | 5′-ACCUCAAGAGCAAACGUGACUUAUU-3′ | 40.0 | 51 |
| | | 3′-UGGAGUUCUCGUUUGCACUGAAUAA-5′ | | |
| 16 | 2760 | 5′-CCUCAAGAGCAAACGUGACUUAUUU-3′ | 40.0 | 43 |
| | | 3′-GGAGUUCUCGUUUGCACUGAAUAAA-5′ | | |
| 17 | 2901 | 5′-GAGCUCCGGCUUUCAGGAAGAUAAA-3′ | 48.0 | 180 |
| | | 3′-CUCGAGGCCGAAAGUCCUUCUAUUU-5′ | | |
| 18 | 3027 | 5′-CAUGGAGUUCCUGUCUUCCAGAAAG-3′ | 48.0 | 181 |
| | | 3′-GUACCUCAAGGACAGAAGGUCUUUC-5′ | | |
| 19 | 3031 | 5′-GAGUUCCUGUCUUCCAGAAAGUGCA-3′ | 48.0 | 182 |
| | | 3′-CUCAAGGACAGAAGGUCUUUCACGU-5′ | | |
| 20 | 3347 | 5′-GCAUGAGGAUGAGAGCUCCUGAGUA-3′ | 52.0 | 183 |
| | | 3′-CGUACUCCUACUCUCGAGGACUCAU-5′ | | |
| 21 | 3357 | 5′-GAGAGCUCCUGAGUACUCUACUCCU-3′ | 52.0 | 184 |
| | | 3′-CUCUCGAGGACUCAUGAGAUGAGGA-5′ | | |
| 22 | 3431 | 5′-GGCCAAGAUUUGCAGAACUUGUGGA-3′ | 48.0 | 185 |
| | | 3′-CCGGUUCUAAACGUCUUGAACACCU-5′ | | |
| 23 | 3458 | 5′-AACUAGGUGAUUUGCUUCAAGCAAA-3′ | 36.0 | 186 |
| | | 3′-UUGAUCCACUAAACGAAGUUCGUUU-5′ | | |
| 24 | 3462 | 5′-AGGUGAUUUGCUUCAAGCAAAUGUA-3′ | 36.0 | 187 |
| | | 3′-UCCACUAAACGAAGUUCGUUUACAU-5′ | | |
| 25 | 3527 | 5′-UGACAGGAAAUAGUGGGUUUACAUA-3′ | 36.0 | 188 |
| | | 3′-ACUGUCCUUUAUCACCCAAAUGUAU-5′ | | |
| 26 | 3532 | 5′-GGAAAUAGUGGGUUUACAUACUCAA-3′ | 36.0 | 189 |
| | | 3′-CCUUUAUCACCCAAAUGUAUGAGUU-5′ | | |
| 27 | 3585 | 5′-GGAAAGUAUUUCAGCUCCGAAGUUU-3′ | 40.0 | 119 |
| | | 3′-CCUUUCAUAAAGUCGAGGCUUCAAA-5′ | | |
| 28 | 3798 | 5′-GGCCUCGCUCAAGAUUGACUUGAGA-3′ | 52.0 | 190 |
| | | 3′-CCGGAGCGAGUUCUAACUGAACUCU-5′ | | |
| 29 | 3802 | 5′-UCGCUCAAGAUUGACUUGAGAGUAA-3′ | 40.0 | 191 |
| | | 3′-AGCGAGUUCUAACUGAACUCUCAUU-5′ | | |
| 30 | 3810 | 5′-GAUUGACUUGAGAGUAACCAGUAAA-3′ | 36.0 | 192 |
| | | 3′-CUAACUGAACUCUCAUUGGUCAUUU-5′ | | |
| 31 | 3974 | 5′-CAGACUACAACUCGGUGGUCCUGUA-3′ | 52.0 | 193 |
| | | 3′-GUCUGAUGUUGAGCCACCAGGACAU-5′ | | |
| 32 | 3976 | 5′-GACUACAACUCGGUGGUCCUGUACU-3′ | 52.0 | 194 |
| | | 3′-CUGAUGUUGAGCCACCAGGACAUGA-5′ | | |

TABLE 3
Candidate siRNAs targeting both human and mouse VEGFR1

| | | | | |
|---|---|---|---|---|
| | | | | Sense strand |
| No. | Start | siRNA sequence (sense strand/antisense strand) | GC % | SEQ ID NO |

| | | | | |
|---|---|---|---|---|
| 1 | 95 | 5′-CUGAACUGAGUUUAAAAGGCACCCA-3′ | 44.0 | 106 |
| | | 3′-GACUUGACUCAAAUUUUCCGUGGGU-5′ | | |
| 2 | 97 | 5′-GAACUGAGUUUAAAAGGCACCCAGC-3′ | 48.0 | 107 |
| | | 3′-CUUGACUCAAAUUUUCCGUGGGUCG-5′ | | |
| 3 | 2139 | 5′-CAGCACGCUGUUUAUUGAAAGAGUC-3′ | 44.0 | 195 |
| | | 3′-GUCGUGCGACAAAUAACUUUCUCAG-5′ | | |
| 4 | 2141 | 5′-GCACGCUGUUUAUUGAAAGAGUCAC-3′ | 44.0 | 196 |
| | | 3′-CGUGCGACAAAUAACUUUCUCAGUG-5′ | | |
| 5 | 2142 | 5′-CACGCUGUUUAUUGAAAGAGUCACA-3′ | 36.0 | 100 |
| | | 3′-GUGCGACAAAUAACUUUCUCAGUGU-5′ | | |
| 6 | 2144 | 5′-CGCUGUUUAUUGAAAGAGUCACAGA-3′ | 36.0 | 50 |
| | | 3′-GCGACAAAUAACUUUCUCAGUGUCU-5′ | | |
| 7 | 2753 | 5′-CCAACUACCUCAAGAGCAAACGUGA-3′ | 48.0 | 24 |
| | | 3′-UGGAGUUCUCGUUUGCACUGAAUAA-5′ | | |
| 8 | 2757 | 5′-CUACCUCAAGAGCAAACGUGACUUA-3′ | 44.0 | 25 |
| | | 3′-GAUGGAGUUCUCGUUUGCACUGAAU-5′ | | |
| 9 | 2759 | 5′-ACCUCAAGAGCAAACGUGACUUAUU-3′ | 40.0 | 51 |
| | | 3′-UGGAGUUCUCGUUUGCACUGAAUAA-5′ | | |
| 10 | 3660 | 5′-GAGCCUGGAAAGAAUCAAAACCUUU-3′ | 40.0 | 104 |
| | | 3′-CUCGGACCUUUCUUAGUUUUGGAAA-5′ | | |
| 11 | 3662 | 5′-GCCUGGAAAGAAUCAAAACCUUUGA-3′ | 40.0 | 105 |
| | | 3′-CGGACCUUUCUUAGUUUUGGAAACU-5′ | | |

The efficacy of RNAi-inducing nucleic acid molecules of the invention, particularly double-stranded nucleic acid molecules such as siRNA, may be improved by methods described in U.S. Patent Application Publication Nos. 2005/0186586, 2005/0181382, 2005/0037988, and 2006/0134787, which are herein incorporated by reference in their entirety. A “guide strand” is a strand of an RNAi agent that enters the RISC and directs degradation of the targeted mRNA. The efficacy of the siRNA molecule in acting as a guide strand can be enhanced by increasing the asymmetry of the molecule. In brief, the ability of the siRNA molecule to act as a guide strand in RNAi can be increased by lessening the base pair strength between the 5′ end of the first strand and the 3′ end of a second strand of the duplex as compared to the base pair strength between the 3′ end of the first strand and the 5′ end of the second strand. In one embodiment of the invention, the ability of the siRNA molecule to act as a guide strand in RNAi can be increased by lessening the base pair strength between the antisense strand 5′ end and the sense strand 3′ end as compared to the base pair strength between the antisense strand 3′ end and the sense strand 5′ end.

The base pair strength can be lessened by decreasing the number of G:C base pairs or inserting one or more mismatched base pairs. Examples of mismatched base pairs include G:A, C:A, C:U, G:G, A:A, C:C, U:U, C:T, and U:T. Inserting wobble base pairs such as G:U, or G:T between the 5′ end of the first or antisense strand and the 3′ end of the second or sense strand also lessens the base pair strength. In one embodiment of the invention, one or more of these methods is combinded to lessen the base pair strength and increase the efficacy of the siRNA molecules of the invention.

In certain embodiments, the base pair strength is lessened by incorporation of at least one base pair comprising a rare nucleotide such as inosine, 1-methyl inosine, pseudouridine, 5,6-dihydrouridine, ribothymidine, 2N-methylguanosine and 2,2N,N-dimethylguanosine; or a modified nucleotide, such as 2-amino-G, 2-amino-A, 2,6-diamino-G, and 2,6-diamino-A.

### Chemical Modification

Chemical modification may be useful in some embodiments of the invention to increase stability of the nucleic acid molecule or to reduce cytokine production. Incorporation of non-naturally occurring chemical analogues, such as 2′-O-Methyl ribose analogues of RNA, DNA, LNA and RNA chimeric oligonucleotides, and other chemical analogues of nucleic acid oligonucleotides, is one type of possible chemical modification. Possible modifications also include the addition of flanking sequences at the 5′ and/or 3′ ends; the use of phosphorothioate, methylphosphonate, sulfone, sulfate, ketyl, phosphorodithioate, phosphoramidate, phosphate esters, or 2′O-methyl rather than phosphodiester linkages in the backbone; and/or the inclusion of non-traditional bases, as well as acetyl-methyl-, thio- and other modified forms of adenine, cytidine, guanine, thymine, and uridine. Non-traditional nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5″-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, beta-D-mannosylqueosine, uridine-5-oxyacetic acid, 2-thiocytidine, threonine derivatives and others (see, for example, Molecular Therapy, 2007; 15:1663-1669, incorporated herein by reference in its entirety). These polynucleotide variants may be modified such that the activity of the nucleic acid molecule is not substantially decreased.

In certain embodiments, oligonucleotides of the invention may be 2′-O-substituted oligonucleotides, as described in U.S. Pat. Nos. 5,623,065, 5,856,455, 5,955,589, 6,146,829, and 6,326,199, herein incorporated by reference in their entirety, in which 2′ substituted nucleotides are introduced within an oligonucleotide to induce increased binding of the oligonucleotide to a complementary target strand while allowing expression of RNase H activity to destroy the targeted strand. See also, Sproat, B. S., et al., Nucleic Acids Research, 1990; 18:41, incorporated herein by reference in its entirety. Nucleic acid molecules comprising 2′-O-methyl and ethyl nucleotides are also encompassed by the invention.

A number of groups have taught the preparation of other 2′-O-alkyl guanosines. Gladkaya, et al., Khim. Prir. Soedin., 1989; 4:568, incorporated herein by reference in its entirety, discloses N₁-methyl-2′-O-(tetrahydropyran-2-yl) and 2′-O-methyl guanosine and Hansske, et al., Tetrahedron, 1984; 40:125, incorporated herein by reference in its entirety, discloses a 2′-O-methylthiomethylguanosine. The 2′-O-methylthiomethyl derivative of 2,6-diaminopurine riboside has also been reported. Sproat, et al., Nucleic Acids Research, 1991; 19:733, incorporated herein by reference in its entirety, teaches the preparation of 2′-O-allyl-guanosine. Iribarren, et al., Proc. Natl. Acad. Sci., 1990; 87:7747, incorporated herein by reference in its entirety, also studied 2′-O-allyl oligoribonucleotides. In certain embodiments, the nucleic acid molecules of the invention comprise 2′-O-methyl-, 2′-O-allyl-, and 2′-O-dimethylallyl-substituted nucleotides.

In certain embodiments, at least one of the 2′-deoxyribofuranosyl moiety of at least one of the nucleosides of an oligonucleotide is modified. A halo, alkoxy, aminoalkoxy, alkyl, azido, or amino group may be added. For example, F, CN, CF₃, OCF₃, OCN, O-alkyl, S-alkyl, SMe, SO₂ Me, ONO₂, NO₂, NH₃, NH₂, NH-alkyl, OCH₂ CH═CH₂ (allyloxy), OCH₃═CH₂, OCCH, where alkyl is a straight or branched chain of C₁ to C₂₀, with unsaturation within the carbon chain. PCT/US91/00243, application Ser. No. 463,358, and application Ser. No. 566,977, disclose that incorporation of, for example, a 2′-O-methyl, 2′-O-ethyl, 2′-O-propyl, 2′-O-allyl, 2′-O-aminoalkyl or 2′-deoxy-2′-fluoro groups on the nucleosides of an oligonucleotide enhance the hybridization properties of the oligonucleotide. The nucleic acid molecules of the invention can be augmented to further include either or both a phosphorothioate backbone or a 2′-O—C₁ C₂₀-alkyl (e.g., 2′-O-methyl, 2′-O-ethyl, 2′-O-propyl), 2′-O—C₂ C₂₀-alkenyl (e.g., 2′-O-allyl), 2′-O—C₂ C₂₀-alkynyl, 2′-S—C₁ C₂₀-alkyl, 2′-S—C₂ C₂₀-alkenyl, 2′-S—C₂ C₂₀-alkynyl, 2′-NH—C₁ C₂₀-alkyl (2′-O-aminoalkyl), 2′-NH—C₂ C₂₀-alkenyl, 2′-NH—C₂ C₂₀-alkynyl or 2′-deoxy-2′-fluoro group for increased stability. See, e.g., U.S. Pat. No. 5,506,351, herein incorporated by reference in its entirety.

Exemplary modified nucleotides can be found in U.S. Pat. Nos. 7,101,993, 7,056,896, 6,911,540, 7,015,315, 5,872,232, and 5,587,469, herein incorporated by reference in their entirety.

### Combined VEGF Pathway Gene Modulation

One aspect of the present invention is to combine antisense nucleic acid molecules, such as siRNAs, so as to achieve specific and selective inhibition of VEGFR1 and multiple other VEGF pathway genes and as a result inhibit NV disease and provide a better clinical benefit. The present invention provides for many combinations of siRNA targets, including combinations of VEGFR1 with either VEGF or VEGFR2. Exemplary siRNA sequences targeting VEGF, VEGFR1, and VEGFR2 mRNAs are listed in Tables 1-5 and 7-11. In one embodiment, the invention provides a combination of siRNAs targeting VEGF, VEGFR1, and VEGFR2. The present invention also provides for combinations of siRNAs targeting one or more sequences within the same gene in the VEGF pathway.

Another embodiment of the invention is a combination of siRNA targeting VEGFR1 and one or more genes selected from the group consisting of VEGF, VEGFR2, PDGF and its receptors, EGF and its receptors, downstream signaling factors including RAF and AKT, and transcription factors including NFκB. Exemplary siRNA sequences targeting PDGFR and EGFR can be found in U.S. Patent Application Publication Nos. 2008/0220027 and 2008/0153771 and PCT/US2008/007672, which are incorporated herein by reference in their entirety. Yet another embodiment of the invention is a combination of siRNA inhibiting VEGF and its receptors and their downstream genes.

The nucleic acid molecules of the invention can be combined as a therapeutic for the treatment of NV-related disease. In one embodiment of the present invention they can be mixed together as a cocktail and in another embodiment they can be administered sequentially by the same route or by different routes and formulations and in yet another embodiment some can be administered as a cocktail and some administered sequentially. In one embodiment, multiple siRNA oligonucleotides can be formulated in a single preparation such as a nanoparticle preparation. Other combinations of nucleic acid molecules and methods for their combination will be understood by one skilled in the art to achieve treatment of NV-related diseases.

### Therapeutic Methods of Use

The present invention also provides methods for the treatment of angiogenesis- or NV-related diseases and conditions in a subject. In some embodiments, the present invention provides a method of treating a subject afflicted with a disease or condition associated with undesired angiogenesis comprising administering to the subject siRNA molecules that target VEGFR1 so that expression of total VEGFR1 is decreased. In another embodiment, the present invention provides a method of treating a subject afflicted with a disease or condition associated with undesired angiogenesis comprising administering to the subject siRNA molecules that target VEGFR1 so that expression of full-length VEGFR1 is decreased while the expression of soluble VEGFR1 is not affected or increased. In one aspect of the invention, such siRNA molecules comprise a nucleotide sequence that is complementary to a sense strand selected from the group consisting of SEQ ID NOs: 24, 25, 43, 49, 50, 51, 83, 84, 85, 86, 87, 88, 89, 100, 104, 105, 173, 180, 181, 182, 183, 184, 186, 187, 188, 192, 193, 194, and 196. In a preferred embodiment, the present invention provides a method of treating a subject afflicted with a disease or condition associated with undesired angiogenesis comprising administering to the subject siRNA molecules that target VEGFR1 so that expression of full-length VEGFR1 is decreased while the expression of soluble VEGFR1 is increased. In this embodiment, such siRNA molecules comprise a nucleotide sequence that is complementary to a sense strand selected from the group consisting of SEQ ID NOs: 24, 49, 50, 51, 84, 85, 86, 88, 89, 100, 104, 105, 184, 186, 188, 192, 193, 194, and 196.

In some embodiments, the present invention provides a method of treating a subject afflicted with a disease or condition associated with undesired angiogenesis comprising administering to the subject siRNA molecules that target VEGFR1 and siRNA molecules that target VEGF so that expression of VEGFR1 and VEGF is decreased. In some embodiments, the present invention provides a method of treating a subject afflicted with a disease or condition associated with undesired angiogenesis comprising administering to the subject siRNA molecules that target VEGFR1 and siRNA molecules that target VEGFR2 so that expression of VEGFR1 and VEGFR2 is decreased. In further embodiments, the present invention provides a method of treating a subject afflicted with a disease or condition associated with undesired angiogenesis comprising administering to the subject siRNA molecules that target VEGFR1, siRNA molecules that target VEGF and siRNA molecules that target VEGFR2 so that expression of VEGFR1, VEGF and VEGFR2 is decreased.

The present invention also provides methods for the treatment of angiogenesis- or NV-related disease in a subject, including cancer, ocular disease, arthritis, and inflammatory diseases. The angiogenesis-related diseases include, but are not limited to, carcinoma, such as breast, ovarian, stomach, endometrial, salivary gland, lung, kidney, colon, colorectum, esophageal, thyroid, pancreatic, prostate and bladder carcinomas and other neoplastic diseases, such as melanoma, small cell lung cancer, non-small cell lung cancer, glioma, hepatocellular (liver) carcinoma, sarcoma, head and neck cancers, mesothelioma, biliary (cholangiocarcinoma), small bowel adenocarcinoma, pediatric malignancies and glioblastoma.

Antagonizing these molecules is expected to inhibit pathophysiological processes, and thereby act as a potent therapy for various angiogenesis-dependent diseases. Besides solid tumors and their metastases, haematologic malignancies, such as leukemias, lymphomas and multiple myeloma, are also angiogenesis-dependent. Excessive vascular growth contributes to numerous non-neoplastic disorders. These non-neoplastic angiogenesis-dependent diseases include: atherosclerosis, haemangioma, haemangioendothelioma, angiofibroma, vascular malformations (e.g. Hereditary Hemorrhagic Teleangiectasia (HHT), or Osler-Weber syndrome), warts, pyogenic granulomas, excessive hair growth, Kaposis' sarcoma, scar keloids, allergic oedema, psoriasis, dysfunctional uterine bleeding, follicular cysts, ovarian hyperstimulation, endometriosis, respiratory distress, ascites, peritoneal sclerosis in dialysis patients, adhesion formation result from abdominal surgery, obesity, rheumatoid arthritis, synovitis, osteomyelitis, pannus growth, osteophyte, hemophilic joints, inflammatory and infectious processes (e.g. hepatitis, pneumonia, glomerulonephritis), asthma, nasal polyps, liver regeneration, pulmonary hypertension, retinopathy of prematurity, diabetic retinopathy, age-related macular degeneration, leukomalacia, neovascular glaucoma, corneal graft neovascularization, trachoma, thyroiditis, thyroid enlargement, and lymphoproliferative disorders.

In one embodiment of the invention, the subject treated is a human.

### Compositions and Methods of Administration

In another aspect, this invention provides compositions comprising the nucleic acid molecules, including siRNA, of the invention. The siRNA of the composition may be targeted to mRNA from the VEGF pathway, specifically to the VEGFR1 gene. The compositions may comprise the nucleic acid molecules and a pharmaceutically acceptable carrier, for example, a saline solution or a buffered saline solution.

In certain embodiments, this invention provides “naked” nucleic acid molecules or nucleic acid molecules in a nucleic acid delivery vehicle. In embodiments comprising a nucleic acid delivery vehicle, the vehicle can be a naturally occurring vector, such as a viral vector, or synthetic vector, such as a liposome, polylysine, or a cationic polymer. In one embodiment, the composition may comprise the siRNA of the invention and a complex-forming agent, such as a cationic polymer. The composition may also comprise a hydrophilic polymer, such as polyethylene glycol (PEG). The cationic polymer may be a histidine-lysine (HK) copolymer or a polyethyleneimine.

In certain embodiments, the cationic polymer is an HK copolymer. In certain embodiments, the HK copolymer is synthesized from any appropriate combination of polyhistidine, polylysine, histidine and/or lysine. In certain embodiments, the HK copolymer is linear. In certain preferred embodiments, the HK copolymer is branched.

In certain preferred embodiments, the branched HK copolymer comprises a polypeptide backbone. The polypeptide backbone may comprise 1-10 amino acid residues, and preferably 2-5 amino acid residues.

In certain embodiments, the polypeptide backbone consists of lysine amino acid residues.

In certain embodiments, the number of branches on the branched HK copolymer is the number of backbone amino acid residues plus one. In certain embodiments, the branched HK copolymer contains 1-11 branches. In certain preferred embodiments, the branched HK copolymer contains 2-5 branches. In certain more preferred embodiments, the branched HK copolymer contains 4 branches.

In some embodiments, the branch of the branched HK copolymer comprises 10-100 amino acid residues. In certain preferred embodiments, the branch comprises 10-50 amino acid residues. In certain more preferred embodiments, the branch comprises 15-25 amino acid residues. In certain embodiments, the branch of the branched HK copolymer comprises at least 3 histidine amino acid residues in every subsegment of 5 amino acid residues. In certain other embodiments, the branch comprises at least 3 histidine amino acid residues in every subsegment of 4 amino acid residues. In certain other embodiments, the branch comprises at least 2 histidine amino acid residues in every subsegment of 3 amino acid residues. In certain other embodiments, the branch comprises at least 1 histidine amino acid residues in every subsegment of 2 amino acid residues.

In certain embodiments, at least 50% of the branch of the HK copolymer comprises units of the sequence KHHH (SEQ ID NO: 200). In certain preferred embodiments, at least 75% of the branch comprises units of the sequence KHHH (SEQ ID NO: 200).

In certain embodiments, the HK copolymer branch comprises an amino acid residue other than histidine or lysine. In certain preferred embodiments, the branch comprises a cysteine amino acid residue, wherein the cysteine is a N-terminal amino acid residue.

In certain embodiments, the HK copolymer has the structure

| | |
|---|---|
| (KHHHKHHHKHHHHKHHHK)₄-KKK. | (SEQ ID NO: 201) |

In certain other embodiments, the HK copolymer has the structure

| | |
|---|---|
| (CKHHHKHHHKHHHHKHHHK)₄-KKK. | (SEQ ID NO: 202) |

In a preferred embodiment, the HK copolymer is PolyTran™ and has the structure shown in FIG. 10.

Some suitable examples of HK copolymers can be found, for example, in U.S. Pat. Nos. 6,692,911, 7,070,807, and 7,163,695, which are incorporated herein by reference in their entirety.

In one embodiment, the compositions of the invention may comprise the siRNA of the invention and a complex-forming agent that is used to make a nanoparticle. The nanoparticle may optionally comprise a steric polymer and/or a targeting moiety. The targeting moiety may be a peptide, an antibody, or an antigen-binding portion. The targeting moiety may serve as a means for targeting vascular endothelial cells, such as a peptide comprising the sequence Arg-Gly-Asp (RGD). Such a peptide may be cyclic or linear. In one embodiment, this peptide is RGDFK (SEQ ID NO: 203). In a certain embodiment, this peptide is cyclo (RGD-D-FK (SEQ ID NO: 204)). In another embodiment, this peptide is ACRGDMFGCA (SEQ ID NO: 12).

The nucleic acid molecules, compositions, and therapeutic methods of the invention can be used alone or in combination with other therapeutic agents and modalities including targeted therapeutics and including VEGF pathway antagonists, such as monoclonal antibodies and small molecule inhibitors, and targeted therapeutics inhibiting EGF and its receptors, PDGF and its receptors, or MEK or Bcr-Abl, and other immunotherapeutic and chemotherapeutic agents, such as EGFR inhibitors VECTIBIX® (panitumumab) and TARCEVA® (erlotinib), Her-2-targeted therapy HERCEPTIN® (trastuzumab), or anti-angiogenesis drugs such as AVASTIN® (bevacizumab) and SUTENT® (sunitinib malate). The nucleic acid molecules, compositions, and methods also may be combined therapeutically with other treatment modalities including radiation, laser therapy, surgery and the like.

Methods of administration for the nucleic acids and compositions of the invention are known to those of ordinary skill in the art. Administration may be intravenous, intraperitoneal, intramuscular, intracavity, subcutaneous, cutaneous, or transdermal. In one embodiment, administration may be systemic. In a further embodiment, administration may be local. For example, the nucleic acid molecules of the invention may be delivered via direct injections into tumor tissue and directly into or near angiogenic tissue or tissue with undesirable neovasculature. For certain applications, the nucleic acid molecules and compositions may be administered with application of an electric field. In certain embodiments, this invention provides for administration of “naked” siRNA.

Exemplary animal models for testing administration of the nucleic acids, nucleic acid delivery vehicles, and compositions of the present invention, including systemic administration, can be found in WO 08/45576 and U.S. Patent Application Publications Nos. 2008/0220027 and 2008/0153771, incorporated herein by reference in their entirety.

### Preparation of Nanoparticles Containing Nucleic Acid Molecules Modulating Expression of VEGF Pathway Genes

One embodiment of the present invention provides compositions and methods for nanoparticle preparations of anti-VEGF pathway nucleic acid molecules, including siRNAs. The nanoparticles may comprise one or more of a histidine-lysine copolymer, polyethylene glycol, or polyethyleneimine. In one embodiment of the invention, RGD-mediated ligand-directed nanoparticles may be prepared. In one method for the manufacture of RGD-mediated tissue-targeted nanoparticles containing siRNA, the targeting ligand, an ROD-containing peptide, is conjugated to a steric polymer such as polyethylene glycol, or other polymers with similar properties. This ligand-steric polymer conjugate is further conjugated to a polycation such as polyethyleneimine or other effective material such as a histidine-lysine copolymer. The conjugation can be by covalent or non-covalent bonds and the covalent bonds can be non-cleavable or they can be cleavable such as by hydrolysis or by reducing agents. A solution comprising the polymer conjugate, or comprising a mixture of a polymer conjugate with other polymer, lipid, or micelle such as materials comprising a ligand or a steric polymer or fusogen, is mixed with a solution comprising the nucleic acid, in one embodiment an siRNA targeted against specific mRNA of interest, in desirable ratios to obtain nanoparticles that contain siRNA. Such ratios may produce nanoparticles of a desired size, stability, or other characteristics.

In one embodiment, nanoparticles are formed by nanoparticle self-assembly comprising mixing the polymer conjugate with excess polycation and the nucleic acid. Non-covalent electrostatic interactions between the negatively charged nucleic acid and the positively charged segment of the polymer conjugate drive the self-assembly process that leads to formation of nanoparticles. This process involves simple mixing of the solutions where one of the solutions containing the nucleic acid is added to another solution containing the polymer conjugate and excess polycation followed by or concurrently with stirring. In one embodiment, the ratio between the positively charged components and the negatively charged components in the mixture is determined by appropriately adjusting the concentrations of each solution or by adjusting the volume of solution added. In another embodiment, the two solutions are mixed under continuous flow conditions using mixing apparatus such as static mixer. In this embodiment, two or more solutions are introduced into a static mixer at rates and pressures giving a ratio of the solutions, where the streams of solutions get mixed within the static mixer. Arrangements are possible for mixers to be arranged in parallel or in series.

In one embodiment, the present invention provides for formulations for siRNA oligonucleotides that comprise tissue-targetable delivery with three properties. These are nucleic acid binding into a core that can release the siRNA into the cytoplasm, protection from non-specific interactions, and tissue targeting that provides cell uptake. The invention provides for compositions and methods that use modular conjugates of three materials to combine and assemble the multiple properties required. They can be designed and synthesized to incorporate various properties and then mixed with the siRNA payload to form the nanoparticles. One embodiment comprises a modular polymer conjugate targeting neovasculature by coupling a peptide ligand specific for those cells to one end of a protective polymer, coupled at its other end to a cationic carrier for nucleic acids. This polymer conjugate has three functional domains, sometimes referred to as a tri-functional polymer (TFP). The modular design of this conjugate allows replacement and optimization of each component separately. An alternative approach has been to attach surface coatings onto pre-formed nanoparticles. Adsorption of a steric polymer coating onto polymers is self-limiting; once a steric layer begins to form it will impede further addition of polymer. The compositions and methods of the invention permit an efficient method for optimization of each of the three functions, largely independent of the other two functions.

### Protective Steric Coating for Nucleic Acid Nanoparticles

Even liposomes with an external lipid bilayer resembling the outer cellular membrane are rapidly recognized and cleared from blood. Nanotechnology offers a broad range of synthetic polymer chemistry. Hydrophilic polymers, such as PEG and polyacetals and polyoxazolines, have proven effective to form a “steric” protective layer on the surface of colloidal drug delivery systems whether liposomes, polymer or electrostatic nanoparticles, reducing immune clearance from blood. The use of this steric PEG layer was first developed and most extensively studied with sterically stabilized liposomes. The present invention provides for alternative approaches, such as chemical reduction of surface charge, in addition to a steric polymer coating.

The steric barrier and biological consequences appear to derive from physical, not chemical, properties. Several other hydrophilic polymers have been reported as alternatives to PEG. Physical studies on sterically stabilized liposomes have provided a strong mechanistic underpinning for physical behavior of the polymer layer and can be used to achieve similar coatings on other types of particles. However, while physical studies have shown formation of a similar polymer layer on the surface of polymer complexes with nucleic acids, and achievement of similar biological properties, we lack sufficient information today to use of the physical properties to accurately predict protection from immune clearance from blood. Liposome studies indicate that physical properties with the greatest impact on biological activity can be obtained by synthesis of a matrix of conjugates varying the size of the two polymers and the grafting density. Note that while the surface steric layer function is due to physical properties, the optimal conjugation chemistry still depends on the specific chemical nature of the steric polymer and the carrier to which it is coupled.

Methods for formation of the nanoparticles with the surface steric polymer layer are also an important parameter. One embodiment the steric polymer is coupled to the carrier polymer to give a conjugate that self-assembles with the nucleic acid forming a nanoparticle with the steric polymer surface layer. In another embodiment surface coatings are attached onto pre-formed nanoparticles. In self-assembly, formation of the surface steric layer depends on interactions of the carrier polymer with the payload, not on penetration through a forming steric layer to react with the particle surface. In this case, effects of the steric polymer on the ability of the carrier polymer to bind the nucleic acid payload may have adverse effects on particle formation, and thus the surface steric layer. If this occurs, the grafting density of the steric polymer on the carrier will have exceeded its maximum, or the structural nature of the grafting is not adequate.

### Surface Exposed Ligands and Moieties Targeting Specific Tissues

The ability of the nanoparticle to selectively reach the interior of the target cells resides in its ability to induce a specific receptor mediated uptake. This is provided in the present invention by exposed ligands or targeting moieties which provide the binding specificity. Many types of ligands exist for targeting colloidal delivery systems. One such method involves coupling antibodies to the surface of liposomes, usually referred to as immunoliposomes. One important parameter that has emerged is the impact of ligand density. Antibodies tend to meet many requirements for use as the ligand, including good binding selectivity and nearly routine preparation for nearly any receptor and broad applicability of protein coupling methods regardless of nanoparticle type. Monoclonal antibodies even show signs of being able to cross the blood-brain-barrier. Other proteins that are natural ligands and receptors also have been considered for targeting nanoparticles, such as transferrin or transferrin receptor. In one embodiment of the invention, the targeting ligand or moiety may be a sugar or a sugar analogue.

A preferred class of ligands are small molecular weight compounds with strong selective binding affinity for internalizing receptors. Studies have evaluated natural metabolites including vitamins such as folate and thiamine, polysaccharides such as wheat germ agglutinin or sialyl Lewis^{X} for e-selectin, and peptide binding domains such as RGD for integrins. Peptides offer a versatile class of ligand, since phage display libraries can be used to screen for natural or unnatural sequences, even with in vivo panning methods. Such phage display methods can permit retention of an unpaired Cys residue at one end for ease of coupling regardless of sequence. Use of an RGD peptide for targeted delivery of nanoparticles to neovasculature can be very effective to meet the major requirements for effective ligands: specific chemistry that doesn't interfere with ligand binding or induce immune clearance yet enables selective receptor mediated uptake at the target cells.

The compositions and methods of the present invention provide for administration of siRNA with nucleic acid delivery vehicles comprising polymers, polymer conjugates, lipids, micelles, self-assembly colloids, nanoparticles, sterically stablized nanoparticles, or ligand-directed nanoparticles. Targeted synthetic vectors of the type described in WO 01/49324 and U.S. Patent Application Publication No. 2003/0166601, which are hereby incorporated by reference in its entirety, may be used for systemic delivery of RNAi-inducing nucleic acid molecules of the present invention. In one embodiment, a PEI-PEG-RGD (polyethyleneimine-polyethylene glycol-argine-glycine-aspartic acid) synthetic vector can be prepared and used, for example as in Examples 53 and 56 of WO 01/49324 and U.S. Patent Application Publication No. 2003/0166601. This vector was used to deliver RNAi systemically via intravenous injection. Other targeted synthetic vector molecules known in the art may also be used. For example, the vector may have an inner shell made up of a core complex comprising the RNAi and at least one complex forming reagent. The vector also may contain a fusogenic moiety, which may comprise a shell that is anchored to the core complex, or may be incorporated directly into the core complex. The vector may further have an outer shell moiety that stabilizes the vector and reduces nonspecific binding to proteins and cells. The outer shell moiety may comprise a hydrophilic polymer, and/or may be anchored to the fusogenic moiety. The outer shell moiety may be anchored to the core complex. The vector may contain a targeting moiety that enhances binding of the vector to a target tissue and cell population. Suitable targeting moieties are known in the art and are described in detail in WO 01/49324 and U.S. Patent Application Publication No. 2003/0166601.

One embodiment of the present invention provides compositions and methods for RGD-mediated ligand-directed nanoparticle preparations of anti-VEGF pathway siRNA short double stranded RNA molecules. In one method for the manufacture of RGD-mediated tissue targeted nanoparticles containing siRNA, the targeting ligand, an RGD containing peptide (ACRGDMFGCA (SEQ ID NO: 12)) is conjugated to a steric polymer such as polyethylene glycol, or other polymers with similar properties (see WO 06/110813, incorporated herein by reference in its entirety). This ligand-steric polymer conjugate is further conjugated to a polycation such as polyethyleneimine or other effective material such as a histidine-lysine copolymer. The conjugation can be by covalent or non-covalent bonds and the covalent bonds can be non-cleavable or they can be cleavable such as by hydrolysis or by reducing agents. A solution comprising the polymer conjugate, or comprising a mixture of a polymer conjugate with other polymer, lipid, or micelle such as materials comprising a ligand or a steric polymer or fusogen, is mixed with a solution comprising the nucleic acid, in one embodiment an siRNA targeted against specific genes of interest, in desirable ratios to obtain nanoparticles that contain siRNA.

### Combined Formulation and Electric Field

For certain applications, siRNA may be administered with or without application of an electric field. This can be used, for example, to deliver the siRNA molecules of the invention via direct injections into, for example, tumor tissue and directly into or nearby an angiogenic tissue or a tissue with undesirable neovasculature. The siRNA may be in a suitable pharmaceutical carrier such as, for example, a saline solution or a buffered saline solution.

The present invention, thus generally described, will be understood more readily by reference to the following examples, which are provided by way of illustration and are not intended to be limiting of the present invention.

### Example 1

### Candidate siRNA Molecules for Reducing Human VEGFR1 Expression

Human VEGFR1 siRNA molecules were designed using a tested algorithm and using the publicly available sequences for human VEGFR1 mRNA (GenBank Accession No. AF063657; FIGS. 7A and 7B; SEQ ID NO: 197), human soluble VEGFR1 mRNA (GenBank Accession No. U01134; FIG. 8; SEQ ID NO: 198), and mouse VEGFR1 mRNA (GenBank Accession No. NM_{—}010228.2; FIGS. 9A and 9B; SEQ ID NO: 199).

Exemplary siRNAs targeting both soluble and membrane-bound hVEGFR1 are shown in Table 1 above. Exemplary siRNAs targeting membrane-bound hVEGFR1 but not soluble hVEGFR1 are shown in Table 2 above. Exemplary siRNAs targeting both human and mouse VEGFR1 are shown in Table 3 above.

### Example 2

### siRNA Molecules Inhibit Full-Length Human VEGFR1 Protein Expression without Affecting Soluble Human VEGFR1 Protein Expression

A total of 48 blunt-ended 25-mer siRNAs targeting human VEGFR1 were tested in HUVEC cells for their potency in knockdown of human VEGFR1 (“hVEGFR1”) expression in the transfected cells. The 48 hVEGFR1-siRNAs were chosen from the lists of hVEGFR1-siRNA in Tables 1-3 and 7-11, synthesized by Qiagen Inc. (Germantown, Md.), and subjected to potency screening in HUVEC cells. Among the 48 siRNAs, hVEGFR1-siRNAs #1-19 (Table 4) target both mRNAs coding for soluble (truncated) hVEGFR1 and full-length membrane-bound hVEGFR1. In contrast, hVEGFR1-siRNAs #20-48 (Table 5) target only the full-length hVEGFR1 mRNA.

TABLE 4

List of siRNAs targeting both the mRNA encoding soluble hVEGFR1 and
full-length hVEGFR1 mRNA

| | | | | |
|---|---|---|---|---|
| | | | | Sense |
| | | | | strand |
| | | | | SEQ ID |
| No. | Start | siRNA sequence (sense strand/antisense strand) | GC % | NO |

| | | | | |
|---|---|---|---|---|
| hVEGFR- | 173 | 5′-CCCAUAAAUGGUCUUUGCCUGAAAU-3′ | 40.0 | 129 |
| 25-1 | | 3′-GGGUAUUUACCAGAAACGGACUUUA-5′ | | |
| hVEGFR- | 222 | 5′-GAGCAUAACUAAAUCUGCCUGUGGA-3′ | 44.0 | 130 |
| 25-2 | | 3′-CUCGUAUUGAUUUAGACGGACACCU-5′ | | |
| hVEGFR- | 252 | 5′-UGGCAAACAAUUCUGCAGUACUUUA-3′ | 36.0 | 131 |
| 25-3 | | 3′-ACCGUUUGUUAAGACGUCAUGAAAU-5′ | | |
| hVEGFR- | 351 | 5′-GAAGGAAACAGAAUCUGCAAUCUAU-3′ | 36.0 | 136 |
| 25-4 | | 3′-CUUCCUUUGUCUUAGACGUUAGAUA-5′ | | |
| hVEGFR- | 392 | 5′-CAGGUAGACCUUUCGUAGAGAUGUA-3′ | 44.0 | 137 |
| 25-5 | | 3′-GUCCAUCUGGAAAGCAUCUCUACAU-5′ | | |
| hVEGFR- | 626 | 5′-CAACAGUCAAUGGGCAUUUGUAUAA-3′ | 36.0 | 142 |
| 25-6 | | 3′-CGUUGUCAGUUACCCGUAAACAUAU-5′ | | |
| hVEGFR- | 720 | 5′-CAAAUUACUUAGAGGCCAUACUCUU-3′ | 36.0 | 145 |
| 25-7 | | 3′-GUUUAAUGAAUCUCCGGUAUGAGAA-5′ | | |
| hVEGFR- | 733 | 5′-GGCCAUACUCUUGUCCUCAAUUGUA-3′ | 44.0 | 146 |
| 25-8 | | 3′-CCGGUAUGAGAACAGGAGUUAACAU-5′ | | |
| hVEGFR- | 744 | 5′-UGUCCUCAAUUGUACUGCUACCACU-3′ | 44.0 | 147 |
| 25-9 | | 3′-ACAGGAGUUAACAUGACGAUGGUGA-5′ | | |
| hVEGFR- | 764 | 5′-CCACUCCCUUGAACACGAGAGUUCA-3′ | 52.0 | 148 |
| 25-10 | | 3′-GGUGAGGGAACUUGUGCUCUCAAGU-5′ | | |
| hVEGFR- | 1050 | 5′-GCGGUCUUACCGGCUCUCUAUGAAA-3′ | 52.0 | 149 |
| 25-11 | | 3′-CGCCAGAAUGGCCGAGAGAUACUUU-5′ | | |
| hVEGFR- | 1086 | 5′-UCCCUCGCCGGAAGUUGUAUGGUUA-3′ | 52.0 | 150 |
| 25-12 | | 3′-AGGGAGCGGCCUUCAACAUACCAAU-5′ | | |
| hVEGFR- | 1087 | 5′-CCCUCGCCGGAAGUUGUAUGGUUAA-3′ | 52.0 | 6 |
| 25-13 | | 3′-GGGAGCGGCCUUCAACAUACCAAUU-5′ | | |
| hVEGFR- | 1125 | 5′-UGCGACUGAGAAAUCUGCUCGCUAU-3′ | 48.0 | 151 |
| 25-14 | | 3′-ACGCUGACUCUUUAGACGAGCGAUA-5′ | | |
| hVEGFR- | 1147 | 5′-UAUUUGACUCGUGGCUACUCGUUAA-3′ | 40.0 | 152 |
| 25-15 | | 3′-AUAAACUGAGCACCGAUGAGCAAUU-5′ | | |
| hVEGFR- | 1201 | 5′-GGGAAUUAUACAAUCUUGCUGAGCA-3′ | 40.0 | 154 |
| 25-16 | | 3′-CCCUUAAUAUGUUAGAACGACUCGU-5′ | | |
| hVEGFR- | 1344 | 5′-CAGACAAAUCCUGACUUGUACCGCA-3′ | 48.0 | 157 |
| 25-17 | | 3′-GUCUGUUUAGGACUGAACAUGGCGU-5′ | | |
| hVEGFR- | 1576 | 5′-GACUCUAGAAUUUCUGGAAUCUACA-3′ | 36.0 | 158 |
| 25-18 | | 3′-CUGAGAUCUUAAAGACCUUAGAUGU-5′ | | |
| hVEGFR- | 1847 | 5′-CCAUCACUCUUAAUCUUACCAUCAU-3′ | 36.0 | 164 |
| 25-19 | | 3′-GGUAGUGAGAAUUAGAAUGGUAGUA-5′ | | |

TABLE 5
List of siRNA targeting only full-length hVEGFR1 mRNA

| | | | | |
|---|---|---|---|---|
| | | | | Sense |
| | | | | strand |
| | | | | SEQ ID |
| No. | Start | siRNA sequence (sense strand/antisense strand) | GC % | NO |

| | | | | |
|---|---|---|---|---|
| hVEGFR- | 2030 | 5′-CCACCACUUUAGACUGUCAUGCUAA-3′ | 44.0 | 83 |
| 25-20 | | 3′-GGUGGUGAAAUCUGACAGUACGAUU-5′ | | |
| hVEGFR- | 2141 | 5′-GCACGCUGUUUAUUGAAAGAGUCAC-3′ | 44.0 | 196 |
| 25-21 | | 3′-CGUGCGACAAAUAACUUUCUCAGUG-5′ | | |
| hVEGFR- | 2142 | 5′-CACGCUGUUUAUUGAAAGAGUCACA-3′ | 36.0 | 100 |
| 25-22 | | 3′-GUGCGACAAAUAACUUUCUCAGUGU-5′ | | |
| hVEGFR- | 2144 | 5′-CGCUGUUUAUUGAAAGAGUCACAGA-3′ | 36.0 | 50 |
| 25-23 | | 3′-GCGACAAAUAACUUUCUCAGUGUCU-5′ | | |
| hVEGFR- | 2145 | 4′-GCUGUUUAUUGAAAGAGUCACAGAA-3′ | 36.0 | 49 |
| 25-24 | | 3′-CGACAAAUAACUUUCUCAGUGUCUU-5′ | | |
| hVEGFR- | 2255 | 5′-CGGACAAGUCUAAUCUGGAGCUGAU-3′ | 48.0 | 84 |
| 25-25 | | 3′-GCCUGUUCAGAUUAGACCUCGACUA-5′ | | |
| hVEGFR- | 2305 | 5′-GCGACUCUCUUCUGGCUCCUAUUAA-3′ | 48.0 | 173 |
| 25-26 | | 3′-CGCUGAGAGAAGACCGAGGAUAAUU-5′ | | |
| hVEGFR- | 2645 | 5′-UGACCCACAUUGGCCACCAUCUGAA-3′ | 52.0 | 85 |
| 25-27 | | 3′-ACUGGGUGUAACCGGUGGUAGACUU-5′ | | |
| hVEGFR- | 2705 | 5′-GAGGGCCUCUGAUGGUGAUUGUUGA-3′ | 52.0 | 86 |
| 25-28 | | 3′-CUCCCGGAGACUACCACUAACAACU-5′ | | |
| hVEGFR- | 2753 | 5′-CCAACUACCUCAAGAGCAAACGUGA-3′ | 48.0 | 24 |
| 25-29 | | 3′-UGGAGUUCUCGUUUGCACUGAAUAA-5′ | | |
| hVEGFR- | 2757 | 5′-CUACCUCAAGAGCAAACGUGACUUA-3′ | 44.0 | 25 |
| 25-30 | | 3′-GAUGGAGUUCUCGUUUGCACUGAAU-5′ | | |
| hVEGFR- | 2759 | 5′-ACCUCAAGAGCAAACGUGACUUAUU-3′ | 40.0 | 51 |
| 25-31 | | 3′-UGGAGUUCUCGUUUGCACUGAAUAA-5′ | | |
| hVEGFR- | 2760 | 5′-CCUCAAGAGCAAACGUGACUUAUUU-3′ | 40.0 | 43 |
| 25-32 | | 3′-GGAGUUCUCGUUUGCACUGAAUAAA-5′ | | |
| hVEGFR- | 2900 | 5′-CGAGCUCCGGCUUUCAGGAAGAUAA-3′ | 52.0 | 87 |
| 25-33 | | 3′-GCUCGAGGCCGAAAGUCCUUCUAUU-5′ | | |
| hVEGFR- | 2901 | 5′-GAGCUCCGGCUUUCAGGAAGAUAAA-3′ | 48.0 | 180 |
| 25-34 | | 3′-CUCGAGGCCGAAAGUCCUUCUAUUU-5′ | | |
| hVEGFR- | 3027 | 5′-CAUGGAGUUCCUGUCUUCCAGAAAG-3′ | 48.0 | 181 |
| 25-35 | | 3′-GUACCUCAAGGACAGAAGGUCUUUC-5′ | | |
| hVEGFR- | 3031 | 5′-GAGUUCCUGUCUUCCAGAAAGUGCA-3′ | 48.0 | 182 |
| 25-36 | | 3′-CUCAAGGACAGAAGGUCUUUCACGU-5′ | | |
| hVEGFR- | 3347 | 5′-GCAUGAGGAUGAGAGCUCCUGAGUA-3′ | 52.0 | 183 |
| 25-37 | | 3′-CGUACUCCUACUCUCGAGGACUCAU-5′ | | |
| hVEGFR- | 3357 | 5′-GAGAGCUCCUGAGUACUCUACUCCU-3′ | 52.0 | 184 |
| 25-38 | | 3′-CUCUCGAGGACUCAUGAGAUGAGGA-5′ | | |
| hVEGFR- | 3458 | 5′-AACUAGGUGAUUUGCUUCAAGCAAA-3′ | 36.0 | 186 |
| 25-39 | | 3′-UUGAUCCACUAAACGAAGUUCGUUU-5′ | | |
| hVEGFR- | 3462 | 5′-AGGUGAUUUGCUUCAAGCAAAUGUA-3′ | 36.0 | 187 |
| 25-40 | | 3′-UCCACUAAACGAAGUUCGUUUACAU-5′ | | |
| hVEGFR- | 3512 | 5′-CAAUCAAUGCCAUACUGACAGGAAA-3′ | 40.0 | 88 |
| 25-41 | | 3′-GUUAGUUACGGUAUGACUGUCCUUU-5′ | | |
| hVEGFR- | 3527 | 5′-UGACAGGAAAUAGUGGGUUUACAUA-3′ | 36.0 | 188 |
| 25-42 | | 3′-ACUGUCCUUUAUCACCCAAAUGUAU-5′ | | |
| hVEGFR- | 3586 | 5′-GAAAGUAUUUCAGCUCCGAAGUUUA-3′ | 36.0 | 89 |
| 25-43 | | 3′-CUUUCAUAAAGUCGAGGCUUCAAAU-5′ | | |
| hVEGFR- | 3660 | 5′-GAGCCUGGAAAGAAUCAAAACCUUU-3′ | 40.0 | 104 |
| 25-44 | | 3′-CUGGGACCUUUCUUAGUUUUGGAAA-5′ | | |
| hVEGFR- | 3662 | 5′-GCCUGGAAAGAAUCAAAACCUUUGA-3′ | 40.0 | 105 |
| 25-45 | | 3′-CGGACCUUUCUUAGUUUUGGAAACU-5′ | | |
| hVEGFR- | 3810 | 5′-GAUUGACUUGAGAGUAACCAGUAAA-3′ | 36.0 | 192 |
| 25-46 | | 3′-CUAACUGAACUCUCAUUGGUCAUUU-5′ | | |
| hVEGFR- | 3974 | 5′-CAGACUACAACUCGGUGGUCCUGUA-3′ | 52.0 | 193 |
| 25-47 | | 3′-GUCUGAUGUUGAGCCACCAGGACAU-5′ | | |
| hVEGFR- | 3976 | 5′-GACUACAACUCGGUGGUCCUGUACU-3′ | 52.0 | 194 |
| 25-48 | | 3′-CUGAUGUUGAGCCACCAGGACAUGA-5′ | | |

HUVEC cells (Cambrex, Walkersville, Md., USA) were cultured in EGM-2 medium (Cambrex) containing 2% FBS at 37° C. in an incubator with 5% CO₂. HUVECs at passage three to five were used for siRNA transfection. A reverse or forward siRNA-transfection procedure was performed with Lipofectamine RNAiMax Reagent (Invitrogen) in HUVEC cells using a concentration of siRNA of 10-20 nM following manufacturer's protocol. siRNA transfections were performed in 48-well plates (duplicates for each siRNA sequence) for ELISA assay or in 96-well plate for RealTime-PCR assay. AllStars Negative Control siRNA from Qiagen or Luc-siRNA were used as the negative control for hVEGFR1 siRNA potency screening (see Table 12).

For detection of siRNA mediated knockdown of hVEGFR1 at protein levels, cell culture supernatants and cell lysates of transfected HUVEC cells were collected at 48 h post-transfection for measurement of soluble hVEGFR1 present in cell culture supernatants and total hVEGFR1 present in cell lysates using hVEGFR1 ELISA assay (R&D system). A significant knockdown of soluble hVEGFR1 protein in the culture supernatant of the transfected HUVEC cells was observed when HUVEC cells were transfected with siRNAs #1-19 that target both mRNAs coding for soluble and membrane-bound full-length hVEGFR1 (FIG. 1A), but not in the cells transfected with siRNAs #20-48 that target only the full-length hVEGFR1 mRNA coding for the membrane-bound hVEGFR1 (FIG. 2A). There is a significant knockdown of total hVEGFR1 protein (including both soluble hVEGFR1 and membrane-bound hVEGFR1) in HUVEC cells transfected with siRNAs #1-19 (FIG. 1B), in contrast to some levels of enhancement of total hVEGFR1 protein by transfection of siRNAs #20-48 (FIG. 2B).

The inhibition of hVEGFR1 protein expression in both supernatant and cell lysate by the 48 tested siRNAs is summarized in Table 6. The data in Table 6 are graphed in FIGS. 3 and 4.

TABLE 6
Summary of individual siRNA's
inhibition of hVEGFR1 expression

| | | % inhibition |
|---|---|---|
| siRNA | ELISA sample | (to control) |
| hVEGFR1-25-1 | supernatant | 56 ± 0.43 |
| | cell lysate | 42.3 ± 2.1 |
| hVEGFR1-25-2 | supernatant | 76.6 ± 1.2 |
| | cell lysate | 69.2 ± 0.66 |
| hVEGFR1-25-3 | supernatant | 85.9 ± 0.97 |
| | cell lysate | 78.8 ± 1.02 |
| hVEGFR1-25-4 | supernatant | 88.5 ± 0.41 |
| | cell lysate | 88 ± 0.12 |
| hVEGFR1-25-5 | supernatant | 84.9 ± 0.53 |
| | cell lysate | 82.7 ± 1.11 |
| hVEGFR1-25-6 | supernatant | 17.5 ± 18.9 |
| | cell lysate | 4.78 ± 7.68 |
| hVEGFR1-25-7 | supernatant | 49.6 ± 3.44 |
| | cell lysate | 42.7 ± 4.2 |
| hVEGFR1-25-8 | supernatant | 84.4 ± 0.65 |
| | cell lysate | 79.5 ± 0.6 |
| hVEGFR1-25-9 | supernatant | 44.4 ± 0.16 |
| | cell lysate | 33.3 ± 0.57 |
| hVEGFR1-25-10 | supernatant | 67.8 ± 2.41 |
| | cell lysate | 53.7 ± 1.0 |
| hVEGFR1-25-11 | supernatant | 48.2 ± 0.36 |
| | cell lysate | 42.4 ± 3.5 |
| hVEGFR1-25-12 | supernatant | 55.9 ± 1.46 |
| | cell lysate | 54.7 ± 0.1 |
| hVEGFR1-25-13 | supernatant | 55.9 ± 0.27 |
| | cell lysate | 46.7 ± 5.84 |
| hVEGFR1-25-14 | supernatant | 63.6 ± 1.14 |
| | cell lysate | 52.9 ± 3.39 |
| hVEGFR1-25-15 | supernatant | 72.9 ± 1.81 |
| | cell lysate | 62.6 ± 3.11 |
| hVEGFR1-25-16 | supernatant | 64.4 ± 2.94 |
| | cell lysate | 57.5 ± 2.71 |
| hVEGFR1-25-17 | supernatant | 87.5 ± 0.71 |
| | cell lysate | 75.7 ± 1.28 |
| hVEGFR1-25-18 | supernatant | 68.3 ± 4.6 |
| | cell lysate | 67.4 ± 5.36 |
| hVEGFR1-25-19 | supernatant | 80 ± 1.2 |
| | cell lysate | 71.3 ± 2.6 |
| hVEGFR1-25-20 | supernatant | 37.3 ± 3.66 |
| | cell lysate | 25.4 ± 4.71 |
| hVEGFR1-25-21 | supernatant | — |
| | cell lysate | — |
| hVEGFR1-25-22 | supernatant | — |
| | cell lysate | — |
| hVEGFR1-25-23 | supernatant | 4.2 ± 9.14 |
| | cell lysate | 18.1 ± 0.55 |
| hVEGFR1-25-24 | supernatant | — |
| | cell lysate | 1.03 |
| hVEGFR1-25-25 | supernatant | — |
| | cell lysate | 6.5 ± 1.3 |
| hVEGFR1-25-26 | supernatant | — |
| | cell lysate | 22.2 ± 1.43 |
| hVEGFR1-25-27 | supernatant | 3.88 ± 4.68 |
| | cell lysate | — |
| hVEGFR1-25-28 | supernatant | — |
| | cell lysate | — |
| hVEGFR1-25-29 | supernatant | — |
| | cell lysate | — |
| hVEGFR1-25-30 | supernatant | — |
| | cell lysate | 12.8 ± 4.0 |
| hVEGFR1-25-31 | supernatant | 30.4 ± 2.06 |
| | cell lysate | — |
| hVEGFR1-25-32 | supernatant | — |
| | cell lysate | — |
| hVEGFR1-25-33 | supernatant | 4.3 ± 1.85 |
| | cell lysate | — |
| hVEGFR1-25-34 | supernatant | 7.03 ± 7.6 |
| | cell lysate | — |
| hVEGFR1-25-35 | supernatant | 21.8 ± 0.48 |
| | cell lysate | 9.8 ± 0.91 |
| hVEGFR1-25-36 | supernatant | 12.9 ± 2.89 |
| | cell lysate | — |
| hVEGFR1-25-37 | supernatant | 14.5 ± 3.4 |
| | cell lysate | — |
| hVEGFR1-25-38 | supernatant | 6.4 ± 0.44 |
| | cell lysate | — |
| hVEGFR1-25-39 | supernatant | — |
| | cell lysate | — |
| hVEGFR1-25-40 | supernatant | — |
| | cell lysate | 6.8 ± 0.7 |
| hVEGFR1-25-41 | supernatant | 15.0 ± 0.89 |
| | cell lysate | — |
| hVEGFR1-25-42 | supernatant | — |
| | cell lysate | — |
| hVEGFR1-25-43 | supernatant | — |
| | cell lysate | — |
| hVEGFR1-25-44 | supernatant | — |
| | cell lysate | — |
| hVEGFR1-25-45 | supernatant | — |
| | cell lysate | — |
| hVEGFR1-25-46 | supernatant | — |
| | cell lysate | — |
| hVEGFR1-25-47 | supernatant | — |
| | cell lysate | — |
| hVEGFR1-25-48 | supernatant | — |
| | cell lysate | — |

Note:
“—” = no inhibition.

Because the ELISA assay cannot distinguish soluble hVEGFR1 from full-length membrane-bound hVEGFR1 when total hVEGFR1 protein levels are measured in the cell lysates, a quantitative RealTime-PCR (“QRT-PCR”) assay was used to measure the knockdown of full-length hVEGFR1 specifically.

For detection of siRNA mediated knockdown of hVEGFR1 at mRNA levels, HUVEC cells were transfected with 10 nM siRNA and the cells were collected at 48 hour post-transfection for measurement of the relative levels of hVEGFR1 mRNAs using QRT-PCR assays with either a full-length hVEGFR1 mRNA specific gene expression assay (Hs_{—}0176573_ml, ABI) or a gene expression assay for both mRNAs coding for soluble and the membrane-bound hVEGFR1 (Hs_{—}01052936_ml, ABI). The cells were lysed using “Cell to Signal Kit” for QRT-PCR assay. The samples were stored at −80° C. A significant knockdown of total hVEGFR1 mRNAs was observed only in HUVEC cells transfected with hVEGFR1-siRNAs #1-19 (FIG. 5, gray bars), but not in HUVEC cells transfected with hVEGFR1-siRNAs #20-48 (FIG. 6, gray bars), which is consistent with protein knockdown data (FIGS. 1A, 1B, 2A, 2B, 3 and 4). However, a significant knockdown of the mRNA coding for the full-length membrane-bound hVEGFR1 was observed in HUVEC cells transfected with all of the hVEGFR1-siRNAs (FIGS. 5 and 6, black bars). This is a clear indication that hVEGFR1-siRNAs #20-48 specifically knock down only the full-length hVEGFR1 mRNA, but not the soluble hVEGFR1 mRNA.

In conclusion, through conducting in vitro siRNA screening in HUVEC cells, we have demonstrated that several of our siRNA candidates are very potent for inhibition of hVEGFR1 gene expression at both protein and mRNA levels. In addition, we also have demonstrated that these siRNAs reduce only the membrane-bound full-length hVEGFR1 without affecting the soluble hVEGFR1. We have surprisingly discovered that several full-length hVEGFR1-specific siRNAs increased the level of soluble hVEGFR1 (see e.g. FIGS. 2A and 6 for hVEGFR1-siRNAs # 21-25, 27-29, 31, 38, 39, and 41-48).

TABLE 7
siRNA sequences targeting VEGF pathway genes

| | | | |
|---|---|---|---|
| | Start | | |
| Name of siRNA | site | Target sequence (sense strand) | GC % |

| | | | |
|---|---|---|---|
| hVEGF-21a | 162 | aaucgagacccugguggacau (SEQ ID NO: 44) | 58 |
| | | aatcgagaccctggtggacat (SEQ ID NO: 40) | |
| hVEGF-21b | 338 | aaggccagcacauaggagaga (SEQ ID NO: 45) | 52 |
| hVEGF-siRNA-25-1 | 158 | auccaaucgagacccugguggacau (SEQ ID NO: 46) | 52 |
| hVEGF-siRNA-25-2 | 159 | uccaaucgagacccugguggacauc (SEQ ID NO: 47) | 56 |
| hVEGF-siRNA-25-3 | 160 | ccaaucgagacccugguggacaucu (SEQ ID NO: 128) | 56 |
| hVEGF-siRNA-25-4 | 161 | caaucgagacccugguggacaucuu (SEQ ID NO: 41) | 52 |
| hVEGF-siRNA-25-5 | 162 | aaucgagacccugguggacaucuuc (SEQ ID NO: 42) | 52 |
| hVEGF-siRNA-25-a | 196 | ccugaugagaucgaguacaucuuca (SEQ ID NO: 1) | 44 |
| hVEGF-siRNA-25-b | 353 | gagagaugagcuuccuacagcacaa (SEQ ID NO: 2) | 48 |
| hVEGF-siRNA-25-c | 373 | cacaacaaaugugaaugcagaccaa (SEQ ID NO: 3) | 40 |
| mhVEGF-siRNA-25-1 | | caagauccgcagacguguaaauguu (SEQ ID NO: 20) | 44 |
| hmVEGF-siRNA-25-4 | | ccgcagacguguaaauguuccugca (SEQ ID NO: 14) | 52 |
| hmVEGF-siRNA-25-2 | | gcagacguguaaauguuccugcaaa (SEQ ID NO: 15) | 44 |
| hmVEGF-siRNA-25-6 | | gcaaggcgaggcagcuugaguuaaa (SEQ ID NO: 13) | 52 |
| mhVEGF-siRNA-25-2 | | gcagcuugaguuaaacgaacguacu (SEQ ID NO: 21) | 44 |
| hmVEGF-siRNA-25-3 | | cagcuugaguuaaacgaacguacuu (SEQ ID NO: 48) | 40 |
| mhVEGF-siRNA-25-4 | | ccaugccaaguggucccaggcugca (SEQ ID NO: 22) | 63 |
| mhVEGF-siRNA-25-6 | | cccugguggacaucuuccaggagua (SEQ ID NO: 23) | 56 |
| hVEGFR1-siRNA-25-a | 865 | gccaacauauucuacaguguucuua (SEQ ID NO: 5) | 36 |
| hVEGFR1-siRNA-25-b | 1087 | cccucgccggaaguuguaugguuaa (SEQ ID NO: 6) | 52 |
| hVEGFR1-SiRNA-25-c | 2760 | ccucaagagcaaacgugacuuauuu (SEQ ID NO: 43) | 40 |
| hmVEGFR1-siRNA-25-1 | 2145 | gcuguuuauugaaagagucacagaa (SEQ ID NO: 49) | 36 |
| hmVEGFR1-siRNA-25-2 | 2144 | cgcuguuuauugaaagagucacaga (SEQ ID NO: 50) | 40 |
| hmVEGFR1-siRNA-25-3 | 2760 | ccucaagagcaaacgugacuuauuu (SEQ ID NO: 43) | 40 |
| hmVEGFR1-siRNA-25-4 | 2759 | accucaagagcaaacgugacuuauu (SEQ ID NO: 51) | 40 |
| mhVEGFR1-siRNA-25-5 | | cuaccucaagagcaaacgugacuua (SEQ ID NO: 25) | 44 |
| hVEGFR2-siRNA-25-a | 246 | ccucuucuguaagacacucacaauu (SEQ ID NO: 8) | 40 |
| hVEGFR2-siRNA-25-b | 1109 | cccuugaguccaaucacacaauuaa (SEQ ID NO: 9) | 40 |
| hVEGFR2-siRNA-25-c | 2538 | ccaagugauugaagcagaugccuuu (SEQ ID NO: 10) | 44 |
| hmVEGFR2-siRNA-25-1 | 3510 | caucucaucuguuacagcuuccaag (SEQ ID NO: 52) | 48 |
| hmVEGFR2-siRNA-25-2 | 3555 | cauggaagaggauucuggacucucu (SEQ ID NO: 53) | 48 |
| hmVEGFR2-siRNA-25-3 | 3531 | caaguggcuaagggcauggaguucu (SEQ ID NO: 54) | |
| hmVEGFR2-siRNA-25-5 | 2384 | gggaacugaagacaggcuacuuguc (SEQ ID NO: 55) | |
| mhVEGFR2-siRNA-25-6 | | gacuuccugaccuuggagcaucuca (SEQ ID NO: 29) | |
| hVEGFR2-siRNA-21-6 | 178 | gacuggcuuuggcccaauaauca (SEQ ID NO: 56) | 47 |
| mVEGF-siRNA-25-1 | 587 | cccgacgagauagaguacaucuuca (SEQ ID NO: 57) | 48 |
| mVEGF-siRNA-25-2 | 588 | ccgacgagauagaguacaucuucaa (SEQ ID NO: 58) | 44 |
| hmVEGF-siRNA-25-1 | 857 | caagauccgcagacguguaaauguu (SEQ ID NO: 20) | 44 |
| hmVEGF-siRNA-25-4 | 863 | ccgcagacguguaaauguuccugca (SEQ ID NO: 14) | 52 |
| hmVEGF-siRNA-25-2 | 865 | gcagacguguaaauguuccugcaaa (SEQ ID NO: 15) | 44 |
| hmVEGF-siRNA-25-6 | 906 | gcaaggcgaggcagcuugaguuaaa (SEQ ID NO: 13) | 52 |
| hmVEGF-siRNA-25-2 | 916 | gcagcuugaguuaaacgaacguacu (SEQ ID NO: 21) | 44 |
| hmVEGF-siRNA-25-3 | 917 | cagcuugaguuaaacgaacguacuu (SEQ ID NO: 48) | 40 |
| mVEGFR1-21a | | aaguuaaaagugccugaacug (SEQ ID NO: 59) | |
| mVEGFR1-21b | | aagcaggccagacucucuuuc (SEQ ID NO: 60) | |
| mVEGFR1-siRNA-25-a | 612 | gcggaaucuucaaucuacauauuug (SEQ ID NO: 16) | 36 |
| mVEGFR1-siRNA-25-b | 811 | gggacaguaggagaggcuuuauaau (SEQ ID NO: 39) | 44 |
| mVEGFR1-siRNA-25-c | 2899 | ugacccacaucggccaucaucugaa (SEQ ID NO: 61) | 52 |
| mVEGFR2-21a | | aagcucagcacacagaaagac (SEQ ID NO: 62) | |
| | | aagctcagcacacagaaagac (SEQ ID NO: 37) | |
| mVEGFR2-21b | | aaugcggcgguggugacagua (SEQ ID NO: 63) | |
| | | aatgcggcggtggtgacagta (SEQ ID NO: 38) | |
| mVEGFR2-siRNA-25-a | 1393 | ggaaggcccauugaguccaacuaca (SEQ ID NO: 17) | 52 |
| mVEGFR2-siRNA-25-b | 1704 | ccaaacaagcccguaugcuuguaaa (SEQ ID NO: 64) | 44 |
| mVEGFR2-siRNA-25-c | 2587 | ggcacugcagugauugccauguucu (SEQ ID NO: 65) | 52 |

TABLE 8

| | NAME | SEQUENCE |
|---|---|---|
| 1 | hVEGF-25-siRNA-a | CCUGAUGAGAUCGAGUACAUCUUCA |
| 3 | hVEGF-25-siRNA-b | GAGAGAUGAGCUUCCUACAGCACAA |
| 5 | hVEGF-25-siRNA-c | CACAACAAAUGUGAAUGCAGACCAA |
| 9 | hVEGF-siRNA-a | UCGAGACCCUGGUGGACAU |
| 13 | hVEGFR2-25-siRNA-c | CCAAGUGAUUGAAGCAGAUGCCUUU |
| 14 | VEGF-2 | GAGUCCAACAUCACCAUGCAGAUUA |
| 15 | VEGF-3 | AGUCCAACAUCACCAUGCAGAUUAU |
| 16 | VEGF-4 | CCAACAUCACCAUGCAGAUUAUGCG |
| 17 | VEGF-5 | CACCAUGCAGAUUAUGCGGAUCAAA |
| 18 | VEGF-6 | GCACAUAGGAGAGAUGAGCUUCCUA |
| 19 | VEGFR2-1 | CCUCGGUCAUUUAUGUCUAUGUUCA |
| 20 | VEGFR2-2 | CAGAUCUCCAUUUAUUGCUUCUGUU |
| 21 | VEGFR2-3 | GACCAACAUGGAGUCGUGUACAUUA |
| 22 | VEGFR2-4 | CCCUUGAGUCCAAUCACACAAUUAA |
| 23 | VEGFR2-5 | CCAUGUUCUUCUGGCUACUUCUUGU |
| 24 | VEGFR2-6 | UCAUUCAUAUUGGUCACCAUCUCAA |
| 25 | VEGFR2-7 | GAGUUCUUGGCAUCGCGAAAGUGUA |
| 26 | VEGFR2-8 | CAGCAGGAAUCAGUCAGUAUCUGCA |
| 27 | VEGFR2-9 | CAGUGGUAUGGUUCUUGCCUCAGAA |
| 28 | VEGFR2-10 | CCACACUGAGCUCUCCUCCUGUUUA |
| 29 | VEGFR1-1 | CAAAGGACUUUAUACUUGUCGUGUA |
| 30 | VEGFR1-2 | CCCUCGCCGGAAGUUGUAUGGUUAA |
| 31 | VEGFR1-3 | CAUCACUCAGCGCAUGGCAAUAAUA |
| 32 | VEGFR1-4 | CCACCACUUUAGACUGUCAUGCUAA |
| 33 | VEGFR1-5 | CGGACAAGUCUAAUCUGGAGCUGAU |
| 34 | VEGFR1-6 | UGACCCACAUUGGCCACCAUCUGAA |
| 35 | VEGFR1-7 | GAGGGCCUCUGAUGGUGAUUGUUGA |
| 36 | VEGFR1-8 | CGAGCUCCGGCUUUCAGGAAGAUAA |
| 37 | VEGFR1-9 | CAAUCAAUGCCAUACUGACAGGAAA |
| 38 | VEGFR1-10 | GAAAGUAUUUCAGCUCCGAAGUUUA |
| 59 | hVEGFR2-25-siRNA-a | CCUCUUCUGUAAGACACUCACAAUU |
| 61 | hVEGFR2-25-siRNA-b | UUAAUUGUGUGAUUGGACUCAAGGG |
| 62 | hVEGFR2-25-siRNA-c | AAAGGCAUCUGCUUCAAUCACUUGG |
| 63 | hVEGFR1-25-siRNA-a | GCCAACAUAUUCUACAGUGUUCUUA |
| 65 | hVEGFR1-25-siRNA-b | UUAACCAUACAACUUCCGGCGAGGG |
| 66 | hVEGFR1-25-siRNA-c | CCUCAAGAGCAAACGUGACUUAUUU |

Table 8 shows siRNA sequences targeting VEGF pathway genes and discloses SEQ ID NOS 1-3, 66, 10, 67-74, 9, 75-81, 6, 82-89, 8, 90-91, 5, 92 and 43, respectively, in order of appearance.

TABLE 9
siRNA sequences targeting VEGF pathway genes

Human VEGF specific siRNA sequences
(25 basepairs with blunt ends):
VEGF-1, CCUGAUGAGAUCGAGUACAUCUUCA (SEQ ID NO: 1)
VEGF-2, GAGUCCAACAUCACCAUGCAGAUUA (SEQ ID NO: 67)
VEGF-3, AGUCCAACAUCACCAUGCAGAUUAU (SEQ ID NO: 68)
VEGF-4, CCAACAUCACCAUGCAGAUUAUGCG (SEQ ID NO: 69)
VEGF-5, CACCAUGCAGAUUAUGCGGAUCAAA (SEQ ID NO: 70)
VEGF-6, GCACAUAGGAGAGAUGAGCUUCCUA (SEQ ID NO: 71)
VEGF-7, GAGAGAUGAGCUUCCUACAGCACAA (SEQ ID NO: 2)
Human VEGFR1 specific siRNA sequences
(25 basepairs with blunt ends):
VEGFR1-1, CAAAGGACUUUAUACUUGUCGUGUA (SEQ ID NO:
81)
VEGFR1-2, CCCUCGCCGGAAGUUGUAUGGUUAA (SEQ ID NO: 6)
VEGFR1-3, CAUCACUCAGCGCAUGGCAAUAAUA (SEQ ID NO:
82)
VEGFR1-4, CCACCACUUUAGACUGUCAUGCUAA (SEQ ID NO:
83)
VEGFR1-5, CGGACAAGUCUAAUCUGGAGCUGAU (SEQ ID NO:
84)
VEGFR1-6, UGACCCACAUUGGCCACCAUCUGAA (SEQ ID NO:
85)
VEGFR1-7, GAGGGCCUCUGAUGGUGAUUGUUGA (SEQ ID NO:
86)
VEGFR1-8, CGAGCUCCGGCUUUCAGGAAGAUAA (SEQ ID NO:
87)
VEGFR1-9, CAAUCAAUGCCAUACUGACAGGAAA (SEQ ID NO:
88)
VEGFR1-10, GAAAGUAUUUCAGCUCCGAAGUUUA (SEQ ID NO:
89)
Human VEGFR2 specific siRNA sequences
(25 basepairs with blunt ends):
VEGFR2-1, CCUCGGUCAUUUAUGUCUAUGUUCA (SEQ ID NO:
72)
VEGFR2-2, CAGAUCUCCAUUUAUUGCUUCUGUU (SEQ ID NO:
73)
VEGFR2-3, GACCAACAUGGAGUCGUGUACAUUA (SEQ ID NO:
74)
VEGFR2-4, CCCUUGAGUCCAAUCACACAAUUAA (SEQ ID NO: 9)
VEGFR2-5, CCAUGUUCUUCUGGCUACUUCUUGU (SEQ ID NO:
75)
VEGFR2-6, UCAUUCAUAUUGGUCACCAUCUCAA (SEQ ID NO:
76)
VEGFR2-7, GAGUUCUUGGCAUCGCGAAAGUGUA (SEQ ID NO:
77)
VEGFR2-8, CAGCAGGAAUCAGUCAGUAUCUGCA (SEQ ID NO:
78)
VEGFR2-9, CAGUGGUAUGGUUCUUGCCUCAGAA (SEQ ID NO:
79)
VEGFR2-10, CCACACUGAGCUCUCCUCCUGUUUA (SEQ ID NO:
80)

TABLE 10
siRNA sequences targeting VEGF pathway genes
a. Human VEGF specific siRNA:

| | | |
|---|---|---|
| 25 base pair blunt ends: | | |
| hVEGF-25-siRNA-a: | | |
| Sense strand: | 5′-r(CCUGAUGAGAUCGAGUACAUCUUCA)-3′ | (SEQ ID NO: 1) |
| Antisense strand: | 5′-r(UGAAGAUGUACUCGAUCUCAUCAGG)-3′. | |
| hVEGF-25-siRNA-b: | | |
| Sense strand: | 5′-r(GAGAGAUGAGCUUCCUACAGCACAA)-3′ | (SEQ ID NO: 2) |
| Antisense strand: | 5′-r(UUGUGCUGUAGGAAGCUCAUCUCUC)-3′ | |
| hVEGF-25-siRNA-c: | | |
| Sense strand: | 5′-r(CACAACAAAUGUGAAUGCAGACCAA)-3′ | (SEQ ID NO: 3) |
| Antisense strand: | 5′-r(UUGGUCUGCAUUCACAUUUGUUGUG)-3′ | |
| hVEGF165 | 19 basepairs with two nucleotide overhangs at 3′: | |
| Sense strand: | 5′-r (UCGAGACCCUGGUGGACAUTT)-3′ | (SEQ ID NO: 4) |
| Antisense strand: | 5′-r (AUGUCCACCAGGGUCUCGATT)-3′ | (SEQ ID NO: 34) |

b. Human VEGF receptor 1 specific siRNA:

| | | |
|---|---|---|
| 25 base pair blunt ends: | | |
| hVEGFR1-25-siRNA-a, | | |
| Sense strand: | 5′-r(GCCAACAUAUUCUACAGUGUUCUUA)-3′ | (SEQ ID NO: 5) |
| Antisense strand: | 5′-r(UAAGAACACUGUAGAAUAUGUUGGC)-3′ | |
| hVEGFR1-25-siRNA-b, | | |
| Sense strand: | 5′-r(CCCUCGCCGGAAGUUGUAUGGUUAA)-3′ | (SEQ ID NO: 6) |
| Antisense strand: | 5′-r(UUAACCAUACAACUUCCGGCGAGGG)-3′. | (SEQ ID NO: 92) |

19 basepairs with 2 3′ (TT) nucleotide overhangs:

| | | |
|---|---|---|
| VEGF R1 (FLT) | 5′-GGAGAGGACCUGAAACUGUTT | (SEQ ID NO: 7) |

c. Human VEGF receptor 2 specific siRNA:

| | | |
|---|---|---|
| 25 basepair blunt ends: | | |
| hVEGFR2-25-siRNA-a, | | |
| Sense strand: | 5′-r(CCUCUUCUGUAAGACACUCACAAUU)-3′ | (SEQ ID NO: 8) |
| Antisense strand: | 5′-r(AAUUGUGAGUGUCUUACAGAAGAGG)-3′. | |
| hVEGFR2-25-siRNA-b, | | |
| Sense strand: | 5′-r(CCCUUGAGUCCAAUCACACAAUUAA)-3′ | (SEQ ID NO: 9) |
| Antisense strand: | 5′-r(UUAAUUGUGUGAUUGGACUCAAGGG)-3′. | (SEQ ID NO: 90) |
| hVEGFR2-25-siRNA-c, | | |
| Sense strand: | 5′-r(CCAAGUGAUUGAAGCAGAUGCCUUU)-3′ | (SEQ ID NO: 10) |
| Antisense strand: | 5′-r(AAAGGCAUCUGCUUCAAUCACUUGG)-3′ | (SEQ ID NO: 91) |

19 basepairs with 2 3′ (TT) nucleotide overhangs:

| | | |
|---|---|---|
| hVEGF R2 (KDR) | 5′-CAGUAAGCGAAAGAGCCGGTT-3′ | (SEQ ID NO: 11) |

25 base pair VEGF siRNA targeting human, mouse, rat, macaque, dog VEGF
mRNA sequences:

| | | |
|---|---|---|
| mhVEGF25-1: | sense, 5′-CAAGAUCCGCAGACGUGUAAAUGUU-3′; | (SEQ ID NO: 20) |
| | antisense, 5′-AACAUUUACACGUCUGCGGAUCUUG-3′ | |
| mhVEGF25-2: | sense, 5′-GCAGCUUGAGUUAAACGAACGUACU-3′; | (SEQ ID NO: 21) |
| | antisense, 5′-AGUACGUUCGUUUAACUCAAGCUGC-3′ | |
| mhVEGF25-3: | sense, 5′-CAGCUUGAGUUAAACGAACGUACUU-3′; | (SEQ ID NO: 48) |
| | antisense, 5′-AAGUACGUUCGUUUAACUCAAGCUG-3′ | |
| mhVEGF25-4: | sense, 5′-CCAUGCCAAGUGGUCCCAGGCUGCA-3′; | (SEQ ID NO: 22) |
| | antisense, 5′-TGCAGCCTGGGACCACTTGGCATGG-3′ | |
| mhVEGF25-4: | sense, 5′-CACAUAGGAGAGAUGAGCUUCCUCA-3′; | (SEQ ID NO: 94) |
| | antisense, 5′-UGAGGAAGCUCAUCUCUCCUAUGUG-3′ | |

25 base pair VEGF R2 siRNA sequences targeting both human and mouse
VEGFR2 mRNA sequences:

| | | |
|---|---|---|
| mhVEGFR225-1: | sense, 5′-CCUACGGACCGUUAAGCGGGCCAAU-3′; | (SEQ ID NO: 95) |
| | antisense: 5′-AUUGGCCCGCUUAACGGUCCGUAGG-3′ | |
| mhVEGFR225-2: | sense, 5′-CUCAUGUCUGUUCUCAAGAUCCUCA-3′; | (SEQ ID NO: 96) |
| | antisense: 5′-UGAGGAUCUUGAGAACAGACAUGAG-3′ | |
| mhVEGFR225-3: | sense, 5′-CUCAUGGUGAUUGUGGAAUUCUGCA-3′; | (SEQ ID NO: 97) |
| | antisense: 5′-UGCAGAAUUCCACAAUCACCAUGAG-3′ | |
| mhVEGFR225-4: | sense, 5′-GAGCAUGGAAGAGGAUUCUGGACUC-3′; | (SEQ ID NO: 98) |
| | antisense: 5′-GAGUCCAGAAUCCTCUUCCAUGCTC-3′ | |
| mhVEGFR225-5: | sense, 5′-CAGAACAGUAAGCGAAAGAGCCGGC-3′; | (SEQ ID NO: 99) |
| | antisense: 5′-GCCGGCUCUUUCGCUUACUGUUCUG-3′ | |
| mhVEGFR225-6: | sense, 5′-GACUUCCUGACCUUGGAGCAUCUCA-3′; | (SEQ ID NO: 29) |
| | antisense: 5′-UGAGAUGCUCCAAGGUCAGGAAGUC-3′ | |
| mhVEGFR225-7: | sense, 5′-CCUGACCUUGGAGCAUCUCAUCUGU-3′; | (SEQ ID NO: 30) |
| | antisense: 5′-ACAGAUGAGAUGCUCCAAGGUCAGG-3′ | |
| mhVEGFR225-8: | sense, 5′-GCUAAGGGCAUGGAGUUCUUGGCAU-3′; | (SEQ ID NO: 31) |
| | antisense: 5′-AUGCCAAGAACUCCAUGCCCUUAGC-3′ | |

25 base pairs VEGF R1 siRNA sequences targeting both human and mouse
VEGFR1 mRNA sequences:

| | | |
|---|---|---|
| mhVEGFR125-1: | sense, 5′-CACGCUGUUUAUUGA AAGAGUCACA-3′; | (SEQ ID NO: 100) |
| | antisense: 5′-UGUGACUCUUUCAAUAAACAGCGUG-3′ | |
| mhVEGFR125-2: | sense, 5′-CGCUGUUUAUUGAAAGAGUCACAGA-3′; | (SEQ ID NO: 50) |
| | antisense: 5′-UCUGUGACUCUUUCAAUAAACAGCG-3′ | |
| mhVEGFR125-3: | sense, 5′-CAAGGAGGGCCUCUGAUGGUGAUGU-3′; | (SEQ ID NO: 101) |
| | antisense: 5′-ACAUCACCAUCAGAGGCCCUCCUUG-3′ | |
| mhVEGFR125-4: | sense, 5′-CCAACUACCUCAAGAGCAAACGUGA-3′; | (SEQ ID NO: 24) |
| | antisense: 5′-UCACGUUUGCUCUUGAGGUAGUUGG-3′ | |
| mhVEGFR125-5: | sense, 5′-CUACCUCAAGAGCAAACGUGACUUA-3′; | (SEQ ID NO: 25) |
| | antisense: 5′-UAAGUCACGUUUGCUCUUGAGGUAG-3′ | |
| mhVEGFR125-6: | sense, 5′-CCAGAAAGUGCAUUCAUCGGGACCU-3′; | (SEQ ID NO: 26) |
| | antisense: 5′-AGGUCCCGAUGAAUGCACUUUCUGG-3′ | |
| mhVEGFR125-7: | sense, 5′-CAUUCAUCGGGACCUGGCAGCGAGA-3′; | (SEQ ID NO: 102) |
| | antisense: 5′-UCUCGCUGCCAGGUCCCGAUGAAUG-3′ | |
| mhVEGFR125-8: | sense, 5′-CAUCGGGACCUGGCAGCGAGAAACA-3′; | (SEQ ID NO: 103) |
| | antisense: 5′-UGUUUCUCGCUGCCAGGUCCCGAUG-3′ | |
| mhVEGFR125-9: | sense, 5′-GAGCCUGGAAAGAAUCAAAACCUUU-3′; | (SEQ ID NO: 104) |
| | antisense: 5′-AAAGGUUUUGAUUCUUUCCAGGCUC-3′ | |
| mhVEGFR125-10: | sense, 5′-GCCUGGAAAGAAUCAAAACCUUUGA-3′; | (SEQ ID NO: 105) |
| | antisense: 5′-UCAAAGGUUUUGAUUCUUUCCAGGC-3′ | |
| mhVEGFR125-11: | sense, 5′-GCCUGGAAAGAAUCAAAACCUUUGA-3′; | (SEQ ID NO: 105) |
| | antisense: 5′-UCAAAGGUUUUGAUUCUUUCCAGGC-3′ | |
| mhVEGFR125-12: | sense, 5′-CUGAACUGAGUUUAAAAGGCACCCA-3′; | (SEQ ID NO: 106) |
| | antisense: 5′-UGGGUGCCUUUUAAACUGAGUUCAG-3′ | |
| mhVEGFR125-13: | sense, 5′-GAACUGAGUUUAAAAGGCACCCAGC-3′; | (SEQ ID NO: 107) |
| | antisense: 5′-GCUGGGUGCCUUUUAAACUCAGUUG-3′ | |

TABLE 11
siRNA sequences targeting VEGF pathway genes

| | | |
|---|---|---|
| No. | Target sequence | GC % |

25-mer hVEGF siRNAs

| | | |
|---|---|---|
| 1 | 5′-uaucagcgcagcuacugccauccaa-3′ (SEQ ID NO: 108) | 52 |
| 2 | 5′-gaguccaacaucaccaugcagauua-3′ (SEQ ID NO: 67) | 44 |
| 3 | 5′-ucaccaugcagauuaugcggaucaa-3′ (SEQ ID NO: 109) | 44 |
| 4 | 5′-gcacauaggagagaugagcuuccua-3′ (SEQ ID NO: 71) | 48 |
| 5 | 5′-agaugagcuuccuacagcacaacaa-3′ (SEQ ID NO: 110) | 44 |
| 6 | 5′-acaacaaaugugaaugcagaccaaa-3′ (SEQ ID NO: 111) | 36 |
| 7 | 5′-acaaaugugaaugcagaccaaagaa-3′ (SEQ ID NO: 112) | 36 |

25-mer hVEGFR1 siRNAs

| | | |
|---|---|---|
| 1 | 5′-ggagcacuccaucacucuuaaucuu-3′ (SEQ ID NO: 113) | 44 |
| 2 | 5′-gguucaagcaucagcauuuggcauu-3′ (SEQ ID NO: 114) | 44 |
| 3 | 5′-gcauuuggcauuaagaaaucaccua-3′ (SEQ ID NO: 115) | 36 |
| 4 | 5′-gcaaauauggaaaucucuccaacua-3′ (SEQ ID NO: 116) | 36 |
| 5 | 5′-ccaagauuugcagaacuuguggaaa-3′ (SEQ ID NO: 117) | 40 |
| 6 | 5′-gguuuacauacucaacuccugccuu-3′ (SEQ ID NO: 118) | 44 |
| 7 | 5′-ggaaaguauuucagcuccgaaguuu-3′ (SEQ ID NO: 119) | 40 |

25-mer hVEGFR2 siRNAs

| | | |
|---|---|---|
| 1 | 5′-ggaaacugacuuggccucggucauu-3′ (SEQ ID NO: 120) | 52 |
| 2 | 5′-ggccucggucauuuaugucuauguu-3′ (SEQ ID NO: 121) | 44 |
| 3 | 5′-gguucugaguccgucucauggaauu-3′ (SEQ ID NO: 122) | 48 |
| 4 | 5′-ggaccaaggagacuaugucugccuu-3′ (SEQ ID NO: 123) | 52 |
| 5 | 5′-cccuccacagaucaugugguuuaaa-3′ (SEQ ID NO: 124) | 44 |
| 6 | 5′-ggugauuguggaauucugcaaauuu-3′ (SEQ ID NO: 125) | 36 |
| 7 | 5′-ggaacauuugggaaaucucuugcaa-3′ (SEQ ID NO: 126) | 40 |

TABLE 12
Negative control siRNA sequences

| | |
|---|---|
| Luc-25-siRNA | 5′-GGAACCGCUGGAGAGCAACUGCAUA-3′ (SEQ ID NO: 32) |
| | (sense strand) |
| | 5′-CCUUGGCGACCUCUCGUUGACGUAU-3′ (SEQ ID NO: 33) |
| | (antisense strand) |
| GFP | 5′-GCUGACCCUGAAGUUCAUCdTT-3′ (SEQ ID NO: 35) |
| | (sense strand) |
| | 5′-GAUGAACUUCAGGGUCAGCdTT-3′ (SEQ ID NO: 36) |
| | (antisense strand) |
| GFP-21-a | 5′-AAGCUGACCCUGAAGUUCAUC-3′ (SEQ ID NO: 18) |
| GFP-21-b | 5′-AAGCAGCACGACUUCUUCAAG-3′ (SEQ ID NO: 19) |

### REFERENCES

1. Peter A, Campochiaro. Retinal and choroidal neovascularization. J Cell Physiol, 2000, 184: 301-10.
2. Smith L E, Wesolowski E, Mclellan A, et al. Oxygen-induced retinopathy in the mouse. Invest Opthalmol V is Sci, 1994, 35: 101-11.
3. 3 D'Amato R, Wesolowski E, Smith L E. Microscopic visualization of the retina by angiography with high-molecular-weight fluorescein-labeled dextrans in the mouse. Microvasc Res 1993; 46:135-42.
4. Jikui shen, Rebecca samul, Joelle Zimmer, et al. Deficiency of Neuropilin 2 Suppresses VEGF-Induced Retinal NV Molecular medicine, 2004, 10:12-18.
5. Unsoeld A S, Junker B, Mazitschek R, et al. Local injection of receptor tyrosine kinase inhibitor MAE 87 reduces retinal neovascularization in mice. Mol Vis, 2004, 10:468-75.
6. Clauss M. Molecular biology of the VEGF and VEGF receptor family. Semin Thromb Hemost, 2000, 26: 561-9.
7. Eric A P, Robert L A, Eliot D F, et al. Vascular endothelial growth factor/vascular permeability factor expression in a mouse model of retinal neovascularization. Proc. Natl. Acad. Sci, 1995, 92:905-9.
8. Lu P. Y. et al., Keystone Symposia, Molecular Targets for Cancer Therapy, (2003, p 219. Xu J. et al., Gene Suppression. (2003).
9. Lu, Patrick et al., (2002), Cancer Gene Therapy, Vol. 10, Supplement 1, 011.
10. Lu, Patrick Y et al., (2003), Current Opinion in Molecular Therapeutics, 5(3):225-234.
11. Cogoni C. et al., (2000), Genes Dev 10: 638-643.
12. Guru T., (2000), Nature 404: 804-808.
13. Hammond S M et al., (2001), Nature Rev Gen 2: 110-119.
14. Napoli C et al., (1990), Plant Cell 2: 279-289.
15. Jorgensen R A et al., (1996), Plant Mol Biol, 31: 957-973.
16. Ingelbrecht I et al., (1994), Proc Natl Acad Sci USA, 91: 10502-10506.
17. Cogoni C et al., EMBO J, 15: 3153-3163.
18. Palauqui J C et al., (1998), EMBO J, 16: 4738-4745.
19. Guo S et al., (1995), Cell, 81: 611-620.
20. Fire A et al., (1998), Nature 391: 806-811.
21. Timmons L. et al., (1998), Nature 395: 854.
22. Timmons L et al., (2001), Gene 263:103-112.
23. Hunter C P, (2000), Current Biology 10: R137-R140.
24. Tabara H et al., (1998), Science 282: 430-431.
25. Kamath R S et al., (2000), Genome Biology 2: 2.1-2.10.
26. Grishok A et al., (2000), Science 287: 2494-2497.
27. Sharp P A et al., (2000), Science 287: 2431-2433.
28. Sharp P A, (2001), Genes Dev 15: 485-490.
29. Kennerdell J R et al., (1998). Cell 95: 1017-1026.
30. Kennerdell J R et al., (2000), Nature Biotech 18: 896-898.
31. Dzitoyeva S et al., (2001), Mol Psychiatry 6(6):665-670.
32. Worby C A et al., (2001), Sci STKE Aug. 14, 2001(95):PL1.
33. Schmid A et al., (2002), Trends Neurosci 25(2):71-74.)
34. Hamilton A. J. et al., (1999), Science 286: 950-952.
35. Hammond S et al., (2000), Nature, 404: 293-298.
36. Zamore P D et al., (2000), Cell 101: 25-33.
37. Hutvagner G et al., (2002), Curr Opin Genetics & Development 12:225-232.
38. Bernstein E et al., (2001), Nature 409:363-366.
39. Nykanen A et al., (2001), Cell 107:309-321.
40. Lipardi C et al., (2001), Cell 107:297-307.
41. Ketting R F et al., (1999), Cell 99: 133-141.
42. Grishok A et al., (2001), Cell 106:23-34.
43. Hutvagner G et al., (2001), Science 293(5531):834-838.
44. Ketting R F et al., (2001), Genes Dev 15(20):2654-2659.
45. Lagos-Quintana M et al., (2001), Science 294:853-858.
46. Lau N C et al., (2001), Science 294:858-862.
47. Lee R C et al., (2001), Science 294:862-864.
48. Ruvkun G., (2001), Science 294:797-799.
49. Manche L et al., (1992), Mol. Cell. Biol. 12:5238-5248.
50. Minks M A et al., (1979), J. Biol. Chem. 254:10180-10183.
51. Yang S et al., (2001), Mol. Cell. Biol. 21(22):7807-7816.
52. Paddison P J et al., (2002), Proc. Natl. Acad. Sci. USA 99(3):1443-1448.
53. Elbashir S M et al., (2001), Genes Dev 15(2):188-200.
54. Elbashir S M et al., (2001), Nature 411: 494-498.
55. Caplen N J et al., (2001), Proc. Natl. Acad. Sci. USA 98: 9746-9747.
56. Holen T et al., (2002), Nucleic Acids Research 30(8):1757-1766.
57. Elbashir S M et al., (2001), EMBO J 20: 6877-6888.
58. Jarvis R A et al., (2001), TechNotes 8(5): 3-5.
59. Brown D et al., (2002), TechNotes 9(1): 3-5.
60. Brummelkamp T R et al., (2002), Science 296:550-553.
61. Lee N S et al., (2002), Nature Biotechnol. 20:500-505.
62. Miyagishi M et al., (2002), Nature Biotechnol. 20:497-500.
63. Paddison P J et al., (2002), Genes & Dev. 16:948-958.
64. Paul C P et al., (2002), Nature Biotechnol. 20:505-508.
65. Sui G et al., (2002), Proc. Natl. Acad. Sci. USA 99(6):5515-5520.
66. Yu J-Y et al., (2002), Proc. Natl. Acad. Sci. USA 99(9):6047-6052.
67. McCaffrey, A. P. et al., (2002), Nature, Vol. 418, July, 2002.
68. Watanabe A, Taniguchi M. (2005). A case of oropharyngeal cancer with multiple bone metastases from prostate cancer that responded to docetaxel, ifosfamide and cisplatin combination therapy. Gan To Kagaku Ryoho. 32(1):65-7.
69. Miner J, Gillan M M, Alex P, Centola M. (2005). Steroid-Refractory Ulcerative Colitis Treated with Corticosteroids, Metronidazole and Vancomycin: a Case Report. BMC Gastroenterol. 5(1):3.
70. Orbay E, Sargin M, Sargin H, Gozu H, Bayramicli O U, Yayla A. (2004). Addition of rosiglitazone to glimepirid and metformin combination therapy in type 2 diabetes. Endocr J. 51(6):521-7.

1. An antisense nucleic acid molecule for targeting VEGFR1, wherein the antisense nucleic acid comprises a nucleotide sequence that is complementary to a sense strand nucleotide sequence selected from the group consisting of SEQ ID NOs: 129-196.

2. A double-stranded nucleic acid molecule comprising the antisense nucleic acid molecule of claim 1 and its corresponding sense strand.

3. An antisense nucleic acid molecule for targeting VEGFR1, wherein the antisense nucleic acid molecule comprises a sequence that is complementary to a sense strand nucleotide sequence selected from the group consisting of SEQ ID NOs: 24, 49, 50, 51, 84, 85, 86, 88, 89, 100, 104, 105, 184, 186, 188, 192, 193, 194, and 196.

4. (canceled)

5. The antisense nucleic acid molecule of claim 3, wherein the antisense nucleic acid molecule comprises a sequence that is complementary to VEGFR1 mRNA and wherein the nucleic acid molecule increases the expression of soluble VEGFR1 and decreases the expression of full-length VEGFR1 in a cell.

6. (canceled)

7. A composition comprising the nucleic acid molecule of claim 1 and a pharmaceutically acceptable carrier.

8. A synthetic nucleic acid delivery vehicle comprising the nucleic acid molecule of claim 1.

9. The composition of claim 7 which comprises a cationic polymer-nucleic acid complex.

10. The synthetic nucleic acid delivery vehicle of claim 9, wherein the cationic polymer is PEI or a histidine-lysine copolymer.

11. A method for reducing total VEGR1 expression in a cell, comprising the step of contacting the cell with the nucleic acid molecule of claim 1.

12. (canceled)

13. A method for increasing soluble VEGFR1 expression in a cell, comprising the step of contacting the cell with an antisense nucleic acid molecule of claim 3.

14-19. (canceled)

20. A method for reducing full-length VEGR1 expression and increasing soluble VEGFR1 expression in a cell, comprising the step of contacting the cell with an antisense nucleic acid molecule of claim 3.

21. A method for reducing neovascularization in a subject in need thereof, comprising the step of administering to the subject an antisense nucleic acid molecule of claim 3.

22. The method of claim 21, wherein the neovascularization is in a tumor.

23. The nucleic acid molecule of claim 1 which comprises at least one modified nucleotide or rare nucleotide.

24. The nucleic acid molecule of claim 1 which comprises at least one chemically modified nucleotide.

25. The double-stranded nucleic acid molecule of claim 2 which comprises one or more mismatched base pairs.

26. The double-stranded nucleic acid molecule of claim 2 which is a double-stranded small interfering RNA (siRNA) with blunt ends.

27. The composition of claim 7 which comprises a hydrophilic polymer.

28. The composition of claim 7 which comprises a targeting moiety.

29. The composition of claim 7 comprising an additional therapeutic agent.


SEQUENCE LISTING (321 sequences)

SEQ ID NO: 1 - RNA, 25 nt, Artificial Sequence
  source: /note=“Description of Artificial Sequence: Synthetic oligonucleotide”
ccugaugagaucgaguacaucuuca

SEQ ID NO: 2 - RNA, 25 nt, Artificial Sequence
  source: /note=“Description of Artificial Sequence: Synthetic oligonucleotide”
gagagaugagcuuccuacagcacaa

SEQ ID NO: 3 - RNA, 25 nt, Artificial Sequence
  source: /note=“Description of Artificial Sequence: Synthetic oligonucleotide”
cacaacaaaugugaaugcagaccaa

SEQ ID NO: 4 - DNA, 21 bp, Artificial Sequence
  source: /note=“Description of Artificial Sequence: Synthetic oligonucleotide”
ucgagacccugguggacautt

SEQ ID NO: 5 - RNA, 25 nt, Artificial Sequence
  source: /note=“Description of Artificial Sequence: Synthetic oligonucleotide”
gccaacauauucuacaguguucuua

SEQ ID NO: 6 - RNA, 25 nt, Artificial Sequence
  source: /note=“Description of Artificial Sequence: Synthetic oligonucleotide”
cccucgccggaaguuguaugguuaa

SEQ ID NO: 7 - DNA, 21 bp, Artificial Sequence
  source: /note=“Description of Combined DNA/RNA Molecule: Synthetic oligonucleotide”
  source: /note=“Description of Artificial Sequence: Synthetic oligonucleotide”
ggagaggaccugaaacugutt

SEQ ID NO: 8 - RNA, 25 nt, Artificial Sequence
  source: /note=“Description of Artificial Sequence: Synthetic oligonucleotide”
ccucuucuguaagacacucacaauu

SEQ ID NO: 9 - RNA, 25 nt, Artificial Sequence
  source: /note=“Description of Artificial Sequence: Synthetic oligonucleotide”
cccuugaguccaaucacacaauuaa

SEQ ID NO: 10 - RNA, 25 nt, Artificial Sequence
  source: /note=“Description of Artificial Sequence: Synthetic oligonucleotide”
ccaagugauugaagcagaugccuuu

SEQ ID NO: 11 - DNA, 21 bp, Artificial Sequence
  source: /note=“Description of Combined DNA/RNA Molecule: Synthetic oligonucleotide”
  source: /note=“Description of Artificial Sequence: Synthetic oligonucleotide”
caguaagcgaaagagccggtt

SEQ ID NO: 12 - protein, 10 aa, Artificial Sequence
  source: /note=“Description of Artificial Sequence: Synthetic peptide”
ACRGDMFGCA

SEQ ID NO: 13 - RNA, 25 nt, Artificial Sequence
  source: /note=“Description of Artificial Sequence: Synthetic oligonucleotide”
gcaaggcgaggcagcuugaguuaaa

SEQ ID NO: 14 - RNA, 25 nt, Artificial Sequence
  source: /note=“Description of Artificial Sequence: Synthetic oligonucleotide”
ccgcagacguguaaauguuccugca

SEQ ID NO: 15 - RNA, 25 nt, Artificial Sequence
  source: /note=“Description of Artificial Sequence: Synthetic oligonucleotide”
gcagacguguaaauguuccugcaaa

SEQ ID NO: 16 - RNA, 25 nt, Artificial Sequence
  source: /note=“Description of Artificial Sequence: Synthetic oligonucleotide”
gcggaaucuucaaucuacauauuug

SEQ ID NO: 17 - RNA, 25 nt, Artificial Sequence
  source: /note=“Description of Artificial Sequence: Synthetic oligonucleotide”
ggaaggcccauugaguccaacuaca

SEQ ID NO: 18 - RNA, 21 nt, Artificial Sequence
  source: /note=“Description of Artificial Sequence: Synthetic oligonucleotide”
aagcugacccugaaguucauc

SEQ ID NO: 19 - RNA, 21 nt, Artificial Sequence
  source: /note=“Description of Artificial Sequence: Synthetic oligonucleotide”
aagcagcacgacuucuucaag

SEQ ID NO: 20 - RNA, 25 nt, Artificial Sequence
  source: /note=“Description of Artificial Sequence: Synthetic oligonucleotide”
caagauccgcagacguguaaauguu

SEQ ID NO: 21 - RNA, 25 nt, Artificial Sequence
  source: /note=“Description of Artificial Sequence: Synthetic oligonucleotide”
gcagcuugaguuaaacgaacguacu

SEQ ID NO: 22 - RNA, 25 nt, Artificial Sequence
  source: /note=“Description of Artificial Sequence: Synthetic oligonucleotide”
ccaugccaaguggucccaggcugca

SEQ ID NO: 23 - RNA, 25 nt, Artificial Sequence
  source: /note=“Description of Artificial Sequence: Synthetic oligonucleotide”
cccugguggacaucuuccaggagua

SEQ ID NO: 24 - RNA, 25 nt, Artificial Sequence
  source: /note=“Description of Artificial Sequence: Synthetic oligonucleotide”
ccaacuaccucaagagcaaacguga

SEQ ID NO: 25 - RNA, 25 nt, Artificial Sequence
  source: /note=“Description of Artificial Sequence: Synthetic oligonucleotide”
cuaccucaagagcaaacgugacuua

SEQ ID NO: 26 - RNA, 25 nt, Artificial Sequence
  source: /note=“Description of Artificial Sequence: Synthetic oligonucleotide”
ccagaaagugcauucaucgggaccu

SEQ ID NO: 27-28 - skipped (2 entries)

SEQ ID NO: 29 - RNA, 25 nt, Artificial Sequence
  source: /note=“Description of Artificial Sequence: Synthetic oligonucleotide”
gacuuccugaccuuggagcaucuca

SEQ ID NO: 30 - RNA, 25 nt, Artificial Sequence
  source: /note=“Description of Artificial Sequence: Synthetic oligonucleotide”
ccugaccuuggagcaucucaucugu

SEQ ID NO: 31 - RNA, 25 nt, Artificial Sequence
  source: /note=“Description of Artificial Sequence: Synthetic oligonucleotide”
gcuaagggcauggaguucuuggcau

SEQ ID NO: 32 - RNA, 25 nt, Artificial Sequence
  source: /note=“Description of Artificial Sequence: Synthetic oligonucleotide”
ggaaccgcuggagagcaacugcaua

SEQ ID NO: 33 - RNA, 25 nt, Artificial Sequence
  source: /note=“Description of Artificial Sequence: Synthetic oligonucleotide”
ccuuggcgaccucucguugacguau

SEQ ID NO: 34 - DNA, 21 bp, Artificial Sequence
  source: /note=“Description of Combined DNA/RNA Molecule: Synthetic oligonucleotide”
  source: /note=“Description of Artificial Sequence: Synthetic oligonucleotide”
auguccaccagggucucgatt

SEQ ID NO: 35 - DNA, 21 bp, Artificial Sequence
  source: /note=“Description of Combined DNA/RNA Molecule: Synthetic oligonucleotide”
  source: /note=“Description of Artificial Sequence: Synthetic oligonucleotide”
gcugacccugaaguucauctt

SEQ ID NO: 36 - DNA, 21 bp, Artificial Sequence
  source: /note=“Description of Combined DNA/RNA Molecule: Synthetic oligonucleotide”
  source: /note=“Description of Artificial Sequence: Synthetic oligonucleotide”
gaugaacuucagggucagctt

SEQ ID NO: 37 - DNA, 21 bp, Artificial Sequence
  source: /note=“Description of Artificial Sequence: Synthetic oligonucleotide”
aagctcagcacacagaaagac

SEQ ID NO: 38 - DNA, 21 bp, Artificial Sequence
  source: /note=“Description of Artificial Sequence: Synthetic oligonucleotide”
aatgcggcggtggtgacagta

SEQ ID NO: 39 - RNA, 25 nt, Artificial Sequence
  source: /note=“Description of Artificial Sequence: Synthetic oligonucleotide”
gggacaguaggagaggcuuuauaau

SEQ ID NO: 40 - DNA, 21 bp, Artificial Sequence
  source: /note=“Description of Artificial Sequence: Synthetic oligonucleotide”
aatcgagaccctggtggacat

SEQ ID NO: 41 - RNA, 25 nt, Artificial Sequence
  source: /note=“Description of Artificial Sequence: Synthetic oligonucleotide”
caaucgagacccugguggacaucuu

SEQ ID NO: 42 - RNA, 25 nt, Artificial Sequence
  source: /note=“Description of Artificial Sequence: Synthetic oligonucleotide”
aaucgagacccugguggacaucuuc

SEQ ID NO: 43 - RNA, 25 nt, Artificial Sequence
  source: /note=“Description of Artificial Sequence: Synthetic oligonucleotide”
ccucaagagcaaacgugacuuauuu

SEQ ID NO: 44 - RNA, 21 nt, Artificial Sequence
  source: /note=“Description of Artificial Sequence: Synthetic oligonucleotide”
aaucgagacccugguggacau

SEQ ID NO: 45 - RNA, 21 nt, Artificial Sequence
  source: /note=“Description of Artificial Sequence: Synthetic oligonucleotide”
aaggccagcacauaggagaga

SEQ ID NO: 46 - RNA, 25 nt, Artificial Sequence
  source: /note=“Description of Artificial Sequence: Synthetic oligonucleotide”
auccaaucgagacccugguggacau

SEQ ID NO: 47 - RNA, 25 nt, Artificial Sequence
  source: /note=“Description of Artificial Sequence: Synthetic oligonucleotide”
uccaaucgagacccugguggacauc

SEQ ID NO: 48 - RNA, 25 nt, Artificial Sequence
  source: /note=“Description of Artificial Sequence: Synthetic oligonucleotide”
cagcuugaguuaaacgaacguacuu

SEQ ID NO: 49 - RNA, 25 nt, Artificial Sequence
  source: /note=“Description of Artificial Sequence: Synthetic oligonucleotide”
gcuguuuauugaaagagucacagaa

SEQ ID NO: 50 - RNA, 25 nt, Artificial Sequence
  source: /note=“Description of Artificial Sequence: Synthetic oligonucleotide”
cgcuguuuauugaaagagucacaga

SEQ ID NO: 51 - RNA, 25 nt, Artificial Sequence
  source: /note=“Description of Artificial Sequence: Synthetic oligonucleotide”
accucaagagcaaacgugacuuauu

SEQ ID NO: 52 - RNA, 25 nt, Artificial Sequence
  source: /note=“Description of Artificial Sequence: Synthetic oligonucleotide”
caucucaucuguuacagcuuccaag

SEQ ID NO: 53 - RNA, 25 nt, Artificial Sequence
  source: /note=“Description of Artificial Sequence: Synthetic oligonucleotide”
cauggaagaggauucuggacucucu

SEQ ID NO: 54 - RNA, 25 nt, Artificial Sequence
  source: /note=“Description of Artificial Sequence: Synthetic oligonucleotide”
caaguggcuaagggcauggaguucu

SEQ ID NO: 55 - RNA, 25 nt, Artificial Sequence
  source: /note=“Description of Artificial Sequence: Synthetic oligonucleotide”
gggaacugaagacaggcuacuuguc

SEQ ID NO: 56 - RNA, 23 nt, Artificial Sequence
  source: /note=“Description of Artificial Sequence: Synthetic oligonucleotide”
gacuggcuuuggcccaauaauca

SEQ ID NO: 57 - RNA, 25 nt, Artificial Sequence
  source: /note=“Description of Artificial Sequence: Synthetic oligonucleotide”
cccgacgagauagaguacaucuuca

SEQ ID NO: 58 - RNA, 25 nt, Artificial Sequence
  source: /note=“Description of Artificial Sequence: Synthetic oligonucleotide”
ccgacgagauagaguacaucuucaa

SEQ ID NO: 59 - RNA, 21 nt, Artificial Sequence
  source: /note=“Description of Artificial Sequence: Synthetic oligonucleotide”
aaguuaaaagugccugaacug

SEQ ID NO: 60 - RNA, 21 nt, Artificial Sequence
  source: /note=“Description of Artificial Sequence: Synthetic oligonucleotide”
aagcaggccagacucucuuuc

SEQ ID NO: 61 - RNA, 25 nt, Artificial Sequence
  source: /note=“Description of Artificial Sequence: Synthetic oligonucleotide”
ugacccacaucggccaucaucugaa

SEQ ID NO: 62 - RNA, 21 nt, Artificial Sequence
  source: /note=“Description of Artificial Sequence: Synthetic oligonucleotide”
aagcucagcacacagaaagac

SEQ ID NO: 63 - RNA, 21 nt, Artificial Sequence
  source: /note=“Description of Artificial Sequence: Synthetic oligonucleotide”
aaugcggcgguggugacagua

SEQ ID NO: 64 - RNA, 25 nt, Artificial Sequence
  source: /note=“Description of Artificial Sequence: Synthetic oligonucleotide”
ccaaacaagcccguaugcuuguaaa

SEQ ID NO: 65 - RNA, 25 nt, Artificial Sequence
  source: /note=“Description of Artificial Sequence: Synthetic oligonucleotide”
ggcacugcagugauugccauguucu

SEQ ID NO: 66 - RNA, 19 nt, Artificial Sequence
  source: /note=“Description of Artificial Sequence: Synthetic oligonucleotide”
ucgagacccugguggacau

SEQ ID NO: 67 - RNA, 25 nt, Artificial Sequence
  source: /note=“Description of Artificial Sequence: Synthetic oligonucleotide”
gaguccaacaucaccaugcagauua

SEQ ID NO: 68 - RNA, 25 nt, Artificial Sequence
  source: /note=“Description of Artificial Sequence: Synthetic oligonucleotide”
aguccaacaucaccaugcagauuau

SEQ ID NO: 69 - RNA, 25 nt, Artificial Sequence
  source: /note=“Description of Artificial Sequence: Synthetic oligonucleotide”
ccaacaucaccaugcagauuaugcg

SEQ ID NO: 70 - RNA, 25 nt, Artificial Sequence
  source: /note=“Description of Artificial Sequence: Synthetic oligonucleotide”
caccaugcagauuaugcggaucaaa

SEQ ID NO: 71 - RNA, 25 nt, Artificial Sequence
  source: /note=“Description of Artificial Sequence: Synthetic oligonucleotide”
gcacauaggagagaugagcuuccua

SEQ ID NO: 72 - RNA, 25 nt, Artificial Sequence
  source: /note=“Description of Artificial Sequence: Synthetic oligonucleotide”
ccucggucauuuaugucuauguuca

SEQ ID NO: 73 - RNA, 25 nt, Artificial Sequence
  source: /note=“Description of Artificial Sequence: Synthetic oligonucleotide”
cagaucuccauuuauugcuucuguu

SEQ ID NO: 74 - RNA, 25 nt, Artificial Sequence
  source: /note=“Description of Artificial Sequence: Synthetic oligonucleotide”
gaccaacauggagucguguacauua

SEQ ID NO: 75 - RNA, 25 nt, Artificial Sequence
  source: /note=“Description of Artificial Sequence: Synthetic oligonucleotide”
ccauguucuucuggcuacuucuugu

SEQ ID NO: 76 - RNA, 25 nt, Artificial Sequence
  source: /note=“Description of Artificial Sequence: Synthetic oligonucleotide”
ucauucauauuggucaccaucucaa

SEQ ID NO: 77 - RNA, 25 nt, Artificial Sequence
  source: /note=“Description of Artificial Sequence: Synthetic oligonucleotide”
gaguucuuggcaucgcgaaagugua

SEQ ID NO: 78 - RNA, 25 nt, Artificial Sequence
  source: /note=“Description of Artificial Sequence: Synthetic oligonucleotide”
cagcaggaaucagucaguaucugca

SEQ ID NO: 79 - RNA, 25 nt, Artificial Sequence
  source: /note=“Description of Artificial Sequence: Synthetic oligonucleotide”
cagugguaugguucuugccucagaa

SEQ ID NO: 80 - RNA, 25 nt, Artificial Sequence
  source: /note=“Description of Artificial Sequence: Synthetic oligonucleotide”
ccacacugagcucuccuccuguuua

SEQ ID NO: 81 - RNA, 25 nt, Artificial Sequence
  source: /note=“Description of Artificial Sequence: Synthetic oligonucleotide”
caaaggacuuuauacuugucgugua

SEQ ID NO: 82 - RNA, 25 nt, Artificial Sequence
  source: /note=“Description of Artificial Sequence: Synthetic oligonucleotide”
caucacucagcgcauggcaauaaua

SEQ ID NO: 83 - RNA, 25 nt, Artificial Sequence
  source: /note=“Description of Artificial Sequence: Synthetic oligonucleotide”
ccaccacuuuagacugucaugcuaa

SEQ ID NO: 84 - RNA, 25 nt, Artificial Sequence
  source: /note=“Description of Artificial Sequence: Synthetic oligonucleotide”
cggacaagucuaaucuggagcugau

SEQ ID NO: 85 - RNA, 25 nt, Artificial Sequence
  source: /note=“Description of Artificial Sequence: Synthetic oligonucleotide”
ugacccacauuggccaccaucugaa

SEQ ID NO: 86 - RNA, 25 nt, Artificial Sequence
  source: /note=“Description of Artificial Sequence: Synthetic oligonucleotide”
gagggccucugauggugauuguuga

SEQ ID NO: 87 - RNA, 25 nt, Artificial Sequence
  source: /note=“Description of Artificial Sequence: Synthetic oligonucleotide”
cgagcuccggcuuucaggaagauaa

SEQ ID NO: 88 - RNA, 25 nt, Artificial Sequence
  source: /note=“Description of Artificial Sequence: Synthetic oligonucleotide”
caaucaaugccauacugacaggaaa

SEQ ID NO: 89 - RNA, 25 nt, Artificial Sequence
  source: /note=“Description of Artificial Sequence: Synthetic oligonucleotide”
gaaaguauuucagcuccgaaguuua

SEQ ID NO: 90 - RNA, 25 nt, Artificial Sequence
  source: /note=“Description of Artificial Sequence: Synthetic oligonucleotide”
uuaauugugugauuggacucaaggg

SEQ ID NO: 91 - RNA, 25 nt, Artificial Sequence
  source: /note=“Description of Artificial Sequence: Synthetic oligonucleotide”
aaaggcaucugcuucaaucacuugg

SEQ ID NO: 92 - RNA, 25 nt, Artificial Sequence
  source: /note=“Description of Artificial Sequence: Synthetic oligonucleotide”
uuaaccauacaacuuccggcgaggg

SEQ ID NO: 93 - skipped

SEQ ID NO: 94 - RNA, 25 nt, Artificial Sequence
  source: /note=“Description of Artificial Sequence: Synthetic oligonucleotide”
cacauaggagagaugagcuuccuca

SEQ ID NO: 95 - RNA, 25 nt, Artificial Sequence
  source: /note=“Description of Artificial Sequence: Synthetic oligonucleotide”
ccuacggaccguuaagcgggccaau

SEQ ID NO: 96 - RNA, 25 nt, Artificial Sequence
  source: /note=“Description of Artificial Sequence: Synthetic oligonucleotide”
cucaugucuguucucaagauccuca

SEQ ID NO: 97 - RNA, 25 nt, Artificial Sequence
  source: /note=“Description of Artificial Sequence: Synthetic oligonucleotide”
cucauggugauuguggaauucugca

SEQ ID NO: 98 - RNA, 25 nt, Artificial Sequence
  source: /note=“Description of Artificial Sequence: Synthetic oligonucleotide”
gagcauggaagaggauucuggacuc

SEQ ID NO: 99 - RNA, 25 nt, Artificial Sequence
  source: /note=“Description of Artificial Sequence: Synthetic oligonucleotide”
cagaacaguaagcgaaagagccggc

SEQ ID NO: 100 - RNA, 25 nt, Artificial Sequence
  source: /note=“Description of Artificial Sequence: Synthetic oligonucleotide”
cacgcuguuuauugaaagagucaca

SEQ ID NO: 101 - RNA, 25 nt, Artificial Sequence
  source: /note=“Description of Artificial Sequence: Synthetic oligonucleotide”
caaggagggccucugauggugaugu

SEQ ID NO: 102 - RNA, 25 nt, Artificial Sequence
  source: /note=“Description of Artificial Sequence: Synthetic oligonucleotide”
cauucaucgggaccuggcagcgaga

SEQ ID NO: 103 - RNA, 25 nt, Artificial Sequence
  source: /note=“Description of Artificial Sequence: Synthetic oligonucleotide”
caucgggaccuggcagcgagaaaca

SEQ ID NO: 104 - RNA, 25 nt, Artificial Sequence
  source: /note=“Description of Artificial Sequence: Synthetic oligonucleotide”
gagccuggaaagaaucaaaaccuuu

SEQ ID NO: 105 - RNA, 25 nt, Artificial Sequence
  source: /note=“Description of Artificial Sequence: Synthetic oligonucleotide”
gccuggaaagaaucaaaaccuuuga

SEQ ID NO: 106 - RNA, 25 nt, Artificial Sequence
  source: /note=“Description of Artificial Sequence: Synthetic oligonucleotide”
cugaacugaguuuaaaaggcaccca

SEQ ID NO: 107 - RNA, 25 nt, Artificial Sequence
  source: /note=“Description of Artificial Sequence: Synthetic oligonucleotide”
gaacugaguuuaaaaggcacccagc

SEQ ID NO: 108 - RNA, 25 nt, Artificial Sequence
  source: /note=“Description of Artificial Sequence: Synthetic oligonucleotide”
uaucagcgcagcuacugccauccaa

SEQ ID NO: 109 - RNA, 25 nt, Artificial Sequence
  source: /note=“Description of Artificial Sequence: Synthetic oligonucleotide”
ucaccaugcagauuaugcggaucaa

SEQ ID NO: 110 - RNA, 25 nt, Artificial Sequence
  source: /note=“Description of Artificial Sequence: Synthetic oligonucleotide”
agaugagcuuccuacagcacaacaa

SEQ ID NO: 111 - RNA, 25 nt, Artificial Sequence
  source: /note=“Description of Artificial Sequence: Synthetic oligonucleotide”
acaacaaaugugaaugcagaccaaa

SEQ ID NO: 112 - RNA, 25 nt, Artificial Sequence
  source: /note=“Description of Artificial Sequence: Synthetic oligonucleotide”
acaaaugugaaugcagaccaaagaa

SEQ ID NO: 113 - RNA, 25 nt, Artificial Sequence
  source: /note=“Description of Artificial Sequence: Synthetic oligonucleotide”
ggagcacuccaucacucuuaaucuu

SEQ ID NO: 114 - RNA, 25 nt, Artificial Sequence
  source: /note=“Description of Artificial Sequence: Synthetic oligonucleotide”
gguucaagcaucagcauuuggcauu

SEQ ID NO: 115 - RNA, 25 nt, Artificial Sequence
  source: /note=“Description of Artificial Sequence: Synthetic oligonucleotide”
gcauuuggcauuaagaaaucaccua

SEQ ID NO: 116 - RNA, 25 nt, Artificial Sequence
  source: /note=“Description of Artificial Sequence: Synthetic oligonucleotide”
gcaaauauggaaaucucuccaacua

SEQ ID NO: 117 - RNA, 25 nt, Artificial Sequence
  source: /note=“Description of Artificial Sequence: Synthetic oligonucleotide”
ccaagauuugcagaacuuguggaaa

SEQ ID NO: 118 - RNA, 25 nt, Artificial Sequence
  source: /note=“Description of Artificial Sequence: Synthetic oligonucleotide”
gguuuacauacucaacuccugccuu

SEQ ID NO: 119 - RNA, 25 nt, Artificial Sequence
  source: /note=“Description of Artificial Sequence: Synthetic oligonucleotide”
ggaaaguauuucagcuccgaaguuu

SEQ ID NO: 120 - RNA, 25 nt, Artificial Sequence
  source: /note=“Description of Artificial Sequence: Synthetic oligonucleotide”
ggaaacugacuuggccucggucauu

SEQ ID NO: 121 - RNA, 25 nt, Artificial Sequence
  source: /note=“Description of Artificial Sequence: Synthetic oligonucleotide”
ggccucggucauuuaugucuauguu

SEQ ID NO: 122 - RNA, 25 nt, Artificial Sequence
  source: /note=“Description of Artificial Sequence: Synthetic oligonucleotide”
gguucugaguccgucucauggaauu

SEQ ID NO: 123 - RNA, 25 nt, Artificial Sequence
  source: /note=“Description of Artificial Sequence: Synthetic oligonucleotide”
ggaccaaggagacuaugucugccuu

SEQ ID NO: 124 - RNA, 25 nt, Artificial Sequence
  source: /note=“Description of Artificial Sequence: Synthetic oligonucleotide”
cccuccacagaucaugugguuuaaa

SEQ ID NO: 125 - RNA, 25 nt, Artificial Sequence
  source: /note=“Description of Artificial Sequence: Synthetic oligonucleotide”
ggugauuguggaauucugcaaauuu

SEQ ID NO: 126 - RNA, 25 nt, Artificial Sequence
  source: /note=“Description of Artificial Sequence: Synthetic oligonucleotide”
ggaacauuugggaaaucucuugcaa

SEQ ID NO: 127 - skipped

SEQ ID NO: 128 - RNA, 25 nt, Artificial Sequence
  source: /note=“Description of Artificial Sequence: Synthetic oligonucleotide”
ccaaucgagacccugguggacaucu

SEQ ID NO: 129 - RNA, 25 nt, Artificial Sequence
  source: /note=“Description of Artificial Sequence: Synthetic oligonucleotide”
cccauaaauggucuuugccugaaau

SEQ ID NO: 130 - RNA, 25 nt, Artificial Sequence
  source: /note=“Description of Artificial Sequence: Synthetic oligonucleotide”
gagcauaacuaaaucugccugugga

SEQ ID NO: 131 - RNA, 25 nt, Artificial Sequence
  source: /note=“Description of Artificial Sequence: Synthetic oligonucleotide”
uggcaaacaauucugcaguacuuua

SEQ ID NO: 132 - RNA, 25 nt, Artificial Sequence
  source: /note=“Description of Artificial Sequence: Synthetic oligonucleotide”
ggcaaacaauucugcaguacuuuaa

SEQ ID NO: 133 - RNA, 25 nt, Artificial Sequence
  source: /note=“Description of Artificial Sequence: Synthetic oligonucleotide”
cagcucaagcaaaccacacuggcuu

SEQ ID NO: 134 - RNA, 25 nt, Artificial Sequence
  source: /note=“Description of Artificial Sequence: Synthetic oligonucleotide”
cagcugcaaauaucuagcuguaccu

SEQ ID NO: 135 - RNA, 25 nt, Artificial Sequence
  source: /note=“Description of Artificial Sequence: Synthetic oligonucleotide”
caaauaucuagcuguaccuacuuca

SEQ ID NO: 136 - RNA, 25 nt, Artificial Sequence
  source: /note=“Description of Artificial Sequence: Synthetic oligonucleotide”
gaaggaaacagaaucugcaaucuau

SEQ ID NO: 137 - RNA, 25 nt, Artificial Sequence
  source: /note=“Description of Artificial Sequence: Synthetic oligonucleotide”
cagguagaccuuucguagagaugua

SEQ ID NO: 138 - RNA, 25 nt, Artificial Sequence
  source: /note=“Description of Artificial Sequence: Synthetic oligonucleotide”
ugacugaaggaagggagcucgucau

SEQ ID NO: 139 - RNA, 25 nt, Artificial Sequence
  source: /note=“Description of Artificial Sequence: Synthetic oligonucleotide”
agaaauagggcuucugaccugugaa

SEQ ID NO: 140 - RNA, 25 nt, Artificial Sequence
  source: /note=“Description of Artificial Sequence: Synthetic oligonucleotide”
gaagcaacagucaaugggcauuugu

SEQ ID NO: 141 - RNA, 25 nt, Artificial Sequence
  source: /note=“Description of Artificial Sequence: Synthetic oligonucleotide”
gcaacagucaaugggcauuuguaua

SEQ ID NO: 142 - RNA, 25 nt, Artificial Sequence
  source: /note=“Description of Artificial Sequence: Synthetic oligonucleotide”
caacagucaaugggcauuuguauaa

SEQ ID NO: 143 - RNA, 25 nt, Artificial Sequence
  source: /note=“Description of Artificial Sequence: Synthetic oligonucleotide”
ccagucaaauuacuuagaggccaua

SEQ ID NO: 144 - RNA, 25 nt, Artificial Sequence
  source: /note=“Description of Artificial Sequence: Synthetic oligonucleotide”
ucaaauuacuuagaggccauacucu

SEQ ID NO: 145 - RNA, 25 nt, Artificial Sequence
  source: /note=“Description of Artificial Sequence: Synthetic oligonucleotide”
caaauuacuuagaggccauacucuu

SEQ ID NO: 146 - RNA, 25 nt, Artificial Sequence
  source: /note=“Description of Artificial Sequence: Synthetic oligonucleotide”
ggccauacucuuguccucaauugua

SEQ ID NO: 147 - RNA, 25 nt, Artificial Sequence
  source: /note=“Description of Artificial Sequence: Synthetic oligonucleotide”
uguccucaauuguacugcuaccacu

SEQ ID NO: 148 - RNA, 25 nt, Artificial Sequence
  source: /note=“Description of Artificial Sequence: Synthetic oligonucleotide”
ccacucccuugaacacgagaguuca

SEQ ID NO: 149 - RNA, 25 nt, Artificial Sequence
  source: /note=“Description of Artificial Sequence: Synthetic oligonucleotide”
gcggucuuaccggcucucuaugaaa

SEQ ID NO: 150 - RNA, 25 nt, Artificial Sequence
  source: /note=“Description of Artificial Sequence: Synthetic oligonucleotide”
ucccucgccggaaguuguaugguua

SEQ ID NO: 151 - RNA, 25 nt, Artificial Sequence
  source: /note=“Description of Artificial Sequence: Synthetic oligonucleotide”
ugcgacugagaaaucugcucgcuau

SEQ ID NO: 152 - RNA, 25 nt, Artificial Sequence
  source: /note=“Description of Artificial Sequence: Synthetic oligonucleotide”
uauuugacucguggcuacucguuaa

SEQ ID NO: 153 - RNA, 25 nt, Artificial Sequence
  source: /note=“Description of Artificial Sequence: Synthetic oligonucleotide”
ugacucguggcuacucguuaauuau

SEQ ID NO: 154 - RNA, 25 nt, Artificial Sequence
  source: /note=“Description of Artificial Sequence: Synthetic oligonucleotide”
gggaauuauacaaucuugcugagca

SEQ ID NO: 155 - RNA, 25 nt, Artificial Sequence
  source: /note=“Description of Artificial Sequence: Synthetic oligonucleotide”
cacugccacucuaauugucaaugug

SEQ ID NO: 156 - RNA, 25 nt, Artificial Sequence
  source: /note=“Description of Artificial Sequence: Synthetic oligonucleotide”
ggcagcagacaaauccugacuugua

SEQ ID NO: 157 - RNA, 25 nt, Artificial Sequence
  source: /note=“Description of Artificial Sequence: Synthetic oligonucleotide”
cagacaaauccugacuuguaccgca

SEQ ID NO: 158 - RNA, 25 nt, Artificial Sequence
  source: /note=“Description of Artificial Sequence: Synthetic oligonucleotide”
gacucuagaauuucuggaaucuaca

SEQ ID NO: 159 - RNA, 25 nt, Artificial Sequence
  source: /note=“Description of Artificial Sequence: Synthetic oligonucleotide”
uaucacagaugugccaaauggguuu

SEQ ID NO: 160 - RNA, 25 nt, Artificial Sequence
  source: /note=“Description of Artificial Sequence: Synthetic oligonucleotide”
gaugugccaaauggguuucauguua

SEQ ID NO: 161 - RNA, 25 nt, Artificial Sequence
  source: /note=“Description of Artificial Sequence: Synthetic oligonucleotide”
uggccaucacuaaggagcacuccau

SEQ ID NO: 162 - RNA, 25 nt, Artificial Sequence
  source: /note=“Description of Artificial Sequence: Synthetic oligonucleotide”
caucacuaaggagcacuccaucacu

SEQ ID NO: 163 - RNA, 25 nt, Artificial Sequence
  source: /note=“Description of Artificial Sequence: Synthetic oligonucleotide”
cacuaaggagcacuccaucacucuu

SEQ ID NO: 164 - RNA, 25 nt, Artificial Sequence
  source: /note=“Description of Artificial Sequence: Synthetic oligonucleotide”
ccaucacucuuaaucuuaccaucau

SEQ ID NO: 165 - RNA, 25 nt, Artificial Sequence
  source: /note=“Description of Artificial Sequence: Synthetic oligonucleotide”
caucacucuuaaucuuaccaucaug

SEQ ID NO: 166 - RNA, 25 nt, Artificial Sequence
  source: /note=“Description of Artificial Sequence: Synthetic oligonucleotide”
uaugccugcagagccaggaauguau

SEQ ID NO: 167 - RNA, 25 nt, Artificial Sequence
  source: /note=“Description of Artificial Sequence: Synthetic oligonucleotide”
aggaagcaccauaccuccugcgaaa

SEQ ID NO: 168 - RNA, 25 nt, Artificial Sequence
  source: /note=“Description of Artificial Sequence: Synthetic oligonucleotide”
ccagaagggcucuguggaaaguuca

SEQ ID NO: 169 - RNA, 25 nt, Artificial Sequence
  source: /note=“Description of Artificial Sequence: Synthetic oligonucleotide”
cagaagggcucuguggaaaguucag

SEQ ID NO: 170 - RNA, 25 nt, Artificial Sequence
  source: /note=“Description of Artificial Sequence: Synthetic oligonucleotide”
gggcucuguggaaaguucagcauac

SEQ ID NO: 171 - RNA, 25 nt, Artificial Sequence
  source: /note=“Description of Artificial Sequence: Synthetic oligonucleotide”
gaaccucggacaagucuaaucugga

SEQ ID NO: 172 - RNA, 25 nt, Artificial Sequence
  source: /note=“Description of Artificial Sequence: Synthetic oligonucleotide”
gcugaucacucuaacaugcaccugu

SEQ ID NO: 173 - RNA, 25 nt, Artificial Sequence
  source: /note=“Description of Artificial Sequence: Synthetic oligonucleotide”
gcgacucucuucuggcuccuauuaa

SEQ ID NO: 174 - RNA, 25 nt, Artificial Sequence
  source: /note=“Description of Artificial Sequence: Synthetic oligonucleotide”
ccuaucaauuauaauggacccagau

SEQ ID NO: 175 - RNA, 25 nt, Artificial Sequence
  source: /note=“Description of Artificial Sequence: Synthetic oligonucleotide”
aagcaucagcauuuggcauuaagaa

SEQ ID NO: 176 - RNA, 25 nt, Artificial Sequence
  source: /note=“Description of Artificial Sequence: Synthetic oligonucleotide”
gccaccaucugaacgugguuaaccu

SEQ ID NO: 177 - RNA, 25 nt, Artificial Sequence
  source: /note=“Description of Artificial Sequence: Synthetic oligonucleotide”
ggccucugauggugauuguugaaua

SEQ ID NO: 178 - RNA, 25 nt, Artificial Sequence
  source: /note=“Description of Artificial Sequence: Synthetic oligonucleotide”
ccucugauggugauuguugaauacu

SEQ ID NO: 179 - RNA, 25 nt, Artificial Sequence
  source: /note=“Description of Artificial Sequence: Synthetic oligonucleotide”
gauggugauuguugaauacugcaaa

SEQ ID NO: 180 - RNA, 25 nt, Artificial Sequence
  source: /note=“Description of Artificial Sequence: Synthetic oligonucleotide”
gagcuccggcuuucaggaagauaaa

SEQ ID NO: 181 - RNA, 25 nt, Artificial Sequence
  source: /note=“Description of Artificial Sequence: Synthetic oligonucleotide”
cauggaguuccugucuuccagaaag

SEQ ID NO: 182 - RNA, 25 nt, Artificial Sequence
  source: /note=“Description of Artificial Sequence: Synthetic oligonucleotide”
gaguuccugucuuccagaaagugca

SEQ ID NO: 183 - RNA, 25 nt, Artificial Sequence
  source: /note=“Description of Artificial Sequence: Synthetic oligonucleotide”
gcaugaggaugagagcuccugagua

SEQ ID NO: 184 - RNA, 25 nt, Artificial Sequence
  source: /note=“Description of Artificial Sequence: Synthetic oligonucleotide”
gagagcuccugaguacucuacuccu

SEQ ID NO: 185 - RNA, 25 nt, Artificial Sequence
  source: /note=“Description of Artificial Sequence: Synthetic oligonucleotide”
ggccaagauuugcagaacuugugga

SEQ ID NO: 186 - RNA, 25 nt, Artificial Sequence
  source: /note=“Description of Artificial Sequence: Synthetic oligonucleotide”
aacuaggugauuugcuucaagcaaa

SEQ ID NO: 187 - RNA, 25 nt, Artificial Sequence
  source: /note=“Description of Artificial Sequence: Synthetic oligonucleotide”
aggugauuugcuucaagcaaaugua

SEQ ID NO: 188 - RNA, 25 nt, Artificial Sequence
  source: /note=“Description of Artificial Sequence: Synthetic oligonucleotide”
ugacaggaaauaguggguuuacaua

SEQ ID NO: 189 - RNA, 25 nt, Artificial Sequence
  source: /note=“Description of Artificial Sequence: Synthetic oligonucleotide”
ggaaauaguggguuuacauacucaa

SEQ ID NO: 190 - RNA, 25 nt, Artificial Sequence
  source: /note=“Description of Artificial Sequence: Synthetic oligonucleotide”
ggccucgcucaagauugacuugaga

SEQ ID NO: 191 - RNA, 25 nt, Artificial Sequence
  source: /note=“Description of Artificial Sequence: Synthetic oligonucleotide”
ucgcucaagauugacuugagaguaa

SEQ ID NO: 192 - RNA, 25 nt, Artificial Sequence
  source: /note=“Description of Artificial Sequence: Synthetic oligonucleotide”
gauugacuugagaguaaccaguaaa

SEQ ID NO: 193 - RNA, 25 nt, Artificial Sequence
  source: /note=“Description of Artificial Sequence: Synthetic oligonucleotide”
cagacuacaacucggugguccugua

SEQ ID NO: 194 - RNA, 25 nt, Artificial Sequence
  source: /note=“Description of Artificial Sequence: Synthetic oligonucleotide”
gacuacaacucggugguccuguacu

SEQ ID NO: 195 - RNA, 25 nt, Artificial Sequence
  source: /note=“Description of Artificial Sequence: Synthetic oligonucleotide”
cagcacgcuguuuauugaaagaguc

SEQ ID NO: 196 - RNA, 25 nt, Artificial Sequence
  source: /note=“Description of Artificial Sequence: Synthetic oligonucleotide”
gcacgcuguuuauugaaagagucac

SEQ ID NO: 197 - DNA, 4017 bp, Homo sapiens
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctctatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctctttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcctgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatatgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctag

SEQ ID NO: 198 - DNA, 2651 bp, Homo sapiens
gcggacactcctctcggctcctccccggcagcggcggcggctcggagcgggctccggggctcgggtgcagcggccagcgggcctggcggcgaggattacccggggaagtggttgtctcctggctggagccgcgagacgggcgctcagggcgcggggccggcggcggcgaacgagaggacggactctggcggccgggtcgttggccgggggagcgcgggcaccgggcgagcaggccgcgtcgcgctcaccatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagaggtgagcactgcaacaaaaaggctgttttctctcggatctccaaatttaaaagcacaaggaatgattgtaccacacaaagtaatgtaaaacattaaaggactcattaaaaagtaacagttgtctcatatcatcttgatttattgtcactgttgctaactttcaggctcggaggagatgctcctcccaaaatgagttcggagatgatagcagtaataatgagacccccgggctccagctctgggccccccattcaggccgagggggctgctccggggggccgacttggtgcacgtttggatttggaggatccctgcactgccttctctgtgtttgttgctcttgctgttttctcctgcctgataaacaacaacttgggatgatcctttccattttgatgccaacctctttttatttttaagcggcgccctatagt

SEQ ID NO: 199 - DNA, 6280 bp, Mus musculus
agcgcggtggcggacactcccgggaggtagtgctagtggtggtggctgctgctcggagcgggctccgggactcaagcgcagcggctagcggacgcgggacggcgaggatccccccacaccacccccctcggctgcaggcgcggagaagggctctcgcggcgccaagcagaagcaggaggggaccggctcgagcggctgcgccgtcggcctcggagagcgcgggcaccgggccaacaggccgcgtcttgctcaccatggtcagctgctgggacaccgcggtcttgccttacgcgctgctcgggtgtctgcttctcacaggatatggctcagggtcgaagttaaaagtgcctgaactgagtttaaaaggcacccagcatgtcatgcaagcaggccagactctctttctcaagtgcagaggggaggcagcccactcatggtctctgcccacgaccgtgagccaggaggacaaaaggctgagcatcactcccccatcggcctgtgggagggataacaggcaattctgcagcaccttgaccttggacacggcgcaggccaaccacacgggcctctacacctgtagatacctccctacatctacttcgaagaaaaagaaagcggaatcttcaatctacatatttgttagtgatgcagggagtcctttcatagagatgcacactgacatacccaaacttgtgcacatgacggaaggaagacagctcatcatcccctgccgggtgacgtcacccaacgtcacagtcaccctaaaaaagtttccatttgatactcttacccctgatgggcaaagaataacatgggacagtaggagaggctttataatagcaaatgcaacgtacaaagagataggactgctgaactgcgaagccaccgtcaacgggcacctgtaccagacaaactatctgacccatcggcagaccaatacaatcctagatgtccaaatacgcccgccgagcccagtgagactgctccacgggcagactcttgtcctcaactgcaccgccaccacggagctcaatacgagggtgcaaatgagctggaattaccctggtaaagcaactaagagagcatctataaggcagcggattgaccggagccattcccacaacaatgtgttccacagtgttcttaagatcaacaatgtggagagccgagacaaggggctctacacctgtcgcgtgaagagtgggtcctcgttccagtctttcaacacctccgtgcatgtgtatgaaaaaggattcatcagtgtgaaacatcggaagcagccggtgcaggaaaccacagcaggaagacggtcctatcggctgtccatgaaagtgaaggccttcccctccccagaaatcgtatggttaaaagatggctcgcctgcaacattgaagtctgctcgctatttggtacatggctactcattaattatcaaagatgtgacaaccgaggatgcaggggactatacgatcttgctgggcataaagcagtcaaggctatttaaaaacctcactgccactctcattgtaaacgtgaaacctcagatctacgaaaagtccgtgtcctcgcttccaagcccacctctctatccgctgggcagcagacaagtcctcacttgcaccgtgtatggcatccctcggccaacaatcacgtggctctggcacccctgtcaccacaatcactccaaagaaaggtatgacttctgcactgagaatgaagaatcctttatcctggatcccagcagcaacttaggaaacagaattgagagcatctctcagcgcatgacggtcatagaaggaacaaataagacggttagcacattggtggtggctgactctcagacccctggaatctacagctgccgggccttcaataaaatagggactgtggaaagaaacataaaattttatgtcacagatgtgccgaatggctttcacgtttccttggaaaagatgccagccgaaggagaggacctgaaactgtcctgtgtggtcaataaattcctgtacagagacattacctggattctgctacggacagttaacaacagaaccatgcaccatagtatcagcaagcaaaaaatggccaccactcaagattactccatcactctgaaccttgtcatcaagaacgtgtctctagaagactcgggcacctatgcgtgcagagccaggaacatatacacaggggaagacatccttcggaagacagaagttctcgttagagattcggaagcgccacacctgcttcaaaacctcagtgactacgaggtctccatcagtggctctacgaccttagactgtcaagctagaggtgtccccgcgcctcagatcacttggttcaaaaacaaccacaaaatacaacaagaaccgggaattattttaggaccaggaaacagcacgctgtttattgaaagagtcacagaggaggatgagggtgtctataggtgccgagccaccaaccagaagggggccgtggaaagcgcagcctacctcaccgtgcaaggaacctcagacaagtcaaacctggagctgatcacgctcacgtgcacatgcgtggctgcgaccctcttttggctccttctaactctcttcatcagaaaactgaagcggtcttcttccgaagtaaagacagactacctgtcaatcattatggacccagatgaagttcccctggatgagcagtgtgaacggctgccctatgatgccagcaagtgggagtttgcacgggagagactgaaactaggcaaatcgctcggaagaggggcttttgggaaagtcgttcaagcctctgcatttggcattaagaaatcacccacctgccggactgtggctgtgaagatgttgaaagagggggccacagccagtgagtacaaagctctgatgaccgaactcaagatcttgacccacatcggccatcatctgaatgtggttaacctcctgggagcctgcacgaagcaaggagggcctctgatggtgatcgtggaatactgcaaatacggaaacctgtccaactacctcaagagcaaacgtgacttattctgtctcaacaaggacgcagccttgcatatggagctcaagaaagagagcctggaaccaggcctggagcagggccagaagccccgcctagacagtgtcagcagctcaagtgtcaccagctccagcttccctgaagaccgaagcgtgagcgatgtggaaggagacgaggattacagtgagatctccaagcagcccctcaccatggaagacctgatttcctacagtttccaagtggccagaggcatggagtttctgtcctccagaaagtgcattcatcgggacctggcagcgagaaacatccttttatctgagaacaatgtggtgaagatttgcgactttggcctggcccgggatatttataagaaccctgattatgtgaggagaggagatactcgacttcccctaaaatggatggctcctgaatccatctttgacaaggtctacagcaccaagagcgatgtgtggtcctatggcgtgttgctgtgggagatcttctccttagggggttctccatacccaggagtgcaaatggatgaagacttctgcagccgcctgaaggaaggcatgcggatgagaaccccggagtatgccacacctgaaatctaccaaatcatgttggattgctggcacaaagaccccaaagagaggccccggtttgctgaacttgtggagaaacttggtgacctgcttcaagccaacgtccaacaggatgggaaagattacatccccctcaatgccatactgactagaaacagtggcttcacatactcgacccccaccttctctgaggaccttttcaaggacggctttgcagatccacattttcattccggaagctctgatgatgtgagatatgtaaacgctttcaaattcatgagcctggaaagaatcaaaacctttgaggagctttcaccgaactccacctccatgtttgaggactatcagctggacactagcactctgctgggctcccccttgctgaagcggttcacctggactgagaccaagcccaaggcctccatgaagatagacttgagaatagcgagtaaaagcaaggaggcgggactttccgatctgccgaggcccagcttctgcttctccagctgtggccacatcaggcccgtgcaggacgatgaatctgagctgggaaaggagtcctgctgttctccacccccagactacaactccgtggtgttgtactcctccccgcccgcctaaagcttctcaccagccccgacaaccagcccctgacagtattatacatctatgagtttacacctattccgctccacaggagccagctgcttttcgtgacctttaatcgtgcttttttgttttttgttttgtttgttgttgctgttttgactaacaagaatgtaaccccagttagtgacgtgtgaagaatactattgttagagaaatcccccccgcaaagcctcagggtaacctggacaggaaggagcaggtgcctctggcgaccgccccgcccaccggccatggccccacccaccctccctgcagctgtgggactagaggcagtaagcccattagctcatggctgcatgcactgacctgctctgtctctcttatggaggaaagggagaacagagcaaacaggaggcacaggaaaaggctttgggatgcgtccgtcctgtggagcccgtgcaggagggggctccgctatgccacttcagtgacttctcactcctggcctccgctgtttcgggcccccttccaagaggtatcagagcagaacatgagggacgtttcctagaccagggcacatgttctcgggaaccacagttaatcttaaatcttttcccgggagtcttctgttgtctgtttaccatccaaagcatatttaacatgtgtcagtgggggtggcgcttggcttctgaggccagagccatcatcagttcctctagtgagatgcattgaggtcatacccaagcttgcaggcctgaccttcgcatactgctcacggggagttaagtggtccagtttggcctagtaaggttgcctactgatgggctcaaaagccacattttaaacaggttttatctcaagtattaatatatagacaagacacttatgcattatcctgttttatatatccaatgaatataactggggcgagttaagagtcatggtctagaaaaggggtttctctgtacccaaatcgggctggttggaccaagacccagagaggcagagtggttgtcccagctatagttactaaactactcacccaaagttgggacctcactggcttctctttacttcatcatggatttcaccatcccaaggcagtctgagaggagctaaagagtatcagcccatatttattaagcactttatgctccttggcacagcaggtgatgtgtaatttatgcaagctccctctccagctaggactcaggatattagtcaatgagccatcaaaaggaaaaaaaaaaaaccatcttattttcatctgtttcataccttgtctggggtctaatgacgatggcaacagggtagacatgggaagacagggtagaaaagggtgcccgctctttggggtctagagatgagccctgggtctctaaaatggctctcttagaagttgtatgtgcaaattatggtctgtgtgcttaggtcgtgcacacctgccggagccggtcacagctgggcagacgatgaatagctgctttgggagagcagagcatgctagccacttaattctctgaccgggccagcatcatgggtacctgctcccctgtgtaccccatccttaaggttttctgtctgatgagactggaggcccagtgcaatccccactgagacagcctgcagcccactgtggctcttggtgcactcaccagccaggactagacaagtaggaaagggcttctagccacactggagaaaaagaaaatcaggtagggctggccaaagacatctttgtccattcgcaaaagctcttgtcggctgcagtgtgtaagtcaggcgatgagacagaggctaccagagaaacggatgagaacagcagcctgaggtttctcatccagatatccagcaattggggggtgggggaagaccatagatggtcctgtattattccgattttaataatctaattcgtgatcattaagagactttagtaaatgtccctttcccacaaaagtaaagaaaagctatcgggattctctggttctgcttaaagacttagctttggagcctatgaaagttgatcagccagg

SEQ ID NO: 200 - protein, 4 aa, Artificial Sequence
  source: /note=“Description of Artificial Sequence: Synthetic peptide”
KHHH

SEQ ID NO: 201 - protein, 18 aa, Artificial Sequence
  source: /note=“Description of Artificial Sequence: Synthetic peptide”
KHHHKHHHKHHHHKHHHK

SEQ ID NO: 202 - protein, 19 aa, Artificial Sequence
  source: /note=“Description of Artificial Sequence: Synthetic peptide”
CKHHHKHHHKHHHHKHHHK

SEQ ID NO: 203 - protein, 5 aa, Artificial Sequence
  source: /note=“Description of Artificial Sequence: Synthetic peptide”
RGDFK

SEQ ID NO: 204 - protein, 5 aa, Artificial Sequence
  source: /note=“Description of Artificial Sequence: Synthetic peptide”
  MOD_RES (5)..(5): D-Phe
RGDFK

SEQ ID NO: 205 - protein, 17 aa, Artificial Sequence
  source: /note=“Description of Artificial Sequence: Synthetic peptide”
KHHHKHHHKHHHKHHHK

SEQ ID NO: 206 - RNA, 25 nt, Artificial Sequence
  source: /note=“Description of Artificial Sequence: Synthetic oligonucleotide”
auuucaggcaaagaccauuuauggg

SEQ ID NO: 207 - RNA, 25 nt, Artificial Sequence
  source: /note=“Description of Artificial Sequence: Synthetic oligonucleotide”
uccacaggcagauuuaguuaugcuc

SEQ ID NO: 208 - RNA, 25 nt, Artificial Sequence
  source: /note=“Description of Artificial Sequence: Synthetic oligonucleotide”
uaaaguacugcagaauuguuugcca

SEQ ID NO: 209 - RNA, 25 nt, Artificial Sequence
  source: /note=“Description of Artificial Sequence: Synthetic oligonucleotide”
uaaaguacugcagaauuguuugcca

SEQ ID NO: 210 - RNA, 25 nt, Artificial Sequence
  source: /note=“Description of Artificial Sequence: Synthetic oligonucleotide”
aagccagugugguuugcuugagcug

SEQ ID NO: 211 - RNA, 25 nt, Artificial Sequence
  source: /note=“Description of Artificial Sequence: Synthetic oligonucleotide”
agguacagcuagauauuugcagcug

SEQ ID NO: 212 - RNA, 25 nt, Artificial Sequence
  source: /note=“Description of Artificial Sequence: Synthetic oligonucleotide”
ugaaguagguacagcuagauauuug

SEQ ID NO: 213 - RNA, 25 nt, Artificial Sequence
  source: /note=“Description of Artificial Sequence: Synthetic oligonucleotide”
auagauugcagauucuguuuccuuc

SEQ ID NO: 214 - RNA, 25 nt, Artificial Sequence
  source: /note=“Description of Artificial Sequence: Synthetic oligonucleotide”
uacaucucuacgaaaggucuaccug

SEQ ID NO: 215 - RNA, 25 nt, Artificial Sequence
  source: /note=“Description of Artificial Sequence: Synthetic oligonucleotide”
augacgagcucccuuccuucaguca

SEQ ID NO: 216 - RNA, 25 nt, Artificial Sequence
  source: /note=“Description of Artificial Sequence: Synthetic oligonucleotide”
uucacaggucagaagcccuauuucu

SEQ ID NO: 217 - RNA, 25 nt, Artificial Sequence
  source: /note=“Description of Artificial Sequence: Synthetic oligonucleotide”
acaaaugcccauugacuguugcuuc

SEQ ID NO: 218 - RNA, 25 nt, Artificial Sequence
  source: /note=“Description of Artificial Sequence: Synthetic oligonucleotide”
uauacaaaugcccauugacuguugc

SEQ ID NO: 219 - RNA, 25 nt, Artificial Sequence
  source: /note=“Description of Artificial Sequence: Synthetic oligonucleotide”
uauacaaaugcccauugacuguugc

SEQ ID NO: 220 - RNA, 25 nt, Artificial Sequence
  source: /note=“Description of Artificial Sequence: Synthetic oligonucleotide”
uauggccucuaaguaauuugacugg

SEQ ID NO: 221 - RNA, 25 nt, Artificial Sequence
  source: /note=“Description of Artificial Sequence: Synthetic oligonucleotide”
agaguauggccucuaaguaauuuga

SEQ ID NO: 222 - RNA, 25 nt, Artificial Sequence
  source: /note=“Description of Artificial Sequence: Synthetic oligonucleotide”
aagaguauggccucuaaguaauuug

SEQ ID NO: 223 - RNA, 25 nt, Artificial Sequence
  source: /note=“Description of Artificial Sequence: Synthetic oligonucleotide”
uacaauugaggacaagaguauggcc

SEQ ID NO: 224 - RNA, 25 nt, Artificial Sequence
  source: /note=“Description of Artificial Sequence: Synthetic oligonucleotide”
agugguagcaguacaauugaggaca

SEQ ID NO: 225 - RNA, 25 nt, Artificial Sequence
  source: /note=“Description of Artificial Sequence: Synthetic oligonucleotide”
ugaacucucguguucaagggagugg

SEQ ID NO: 226 - RNA, 25 nt, Artificial Sequence
  source: /note=“Description of Artificial Sequence: Synthetic oligonucleotide”
uuucauagagagccgguaagaccgc

SEQ ID NO: 227 - RNA, 25 nt, Artificial Sequence
  source: /note=“Description of Artificial Sequence: Synthetic oligonucleotide”
uaaccauacaacuuccggcgaggga

SEQ ID NO: 228 - RNA, 25 nt, Artificial Sequence
  source: /note=“Description of Artificial Sequence: Synthetic oligonucleotide”
auagcgagcagauuucucagucgca

SEQ ID NO: 229 - RNA, 25 nt, Artificial Sequence
  source: /note=“Description of Artificial Sequence: Synthetic oligonucleotide”
uuaacgaguagccacgagucaaaua

SEQ ID NO: 230 - RNA, 25 nt, Artificial Sequence
  source: /note=“Description of Artificial Sequence: Synthetic oligonucleotide”
auaauuaacgaguagccacgaguca

SEQ ID NO: 231 - RNA, 25 nt, Artificial Sequence
  source: /note=“Description of Artificial Sequence: Synthetic oligonucleotide”
ugcucagcaagauuguauaauuccc

SEQ ID NO: 232 - RNA, 25 nt, Artificial Sequence
  source: /note=“Description of Artificial Sequence: Synthetic oligonucleotide”
cacauugacaauuagaguggcagug

SEQ ID NO: 233 - RNA, 25 nt, Artificial Sequence
  source: /note=“Description of Artificial Sequence: Synthetic oligonucleotide”
uacaagucaggauuugucugcugcc

SEQ ID NO: 234 - RNA, 25 nt, Artificial Sequence
  source: /note=“Description of Artificial Sequence: Synthetic oligonucleotide”
ugcgguacaagucaggauuugucug

SEQ ID NO: 235 - RNA, 25 nt, Artificial Sequence
  source: /note=“Description of Artificial Sequence: Synthetic oligonucleotide”
uguagauuccagaaauucuagaguc

SEQ ID NO: 236 - RNA, 25 nt, Artificial Sequence
  source: /note=“Description of Artificial Sequence: Synthetic oligonucleotide”
aaacccauuuggcacaucugugaua

SEQ ID NO: 237 - RNA, 25 nt, Artificial Sequence
  source: /note=“Description of Artificial Sequence: Synthetic oligonucleotide”
uaacaugaaacccauuuggcacauc

SEQ ID NO: 238 - RNA, 25 nt, Artificial Sequence
  source: /note=“Description of Artificial Sequence: Synthetic oligonucleotide”
auggagugcuccuuagugauggcca

SEQ ID NO: 239 - RNA, 25 nt, Artificial Sequence
  source: /note=“Description of Artificial Sequence: Synthetic oligonucleotide”
agugauggagugcuccuuagugaug

SEQ ID NO: 240 - RNA, 25 nt, Artificial Sequence
  source: /note=“Description of Artificial Sequence: Synthetic oligonucleotide”
aagagugauggagugcuccuuagug

SEQ ID NO: 241 - RNA, 25 nt, Artificial Sequence
  source: /note=“Description of Artificial Sequence: Synthetic oligonucleotide”
augaugguaagauuaagagugaugg

SEQ ID NO: 242 - RNA, 25 nt, Artificial Sequence
  source: /note=“Description of Artificial Sequence: Synthetic oligonucleotide”
augaugguaagauuaagagugaugg

SEQ ID NO: 243 - RNA, 25 nt, Artificial Sequence
  source: /note=“Description of Artificial Sequence: Synthetic oligonucleotide”
auacauuccuggcucugcaggcaua

SEQ ID NO: 244 - RNA, 25 nt, Artificial Sequence
  source: /note=“Description of Artificial Sequence: Synthetic oligonucleotide”
uuucgcaggagguauggugcuuccu

SEQ ID NO: 245 - RNA, 25 nt, Artificial Sequence
  source: /note=“Description of Artificial Sequence: Synthetic oligonucleotide”
uucugugacucuuucaauaaacagc

SEQ ID NO: 246 - RNA, 25 nt, Artificial Sequence
  source: /note=“Description of Artificial Sequence: Synthetic oligonucleotide”
ugaacuuuccacagagcccuucugg

SEQ ID NO: 247 - RNA, 25 nt, Artificial Sequence
  source: /note=“Description of Artificial Sequence: Synthetic oligonucleotide”
cugaacuuuccacagagcccuucug

SEQ ID NO: 248 - RNA, 25 nt, Artificial Sequence
  source: /note=“Description of Artificial Sequence: Synthetic oligonucleotide”
guaugcugaacuuuccacagagccc

SEQ ID NO: 249 - RNA, 25 nt, Artificial Sequence
  source: /note=“Description of Artificial Sequence: Synthetic oligonucleotide”
uccagauuagacuuguccgagguuc

SEQ ID NO: 250 - RNA, 25 nt, Artificial Sequence
  source: /note=“Description of Artificial Sequence: Synthetic oligonucleotide”
acaggugcauguuagagugaucagc

SEQ ID NO: 251 - RNA, 25 nt, Artificial Sequence
  source: /note=“Description of Artificial Sequence: Synthetic oligonucleotide”
uuaauaggagccagaagagagucgc

SEQ ID NO: 252 - RNA, 25 nt, Artificial Sequence
  source: /note=“Description of Artificial Sequence: Synthetic oligonucleotide”
aucuggguccauuauaauugauagg

SEQ ID NO: 253 - RNA, 25 nt, Artificial Sequence
  source: /note=“Description of Artificial Sequence: Synthetic oligonucleotide”
uucuuaaugccaaaugcugaugcuu

SEQ ID NO: 254 - RNA, 25 nt, Artificial Sequence
  source: /note=“Description of Artificial Sequence: Synthetic oligonucleotide”
agguuaaccacguucagaugguggc

SEQ ID NO: 255 - RNA, 25 nt, Artificial Sequence
  source: /note=“Description of Artificial Sequence: Synthetic oligonucleotide”
uauucaacaaucaccaucagaggcc

SEQ ID NO: 256 - RNA, 25 nt, Artificial Sequence
  source: /note=“Description of Artificial Sequence: Synthetic oligonucleotide”
aguauucaacaaucaccaucagagg

SEQ ID NO: 257 - RNA, 25 nt, Artificial Sequence
  source: /note=“Description of Artificial Sequence: Synthetic oligonucleotide”
uuugcaguauucaacaaucaccauc

SEQ ID NO: 258 - RNA, 25 nt, Artificial Sequence
  source: /note=“Description of Artificial Sequence: Synthetic oligonucleotide”
aauaagucacguuugcucuugaggu

SEQ ID NO: 259 - RNA, 25 nt, Artificial Sequence
  source: /note=“Description of Artificial Sequence: Synthetic oligonucleotide”
aaauaagucacguuugcucuugagg

SEQ ID NO: 260 - RNA, 25 nt, Artificial Sequence
  source: /note=“Description of Artificial Sequence: Synthetic oligonucleotide”
uuuaucuuccugaaagccggagcuc

SEQ ID NO: 261 - RNA, 25 nt, Artificial Sequence
  source: /note=“Description of Artificial Sequence: Synthetic oligonucleotide”
cuuucuggaagacaggaacuccaug

SEQ ID NO: 262 - RNA, 25 nt, Artificial Sequence
  source: /note=“Description of Artificial Sequence: Synthetic oligonucleotide”
ugcacuuucuggaagacaggaacuc

SEQ ID NO: 263 - RNA, 25 nt, Artificial Sequence
  source: /note=“Description of Artificial Sequence: Synthetic oligonucleotide”
uacucaggagcucucauccucaugc

SEQ ID NO: 264 - RNA, 25 nt, Artificial Sequence
  source: /note=“Description of Artificial Sequence: Synthetic oligonucleotide”
aggaguagaguacucaggagcucuc

SEQ ID NO: 265 - RNA, 25 nt, Artificial Sequence
  source: /note=“Description of Artificial Sequence: Synthetic oligonucleotide”
uccacaaguucugcaaaucuuggcc

SEQ ID NO: 266 - RNA, 25 nt, Artificial Sequence
  source: /note=“Description of Artificial Sequence: Synthetic oligonucleotide”
uuugcuugaagcaaaucaccuaguu

SEQ ID NO: 267 - RNA, 25 nt, Artificial Sequence
  source: /note=“Description of Artificial Sequence: Synthetic oligonucleotide”
uacauuugcuugaagcaaaucaccu

SEQ ID NO: 268 - RNA, 25 nt, Artificial Sequence
  source: /note=“Description of Artificial Sequence: Synthetic oligonucleotide”
uauguaaacccacuauuuccuguca

SEQ ID NO: 269 - RNA, 25 nt, Artificial Sequence
  source: /note=“Description of Artificial Sequence: Synthetic oligonucleotide”
uugaguauguaaacccacuauuucc

SEQ ID NO: 270 - RNA, 25 nt, Artificial Sequence
  source: /note=“Description of Artificial Sequence: Synthetic oligonucleotide”
aaacuucggagcugaaauacuuucc

SEQ ID NO: 271 - RNA, 25 nt, Artificial Sequence
  source: /note=“Description of Artificial Sequence: Synthetic oligonucleotide”
ucucaagucaaucuugagcgaggcc

SEQ ID NO: 272 - RNA, 25 nt, Artificial Sequence
  source: /note=“Description of Artificial Sequence: Synthetic oligonucleotide”
uuacucucaagucaaucuugagcga

SEQ ID NO: 273 - RNA, 25 nt, Artificial Sequence
  source: /note=“Description of Artificial Sequence: Synthetic oligonucleotide”
uuuacugguuacucucaagucaauc

SEQ ID NO: 274 - RNA, 25 nt, Artificial Sequence
  source: /note=“Description of Artificial Sequence: Synthetic oligonucleotide”
uacaggaccaccgaguuguagucug

SEQ ID NO: 275 - RNA, 25 nt, Artificial Sequence
  source: /note=“Description of Artificial Sequence: Synthetic oligonucleotide”
aguacaggaccaccgaguuguaguc

SEQ ID NO: 276 - RNA, 25 nt, Artificial Sequence
  source: /note=“Description of Artificial Sequence: Synthetic oligonucleotide”
ugggugccuuuuaaacucaguucag

SEQ ID NO: 277 - RNA, 25 nt, Artificial Sequence
  source: /note=“Description of Artificial Sequence: Synthetic oligonucleotide”
gcugggugccuuuuaaacucaguuc

SEQ ID NO: 278 - RNA, 25 nt, Artificial Sequence
  source: /note=“Description of Artificial Sequence: Synthetic oligonucleotide”
gacucuuucaauaaacagcgugcug

SEQ ID NO: 279 - RNA, 25 nt, Artificial Sequence
  source: /note=“Description of Artificial Sequence: Synthetic oligonucleotide”
gugacucuuucaauaaacagcgugc

SEQ ID NO: 280 - RNA, 25 nt, Artificial Sequence
  source: /note=“Description of Artificial Sequence: Synthetic oligonucleotide”
ugugacucuuucaauaaacagcgug

SEQ ID NO: 281 - RNA, 25 nt, Artificial Sequence
  source: /note=“Description of Artificial Sequence: Synthetic oligonucleotide”
ucugugacucuuucaauaaacagcg

SEQ ID NO: 282 - RNA, 25 nt, Artificial Sequence
  source: /note=“Description of Artificial Sequence: Synthetic oligonucleotide”
aauaagucacguuugcucuugaggu

SEQ ID NO: 283 - RNA, 25 nt, Artificial Sequence
  source: /note=“Description of Artificial Sequence: Synthetic oligonucleotide”
uaagucacguuugcucuugagguag

SEQ ID NO: 284 - RNA, 25 nt, Artificial Sequence
  source: /note=“Description of Artificial Sequence: Synthetic oligonucleotide”
aauaagucacguuugcucuugaggu

SEQ ID NO: 285 - RNA, 25 nt, Artificial Sequence
  source: /note=“Description of Artificial Sequence: Synthetic oligonucleotide”
aaagguuuugauucuuuccaggcuc

SEQ ID NO: 286 - RNA, 25 nt, Artificial Sequence
  source: /note=“Description of Artificial Sequence: Synthetic oligonucleotide”
ucaaagguuuugauucuuuccaggc

SEQ ID NO: 287 - RNA, 25 nt, Artificial Sequence
  source: /note=“Description of Artificial Sequence: Synthetic oligonucleotide”
uuaaccauacaacuuccggcgaggg

SEQ ID NO: 288 - RNA, 25 nt, Artificial Sequence
  source: /note=“Description of Artificial Sequence: Synthetic oligonucleotide”
uuagcaugacagucuaaaguggugg

SEQ ID NO: 289 - RNA, 25 nt, Artificial Sequence
  source: /note=“Description of Artificial Sequence: Synthetic oligonucleotide”
aucagcuccagauuagacuuguccg

SEQ ID NO: 290 - RNA, 25 nt, Artificial Sequence
  source: /note=“Description of Artificial Sequence: Synthetic oligonucleotide”
uucagaugguggccaauguggguca

SEQ ID NO: 291 - RNA, 25 nt, Artificial Sequence
  source: /note=“Description of Artificial Sequence: Synthetic oligonucleotide”
ucaacaaucaccaucagaggcccuc

SEQ ID NO: 292 - RNA, 25 nt, Artificial Sequence
  source: /note=“Description of Artificial Sequence: Synthetic oligonucleotide”
uuaucuuccugaaagccggagcucg

SEQ ID NO: 293 - RNA, 25 nt, Artificial Sequence
  source: /note=“Description of Artificial Sequence: Synthetic oligonucleotide”
uuuccugucaguauggcauugauug

SEQ ID NO: 294 - RNA, 25 nt, Artificial Sequence
  source: /note=“Description of Artificial Sequence: Synthetic oligonucleotide”
uaaacuucggagcugaaauacuuuc

SEQ ID NO: 295 - RNA, 25 nt, Artificial Sequence
  source: /note=“Description of Artificial Sequence: Synthetic oligonucleotide”
ugaagauguacucgaucucaucagg

SEQ ID NO: 296 - RNA, 25 nt, Artificial Sequence
  source: /note=“Description of Artificial Sequence: Synthetic oligonucleotide”
uugugcuguaggaagcucaucucuc

SEQ ID NO: 297 - RNA, 25 nt, Artificial Sequence
  source: /note=“Description of Artificial Sequence: Synthetic oligonucleotide”
uuggucugcauucacauuuguugug

SEQ ID NO: 298 - RNA, 25 nt, Artificial Sequence
  source: /note=“Description of Artificial Sequence: Synthetic oligonucleotide”
uaagaacacuguagaauauguuggc

SEQ ID NO: 299 - RNA, 25 nt, Artificial Sequence
  source: /note=“Description of Artificial Sequence: Synthetic oligonucleotide”
aauugugagugucuuacagaagagg

SEQ ID NO: 300 - RNA, 25 nt, Artificial Sequence
  source: /note=“Description of Artificial Sequence: Synthetic oligonucleotide”
aacauuuacacgucugcggaucuug

SEQ ID NO: 301 - RNA, 25 nt, Artificial Sequence
  source: /note=“Description of Artificial Sequence: Synthetic oligonucleotide”
aguacguucguuuaacucaagcugc

SEQ ID NO: 302 - RNA, 25 nt, Artificial Sequence
  source: /note=“Description of Artificial Sequence: Synthetic oligonucleotide”
aaguacguucguuuaacucaagcug

SEQ ID NO: 303 - DNA, 25 bp, Artificial Sequence
  source: /note=“Description of Artificial Sequence: Synthetic oligonucleotide”
tgcagcctgggaccacttggcatgg

SEQ ID NO: 304 - RNA, 25 nt, Artificial Sequence
  source: /note=“Description of Artificial Sequence: Synthetic oligonucleotide”
ugaggaagcucaucucuccuaugug

SEQ ID NO: 305 - RNA, 25 nt, Artificial Sequence
  source: /note=“Description of Artificial Sequence: Synthetic oligonucleotide”
auuggcccgcuuaacgguccguagg

SEQ ID NO: 306 - RNA, 25 nt, Artificial Sequence
  source: /note=“Description of Artificial Sequence: Synthetic oligonucleotide”
ugaggaucuugagaacagacaugag

SEQ ID NO: 307 - RNA, 25 nt, Artificial Sequence
  source: /note=“Description of Artificial Sequence: Synthetic oligonucleotide”
ugcagaauuccacaaucaccaugag

SEQ ID NO: 308 - DNA, 25 bp, Artificial Sequence
  source: /note=“Description of Combined DNA/RNA Molecule: Synthetic oligonucleotide”
  source: /note=“Description of Artificial Sequence: Synthetic oligonucleotide”
gaguccagaaucctcuuccaugctc

SEQ ID NO: 309 - RNA, 25 nt, Artificial Sequence
  source: /note=“Description of Artificial Sequence: Synthetic oligonucleotide”
gccggcucuuucgcuuacuguucug

SEQ ID NO: 310 - RNA, 25 nt, Artificial Sequence
  source: /note=“Description of Artificial Sequence: Synthetic oligonucleotide”
ugagaugcuccaaggucaggaaguc

SEQ ID NO: 311 - RNA, 25 nt, Artificial Sequence
  source: /note=“Description of Artificial Sequence: Synthetic oligonucleotide”
acagaugagaugcuccaaggucagg

SEQ ID NO: 312 - RNA, 25 nt, Artificial Sequence
  source: /note=“Description of Artificial Sequence: Synthetic oligonucleotide”
augccaagaacuccaugcccuuagc

SEQ ID NO: 313 - RNA, 25 nt, Artificial Sequence
  source: /note=“Description of Artificial Sequence: Synthetic oligonucleotide”
ugugacucuuucaauaaacagcgug

SEQ ID NO: 314 - RNA, 25 nt, Artificial Sequence
  source: /note=“Description of Artificial Sequence: Synthetic oligonucleotide”
ucugugacucuuucaauaaacagcg

SEQ ID NO: 315 - RNA, 25 nt, Artificial Sequence
  source: /note=“Description of Artificial Sequence: Synthetic oligonucleotide”
acaucaccaucagaggcccuccuug

SEQ ID NO: 316 - RNA, 25 nt, Artificial Sequence
  source: /note=“Description of Artificial Sequence: Synthetic oligonucleotide”
uaagucacguuugcucuugagguag

SEQ ID NO: 317 - RNA, 25 nt, Artificial Sequence
  source: /note=“Description of Artificial Sequence: Synthetic oligonucleotide”
aggucccgaugaaugcacuuucugg

SEQ ID NO: 318 - RNA, 25 nt, Artificial Sequence
  source: /note=“Description of Artificial Sequence: Synthetic oligonucleotide”
ucucgcugccaggucccgaugaaug

SEQ ID NO: 319 - RNA, 25 nt, Artificial Sequence
  source: /note=“Description of Artificial Sequence: Synthetic oligonucleotide”
uguuucucgcugccaggucccgaug

SEQ ID NO: 320 - RNA, 25 nt, Artificial Sequence
  source: /note=“Description of Artificial Sequence: Synthetic oligonucleotide”
ugggugccuuuuaaacugaguucag

SEQ ID NO: 321 - RNA, 25 nt, Artificial Sequence
  source: /note=“Description of Artificial Sequence: Synthetic oligonucleotide”
gcugggugccuuuuaaacucaguug